(19) United States
(12) Patent Application Publication
(10) Pub. No.: US 2023/0405185 A1
(43) Pub. Date: Dec. 21, 2023

(54) COMPOSITION FOR PREPARING DECELLULARIZED SCAFFOLD, COMPRISING GRAPHENE NANOSTRUCTURE

(71) Applicants: SEOUL NATIONAL UNIVERSITY R & DB FOUNDATION, Seoul (KR); BIOGO CO., LTD., Seoul (KR)

(72) Inventors: Kyung-Sun KANG, Seoul (KR); Da-Hyun KIM, Gyeonggi-do (KR); Jaechul RYU, Gyeonggi-do (KR)

(21) Appl. No.: 18/254,052

(22) PCT Filed: Nov. 16, 2021
(86) PCT No.: PCT/KR2021/016760

§ 371 Date: May 23, 2023

(30) Foreign Application Priority Data
Nov. 24, 2020 (KR) 10-2020-0158737
Apr. 27, 2021 (KR) 10-2021-0054291

(51) Int. Cl. A61L 27/36; A61L 27/54; B82Y 5/00; A61L 27/08

(52) U.S. Cl. CPC A61L 27/3683; A61L 27/54; B82Y 5/00; A61L 27/08

(57) ABSTRACT

The present invention relates to a composition for preparing a decellularized scaffold, including nano graphene oxide. The present inventors crosslinked nano graphene oxide to a decellularized liver scaffold to strengthen the properties of the scaffold and suppress protease activity of the scaffold, and thus have established an optimum crosslinking condition exhibiting the effects of anti-inflammation and polarization to M2 macrophages. That is, since it is confirmed that nano graphene oxide strengthens the durability of the scaffold so that biodegradation is suppressed and, simultaneously, inflammatory responses that may occur after transplantation are minimized, it is expected that nano graphene oxide can be effectively used in the production and transplantation of clinically applicable artificial organs.

### TECHNICAL FIELD

The present invention relates to a composition for preparing a decellularized scaffold, and the like, including a graphene nanostructure.

The present application claims priority to and the benefit of Korean Patent Application Nos. 10-2020-0158737 and 10-2020-0054291 filed in the Korean Intellectual Property Office on Nov. 24, 2020 and Apr. 27, 2021, respectively, and all the contents disclosed in the specification and drawings of these applications are incorporated in the present application.

### BACKGROUND ART

Although organ transplantation is an epoch-making treatment capable of replacing dysfunctional organs, the number of people on the waiting list for transplantation is rapidly increasing each year due to the serious shortage of donated organs. As part of efforts to replace donor organs, many studies have been conducted on artificial organ production through engineering approaches such as tissue engineering or 3D printing. In particular, decellularized organ scaffolds are known as scaffolds suitable for the production of artificial organs because the decellularized organ scaffolds retain the extracellular components and the fine structure of the organ while removing all the cells from animal organs. Although various artificial organs have been manufactured using this technique, various technical limitations need to be overcome for actual clinical application.

However, it is known that when a decellularized organ scaffold is transplanted in vivo, the decellularized organ scaffold is rapidly degraded by endogenous enzymes, and the degraded products induce an inflammatory response. Therefore, research has been conducted to strengthen the physical properties of biological scaffolds. From this point of view, glutaraldehyde (GA) is a very strong crosslinking agent, but has a disadvantage of being highly cytotoxic and inducing the calcification of the scaffold. Therefore, there is a growing need for developing a crosslinking agent capable of strengthening physical properties and simultaneously improving biocompatibility.

Further, chronic liver diseases such as cirrhosis are diseases that kill about 2 million patients each year. In recent decades, liver transplantation has been conducted in many cases of end-stage liver disease due to breakthrough developments in immunology and organ transplantation, and liver transplantation has been currently recognized as the only treatment for congenital or acquired liver disease, but the number of donors is insufficient. In this respect, the production of an artificial liver through tissue engineering has been attracting attention as a substitute for donor organs. Since Korea has a very high mortality rate due to liver-related diseases compared to other diseases, has a considerable number of people waiting for transplantation due to the shortage of organs for transplantation, and is one of the countries with the highest number of liver transplants, there is a need for developing a technique capable of ameliorating these situations.

Although various methods such as tissue engineering, application of 3D printer technology, and organ reconstruction (organoid) using stem cells have been studied in order to produce a liver scaffold, technical limitations such as the inability to mimic organ fine structures and size limitations have not been overcome. As a human organ-mimicking scaffold with safety and functionality while overcoming the above problems, a decellularized scaffold in which all cells have been removed from animal organs is being evaluated as a useful substrate. Cellular components capable of inducing immune responses are removed, and simultaneously, organ-specific microenvironments such as microstructures and biochemical signals are created in scaffolds, so that there is an advantage in that the three-dimensional organization of cells in the scaffold is facilitated when human cells are injected. Therefore, decellularized scaffolds may be used as suitable substrates for producing artificial organs, and research has been conducted to reconstruct various organs using decellularized scaffolds.

Meanwhile, graphene, which is a sheet of carbon atoms packed in a honeycomb lattice, and its derivatives have been extensively studied in materials science over the last decade due to its special properties such as excellent mechanical stiffness, electrical conductivity, and ease of chemical functionalization. Graphene is one of the carbon allotropes, and has a structure in which carbon atoms are grouped together to form a two-dimensional plane. Each carbon atom forms a hexagonal lattice, and the carbon atoms are located at the vertices of the hexagons. This shape is also called a honeycomb structure or a honeycomb lattice. It is a thin film with a thickness of one atom, and a thickness of 0.2 nm (1 nm is 1/1,000,000,000 m) or 2/10,000,000,000 m, is very thin and has high physical and chemical stability. Among them, graphene nanomaterials have various performances and ripple effects, and there are inexhaustible fields of application as an industry including solutions such as parts and finished products, and related ‘raw material’ equipment using the graphene nanomaterials. However, despite various attempts to strengthen biopolymers, the precise in vivo role of graphene nanostructures as crosslinking agents for organ scaffolds remains unclear.

Therefore, in this technique, nano-sized graphene structures were used as crosslinking agents to suppress post-transplantation biodegradation and inflammatory response, thereby developing bioscaffolds for artificial organs in which biodegradation and transplant rejection are mitigated.

### DISCLOSURE

### Technical Problem

Thus, the present inventors used nano graphene oxide capable of not only strengthening the physical properties of a scaffold that helps cell engraftment during the production of an artificial organ, but also regulating an immune response after transplantation as a crosslinking agent of a decellularized organ scaffold. Nano graphene oxide strengthened physical properties, and not only directly inhibited MMP, which is an extracellular matrix-degrading enzyme, but also promoted polarization to M2 macrophages. Through this, the expression of TIMP, which alleviates inflammation and simultaneously is an MMP inhibitor, was induced to suppress post-transplantation biodegradation and inflammatory response, thereby developing bioscaffolds for artificial organs in which biodegradation and transplant rejection are mitigated.

Therefore, an object of the present invention is to provide a composition for preparing a decellularized scaffold, including a graphene nanostructure.

Another object of the present invention is to provide a composition for strengthening the physical properties of a decellularized scaffold, including a graphene nanostructure.

Still another object of the present invention is to provide a composition for producing an artificial organ, including a graphene nanostructure.

Yet another object of the present invention is to provide a method for producing an artificial organ using a graphene nanostructure.

Yet another object of the present invention is to provide an artificial organ produced by the method.

However, technical problems to be solved by the present invention are not limited to the aforementioned problems, and other problems that are not mentioned may be clearly understood by those skilled in the art from the following description.

### Technical Solution

To achieve the objects of the present invention, the present invention provides a composition for preparing a decellularized scaffold, including a graphene nanostructure.

Further, the present invention provides a composition for strengthening the physical properties of a decellularized scaffold, including a graphene nanostructure.

In addition, the present invention provides a composition for producing an artificial organ, including a graphene nanostructure.

Furthermore, the present invention provides a composition for producing artificial meat, including a graphene nanostructure.

Further, the present invention provides a use of a composition including a graphene nanostructure for preparing a decellularized scaffold.

In addition, the present invention provides a use of a composition including a graphene nanostructure for producing an artificial organ.

Furthermore, the present invention provides a use of a composition including a graphene nanostructure for strengthening the physical properties of a decellularized scaffold.

Further, the present invention provides a method for producing an artificial organ, the method including performing treatment with a composition including a graphene nanostructure.

In addition, the present invention provides a use of a composition including a graphene nanostructure for producing artificial meat.

Furthermore, the present invention provides a method for producing artificial meat, the method including performing treatment with a composition including a graphene nanostructure.

Further, the present invention provides an artificial organ produced by the method.

In addition, the present invention provides artificial meat produced by the method.

In an exemplary embodiment of the present invention, the graphene nanostructure may be a nano-sized graphene oxide or graphene quantum dot, but is not limited thereto.

In another exemplary embodiment of the present invention, the nano-sized graphene oxide may have a thickness of 20 nm or less; or an average diameter of 15 to 50 nm, but is not limited thereto.

In still another exemplary embodiment of the present invention, the graphene nanostructure may be crosslinked to a decellularized scaffold, but is not limited thereto.

In yet another exemplary embodiment of the present invention, the graphene nanostructure may strengthen or improve the physical properties of a decellularized scaffold, but is not limited thereto.

In yet another exemplary embodiment of the present invention, the physical properties may be one or more selected from the group consisting of the following properties, but are not limited thereto:
i) elasticity of the decellularized scaffold;
ii) weight loss due to degrading enzymes of the decellularized scaffold; and
iii) tensile strength of the decellularized scaffold.

In yet another exemplary embodiment of the present invention, the graphene nanostructure may suppress the in vivo degradation of a decellularized scaffold by directly suppressing a matrix metalloproteinase (MMP), but is not limited thereto.

In yet another exemplary embodiment of the present invention, the graphene nanostructure may ameliorate or suppress the induction of inflammatory response of a decellularized scaffold, but is not limited thereto.

In yet another exemplary embodiment of the present invention, the graphene nanostructure may alleviate post-transplantation inflammatory responses by promoting polarization to M2 macrophages, but is not limited thereto. In the present invention, the polarization to M2 macrophages may be polarization to M2c, but is not limited thereto.

In yet another exemplary embodiment of the present invention, the artificial organ may be biocompatible.

In yet another exemplary embodiment of the present invention, the artificial organ is not limited as long as it is an organ including blood vessels, and specifically, may be the liver, but is not limited thereto.

In yet another exemplary embodiment of the present invention, the composition may further include parenchymal cells, but is not limited thereto.

In yet another exemplary embodiment of the present invention, the composition may further include stem cell-derived parenchymal cells, but is not limited thereto.

In yet another exemplary embodiment of the present invention, the composition may further include non-parenchymal cells, but is not limited thereto.

In yet another exemplary embodiment of the present invention, the stem cells may be one or more selected from the group consisting of induced pluripotent stem cells (iPSCs), embryonic stem cells, marrow-derived stem cells, adipose tissue-derived stem cells and placenta-derived stem cells, but are not limited thereto.

In yet another exemplary embodiment of the present invention, the preparation method may include the following steps, but is not limited thereto.
a) providing an organ from a tissue source;
b) decellularizing the provided organ; and
c) treating the decellularized organ with a graphene nanostructure.

In yet another exemplary embodiment of the present invention, the preparation method may further include one or more steps selected from the group consisting of the following steps, but is not limited thereto.
d-1) recellularizing a decellularized organ into vascular endothelial cells;
d-2) recellularizing a decellularized organ into parenchymal cells; and
d-3) recellularizing a decellularized organ into non-parenchymal cells.

Furthermore, the present invention provides a method for strengthening the physical properties of a decellularized scaffold, the method including treating with a graphene nanostructure.

In an exemplary embodiment of the present invention, the method for strengthening the physical properties may include the following steps, but is not limited thereto:
a) providing an organ from a tissue source;
b) decellularizing the provided organ; and
c) treating the decellularized organ with a graphene nanostructure.

### Advantageous Effects

The present inventors have revealed that when nano graphene oxide is used as a crosslinking agent for decellularized scaffolds, not only the physical properties of decellularized scaffolds are strengthened, but also the inflammatory response induced within the scaffold or systemically after transplantation is alleviated. Further, the present inventors confirmed a new mechanism that nano graphene oxide not only promotes anti-inflammatory responses by promoting polarization to M2c macrophages, but also suppresses the biodegradation of the scaffold by suppressing MMP secretion, which is useful in that nano graphene oxide not only increases the engraftment rate of transplants during the production of an artificial organ, but also allows the decellularized scaffold to be maintained in vivo for a long period of time.

Therefore, it is possible to produce an artificial organ in which various cells are applied to a transplantable scaffold prepared using nano graphene oxide, which is expected to contribute to the improvement in the quality of life and the extension of the lifespan of patients at home and abroad as an alternative treatment for organ transplantation. In addition, nano graphene oxide is expected to be widely used in terms of tissue engineering, such as application to not only a decellularized organ scaffold, but also a synthetic biomaterial.

### DESCRIPTION OF DRAWINGS

FIG. 1A illustrates a transmission electron electrical inspection image of the graphite layer structure of an NGO sheet and NGO.

FIG. 1B illustrates the size distribution of NGO according to TEM results.

FIG. 1C illustrates the AFM image of NGO.

FIG. 1D illustrates the height distribution of NGO (n=26) according to AFM results.

FIG. 1E illustrates the Raman spectrum of NGO showing D and G bands at 1350 cm⁻¹ and 1580 cm⁻¹.

FIG. 1F suggests peaks for C—C bonds (51.4%), C—O bonds (38.76%) and C═O bonds (9.85%) according to the high-resolution XPS analysis results of NGO.

FIG. 1G illustrates the FTIR transmission spectrum of NGO showing peaks assigned to hydroxyl, carboxyl and epoxy groups.

FIG. 1H illustrates the zeta potential of NGO showing moderate dispersion stability.

FIG. 2A illustrates a gross image of native liver and dECM liver scaffolds.

FIG. 2B illustrates the histological examination results of native liver (top) and dECM liver (bottom) by H&E staining. Scale bar, 200 μm.

FIG. 2C illustrates the histological examination results of native liver (top) and dECM liver (bottom) by Picrosirius red staining. Scale bar, 200 μm.

FIG. 2D illustrates the SEM analysis results of native liver (top) and dECM liver (bottom).

FIG. 2E illustrates the DNA quantification results of native liver and dECM liver. Each group n=5, mean±SD, ***p<0.001 vs native liver.

FIG. 2F illustrates the results of analyzing the KEGG and Reactome pathways of the top 100 proteins in native liver.

FIG. 2G illustrates the results of analyzing the KEGG and Reactome pathways of the top 100 proteins in ECM liver.

FIG. 2H lists the proteins classified as ECM among the top 100 proteins in dECM liver, and illustrates the results of quantifying the molecular fraction of each protein in dECM liver.

FIG. 2I illustrates the results of analyzing the interaction of ECM proteins in dECM liver.

FIG. 3A is a full image of crosslinked scaffolds, and illustrates the images of PBS (CTL, control), 0.625% GA, 1 μg/mL NGO (NGO-1), 5 μg/mL NGO (NGO-5) and 10 μg/mL NGO (NGO-10) groups.

FIG. 3B illustrates SEM analysis results showing the microstructures of the crosslinked scaffolds. NGOs mixed in ECM fibers are indicated by yellow arrows. Scale bar, 1 μm.

FIG. 3C illustrates the Raman spectroscopy analysis results of the scaffolds crosslinked to CTL scaffold, NGO-1, NGO-5 and NGO-10. The NGO peak appeared at 1580 cm⁻¹ and the dECM scaffold peak appeared at 2930 cm⁻¹.

FIG. 3D illustrates a CTL scaffold crosslinked to NGO-5 and the FTIR transmission spectrum of the scaffold.

FIG. 3E illustrates representative stress-strain curves of scaffolds in each group.

FIG. 3F illustrates the Young's modulus of each scaffold at 5% strain rate. Each group n=4, mean±SD, *p<0.05, **p<0.01, and n.s. shows not statistically significant compared to CTL.

FIG. 3G illustrates the amount of maximum stress which each scaffold can withstand. Each group n=7, mean±SD, *p<0.05, ***p<0.001, and n.s. shows not statistically significant compared to CTL.

FIG. 4A illustrates the Raman spectrum results of the scaffolds in the CTL and GA groups.

FIG. 4B illustrates the FT-IR spectra of scaffolds crosslinked to NGO at different concentrations, showing similar patterns regardless of NGO concentration.

FIG. 4C illustrates the swelling ratio of the crosslinked scaffolds in each group. Each group n=3, mean±SD.

FIG. 5A illustrates the SEM images of the crosslinked scaffolds after treatment with MMP-1 for 2 hours. Scale bar, 1 μm.

FIG. 5B illustrates the results of performing Picrosirius red staining to analyze the histological structures of degraded scaffolds after treatment with MMP-1. Scale bar, 200 μm.

FIG. 5C illustrates the weight loss of scaffolds of each group after treatment with MMP-1. Each group n=5, mean±SD, *p<0.05, **p<0.01, and n.s. not statistically significant compared to CTL.

FIG. 5D illustrates the results of performing a ninhydrin assay to quantify the degree of crosslinking.

FIG. 5E illustrates the results of quantifying insoluble collagen in the scaffolds of the CTL, GA, NGO-1, NGO-5 and NGO-10 groups after treatment with MMP-1.

FIG. 6A illustrates the ¹H NMR spectrum results of NGO only, MMP-1 only and MMP cultured with NGO (MMP-1+NGO).

FIG. 6B illustrates the ¹H NMR spectrum results of NGO only, MMP-9 only and MMP cultured with NGO (MMP-9+NGO).

FIG. 6C illustrates the STD-NMR spectra (top, red) of MMP-1, MMP-2, and MMP-9 in the presence of NGO and each reference spectrum (bottom, black).

FIG. 6D illustrates the results of measuring MMP-1 activity in the presence of 1 μg/mL, 5 μg/mL and 10 μg/mL NGO, vehicle (distilled water) and 1,10-phenanthroline (MMP-1 inhibitor).

FIG. 6E illustrates the results of measuring MMP-2 or MMP-9 activity in the presence of 1 μg/mL, 5 μg/mL and 10 μg/mL NGO, vehicle (distilled water) and doxycycline (non-selective MMP inhibitor).

FIG. 6F illustrates the results of evaluating the zinc chelate activity of 1 μg/mL, 5 μg/mL and 10 μg/mL NGO, the left side is an image of NGO cultured with Zn²⁺, and the right side illustrates the results of measuring absorbance. 10 μM EDTA, which is a potent zinc chelator, was used as a positive control. The absorbance of Zn2+ concentrations cultured with dithizone was normalized to the measured value of DMSO (control).

FIG. 6G illustrates the ¹H NMR spectrum results of NGO only, glutamate only and glutamate cultured with NGO (glutamate+NGO).

FIG. 6H illustrates the ¹H NMR spectrum results of NGO only, histidine only and histidine cultured with NGO (histidine+NGO).

FIG. 7A illustrates the H&E staining results on day 15 of culture of the crosslinked scaffolds which were aliquoted into HepG2 cells in each group.

FIG. 7B illustrates the H&E staining results on day 15 of culture of the crosslinked scaffolds which were aliquoted into HUVEC cells in each group.

FIG. 7C illustrates the results of evaluating the viability of HepG2 cells on the scaffold crosslinked in each group by MTT assay.

FIG. 7D illustrates the results of evaluating the viability of HUVEC cells on the scaffold crosslinked in each group by MTT assay.

FIG. 7E illustrates the results of quantifying the amount of human albumin secreted from each scaffold inoculated with HepG2 cells on days 3, 6, 9 and 12 by ELISA.

FIG. 7F illustrates the results of quantifying the amount of urea secreted from each scaffold inoculated with HepG2 cells on days 3, 6, 9 and 12 by ELISA.

FIG. 7G illustrates the results of quantifying the amount of VEGF secreted from each scaffold inoculated with HUVEC cells on days 3, 6, 9 and 12.

FIG. 7H illustrates the results of quantifying the amount of nitric oxide (NO) secreted from each scaffold inoculated with HUVEC cells on days 3, 6, 9 and 12.

FIG. 8A illustrates the results of quantifying macrophages such as M1 polarized within the crosslinked scaffolds using flow cytometry.

FIG. 8B illustrates the results of quantifying macrophages such as M2 polarized within the crosslinked scaffolds using flow cytometry.

FIG. 8C illustrates representative confocal images of M1 macrophages (C—C chemokine receptor 7; CCR7; M1-specific marker; green, CD68; pan-macrophage marker; red).

FIG. 8D illustrates representative confocal images of M2 macrophages (CD206; M2-specific marker; green, CD68).

FIG. 8E illustrates the ratio of M1 (CD68+CCR7+) and M2 (CD68+CD206+) to the CD68+ population in the CTL, NGO-1, NGO-5 and NGO-10 groups.

FIG. 9A illustrates representative images obtained on day 7 of transplantation of each scaffold.

FIG. 9B illustrates the results of measuring the cross-sectional areas of scaffold remnants obtained 7 days after transplantation.

FIG. 9C illustrates representative images obtained on day 21 of transplantation of each scaffold.

FIG. 9D illustrates the results of measuring the cross-sectional areas of scaffold remnants obtained 21 days after transplantation.

FIG. 9E illustrates the H&E staining results of scaffolds obtained on 7 and 21 days after transplantation (I: transplanted scaffold, S: surrounding tissue). Scale bar, 200 μm.

FIG. 9F illustrates the quantification results of inflammatory cells including neutrophils and lymphocytes, in each scaffold on day 7 after transplantation.

FIG. 9G illustrates the quantification results of inflammatory cells including neutrophils and lymphocytes, in each scaffold on day 21 after transplantation.

FIG. 9H illustrates representative immunohistochemical images of each scaffold on day 7 of transplantation. Scaffolds were each probed with M1 (F4/80; green, CCR7; red) or M2 (F4/80; green, CD206; red) marker, and nuclei were counterstained with DAPI (blue). Scale bar, 100 μm.

FIG. 9I illustrates the ratio of M2+ cells to M1+ cells infiltrated into the transplanted scaffolds on day 7 of transplantation.

FIG. 9J illustrates representative immunohistochemical images of each scaffold on day 21 of transplantation. Scaffolds were each probed with M1 (F4/80; green, CCR7; red) or M2 (F4/80; green, CD206; red) marker, and nuclei were counterstained with DAPI (blue). Scale bar, 100 μm.

FIG. 9K illustrates the ratio of M2+ cells to M1+ cells infiltrated into the transplanted scaffolds on day 21 of transplantation.

FIG. 10A illustrates representative images obtained on day 35 of transplantation of each scaffold.

FIG. 10B illustrates the results of measuring the cross-sectional areas of scaffold remnants obtained 35 days after transplantation.

FIG. 10C illustrates the H&E staining results of scaffolds obtained 35 days after transplantation (I: transplanted scaffold, S: surrounding tissue). Scale bar, 200 μm.

FIG. 10D illustrates the results of quantifying infiltrative neutrophils and lymphocytes within the transplanted scaffolds.

FIG. 10E illustrates representative confocal images of scaffolds 35 days after transplantation in each group stained with DAPI (blue), MMP-9 (green), F4/80 (red) and CCR7 (white). Scale bar, 200 μm.

FIG. 10F illustrates representative confocal images of scaffolds 35 days after transplantation in each group stained with DAPI (blue), TIMP-1 (green), F4/80 (red) and CD206 (white). Scale bar, 200 μm.

FIG. 10G illustrates the ratio of M2+ cells to M1+ cells infiltrated into the transplanted scaffolds.

FIG. 10H illustrates the mean fluorescence intensity of MMP-9 and TIMP-1 in the stained sections of each group.

FIG. 11A illustrates representative images obtained on day 60 of transplantation of each scaffold.

FIG. 11B illustrates the results of measuring the cross-sectional areas of scaffold remnants obtained 60 days after transplantation.

FIG. 11C illustrates inflammatory response in the scaffold through H&E staining of the scaffold remnants of each group.

FIG. 11D illustrates representative confocal images of GA, NGO-5 and NGO-10 group scaffolds stained with M1 and M2 markers.

FIG. 11E illustrates the ratio of M2+ cells to M1+ cells infiltrated into transplanted scaffolds.

FIG. 11F illustrates a schematic view of the mechanism for the pivotal role of NGO in scaffold transplantation.

### MODES OF THE INVENTION

As a result of synthesizing nano-sized graphene oxide (NGO), the present inventors observed sheets having a hexagonal carbon structure, and observed NGOs having a diameter of 30 nm and a thickness of 3 to 8 nm (see Example 1).

Furthermore, as a result of preparing a decellularized liver scaffold, it was confirmed that proteins related to ECM tissue were highly expressed rather than liver metabolism as decellularization proceeds (see Example 2).

Further, a dECM liver scaffold to which NGO was crosslinked was prepared, and it was confirmed that NGO improves the physical properties of the prepared scaffold. Specifically, it was confirmed that mechanical properties such as elastic modulus and maximum tensile strength were strengthened by NGO (see Example 3).

In addition, since it was confirmed that the microstructure of the NGO-crosslinked scaffold was not destroyed despite treatment with MMP-1, which is known as a collagenase, and the weight loss was also remarkably reduced compared to the control, it was confirmed that the crosslinking of NGO strengthens resistance to enzymatic degradation (see Example 4).

Furthermore, it was confirmed that NGO significantly reduced the catalytic activity of MMP-1, MMP-2 and MMP-9, which are involved in cleaving the main components of the scaffold, NGO directly suppresses MMP enzyme activity by directly binding to the scaffold, and NGO suppresses MMP enzyme activity by directly binding glutamate and histidine, which are components in a zinc-binding motif (see Example 5).

Further, as a result of treating hepatic parenchymal cells and vascular endothelial cells with the NGO crosslinked scaffold, it was confirmed that in the NGO-crosslinked scaffold, the cells showed high viability and included a proliferative cell population compared to the control. In addition, it was confirmed that liver functions including albumin secretion, urea secretion and glycogen synthesis in the NGO crosslinked group were significantly increased compared to comparative groups (see Example 6).

Furthermore, it was confirmed that as the NGO-crosslinked scaffold may provide an optimized environment for human M2 polarization, the degradation of the transplanted scaffold and the inflammation induced by transplantation may be ameliorated (see Example 7).

Further, as a result of transplanting the NGO-crosslinked scaffold in vivo in mice, it was confirmed that the area and mass of the NGO-crosslinked scaffold were maintained well without degradation even after a considerable period of time after transplantation, and inflammatory cells such as neutrophils and lymphocytes were considerably reduced. In addition, it was confirmed that the NGO-crosslinked scaffold had a remarkably better M2 polarization effect than the comparative groups (see Example 8).

Furthermore, since it was confirmed that the NGO-crosslinked scaffold released both MMP and TIMP, the levels of M2c macrophages which structurally regulate tissue remodeling were significantly high, it was confirmed that NGO interacted with the released MMP and suppressed the activity of MMP (see Example 9).

Further, it was confirmed that the NGO-crosslinked scaffold exhibited a low level of inflammatory response even after 60 days post-implantation, and the degree of degradation was also lower than those of other comparative groups (see Example

Therefore, the present inventor provides a composition for preparing a decellularized scaffold, including a graphene nanostructure, and a composition for producing an artificial organ.

Hereinafter, the present invention will be described in detail.

The present invention relates to a composition for preparing a decellularized scaffold, including a graphene nanostructure, and a composition for producing an artificial organ.

As used herein, the term “graphene” means that a plurality of carbon atoms are covalently linked to each other to form a polycyclic aromatic molecule, and the covalently linked carbon atoms form a 6-membered ring as a basic repeating unit, but it is also possible to further include a 5-membered ring and/or a 7-membered ring.

As used herein, the term “graphene nanostructure” refers to a nano-sized graphene derivative, and includes nano-sized graphene oxide (nan-GO; NGO) and/or graphene quantum dot (GQD).

As used herein, the term “graphene oxide” is also called graphene oxides and may be abbreviated as “GOs”. Graphene oxide may include a structure in which a functional group containing an oxygen atom such as a carboxyl group, a hydroxy group, or an epoxy group is bonded on graphene, but is not limited thereto.

As used herein, the term “nano-sized graphene oxide (nano-GO)” refers to graphene oxide produced in the form of particles having a nanometer-level size, and may include a structure in which a functional group containing an oxygen atom such as a carboxyl group, a hydroxy group, or an epoxy group is bonded on graphene, but is not limited thereto. The nano-sized graphene oxide may refer to plate-like particles having a predetermined thickness of 20 nm or less and an average diameter of about 15 to 50 nm. For example, the nano-sized graphene oxide may be commercially purchased and used, or may be prepared by methods known in the art, but is not limited thereto. For example, the nano-sized graphene oxide may have an average diameter of 15 to 45 nm, 18 to 45 nm, 20 to 40 nm, 25 to 35 nm, or 27 to 33 nm. Moreover, the nano-sized graphene oxide may be a particle having a thickness of 1 to 20 nm, 1 to 19 nm, 1 to 18 nm, 1 to 17 nm, 1 to 16 nm, 1 to 15 nm, 1 to 14 nm, 1 to 13 nm, 1 to 12 nm, 1 to 11 nm, 1 to 10 nm, 1 to 9 nm, 2 to 15 nm, 2 to 14 nm, 2 to 13 nm, 2 to 12 nm, 2 to 11 nm, 2 to 10 nm, 2 to 9 nm, or 3 to 8 nm, but is not limited thereto. In an exemplary embodiment of the present invention, nano-sized graphene oxide having an average diameter of about 30 nm and a thickness of 3 to 8 nm was synthesized.

As used herein, the term “graphene quantum dot (GQD)” refers to graphene having nano-sized fragments, the graphene quantum dot may be a graphene particle having width, length and height of several nanometers prepared through appropriate processing, and may be obtained by thermo-oxidative cutting of carbon fibers, but the preparation method is not limited thereto. For example, the graphene quantum dot may be a particle having an average diameter of 1 to 5 nm and a thickness of 0.5 to 3 nm, but is not limited thereto. For example, the graphene quantum dot may have an average diameter of 1 to 3 nm or 3 to 5 nm. Moreover, the graphene quantum dot may have a height of 0.5 to 2.5 nm, 0.5 to 1.5 nm, or 1.5 to 2.5 nm.

In the present invention, the graphene nanostructure may be included in the composition at 0.1 to 100 μg/mL, 0.1 to 90 μg/mL, 0.1 to 80 μg/mL, 0.1 to 70 μg/mL, 0.1 to 60 μg/mL, 0.1 to 50 μg/mL, 0.1 to 40 μg/mL, 0.1 to 30 μg/mL, 0.1 to 20 μg/mL, 0.5 to 20 μg/mL, 0.5 to 15 μg/mL, 1 to 10 μg/mL, 1 to 9 μg/mL, 1 to 8 μg/mL, 1 to 7 μg/mL, 1 to 6 μg/mL, 2 to 6 μg/mL, 3 to 6 μg/mL, 4 to 6μg/mL, or about 5 μg/mL, but is not limited thereto.

Tissue engineering is a method of anchoring cells on a certain construct (template) using a combination of cells and various materials, and such a construct (template) is called a scaffold, and the purpose of making this is to create a construct capable of replacing a damaged tissue or organ, that is, an organ. Although there are various types, a construct (template) that leaves only outlines of the microstructure and the organ after cells are removed by decellularizing a biological construct (that is, organ) is also called a bioscaffold or a decellularized scaffold.

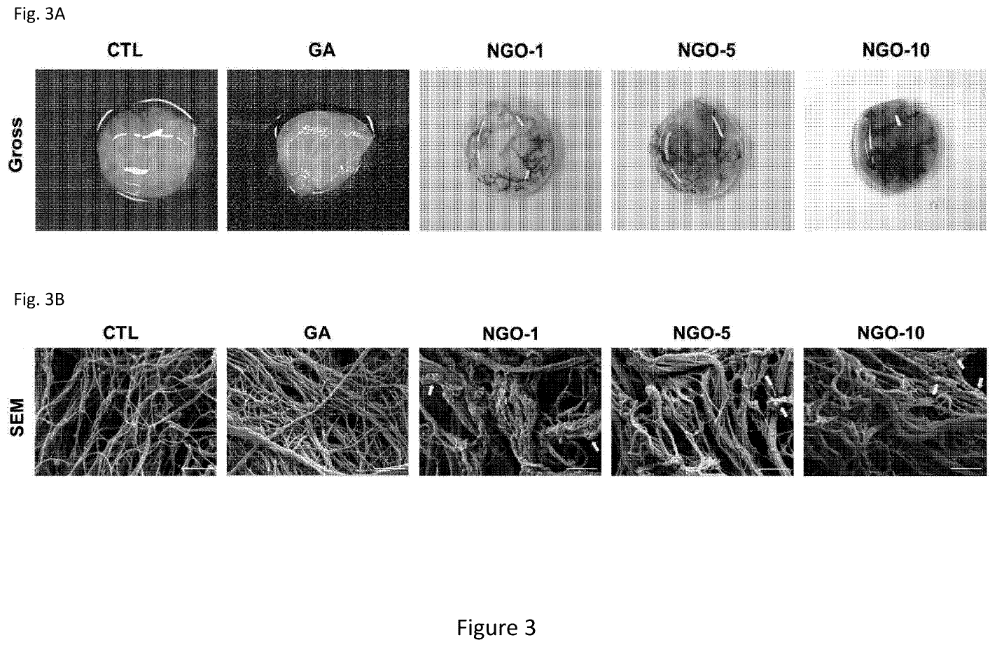

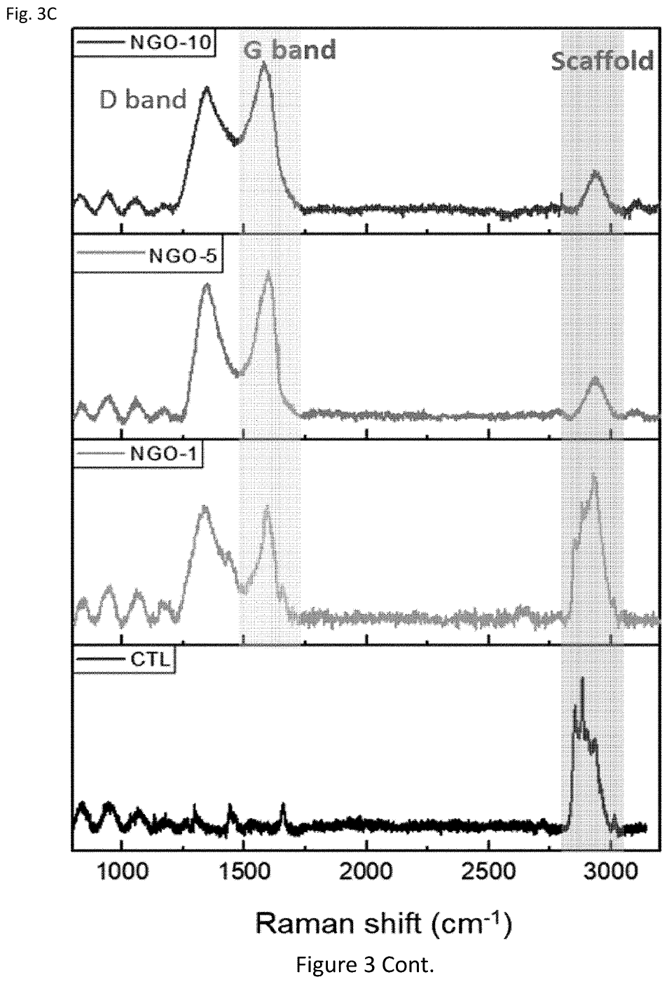

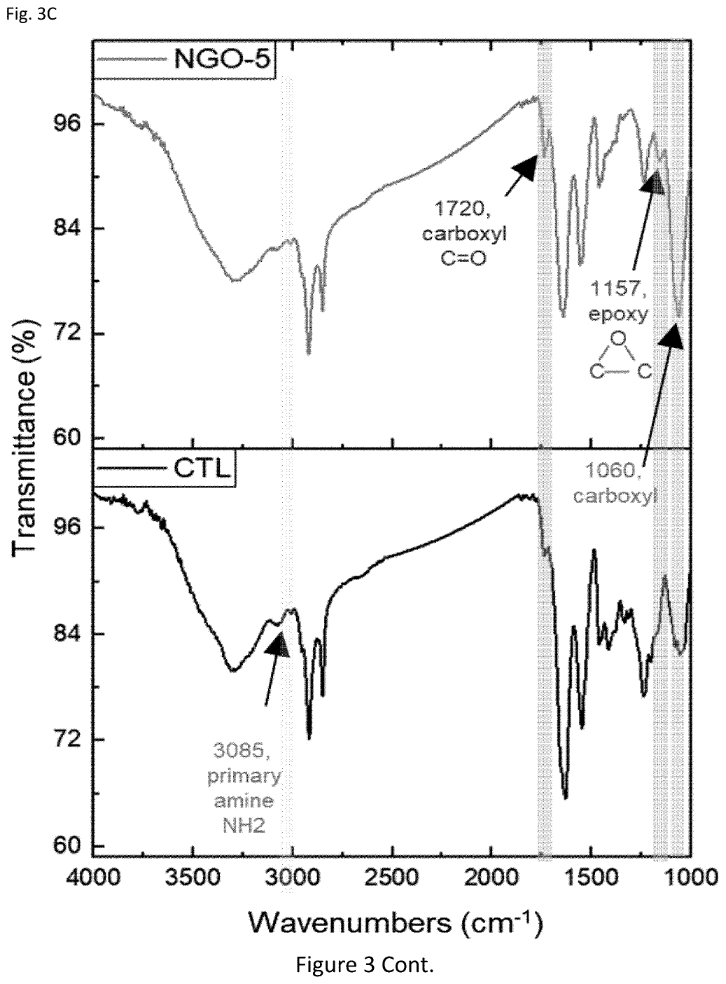

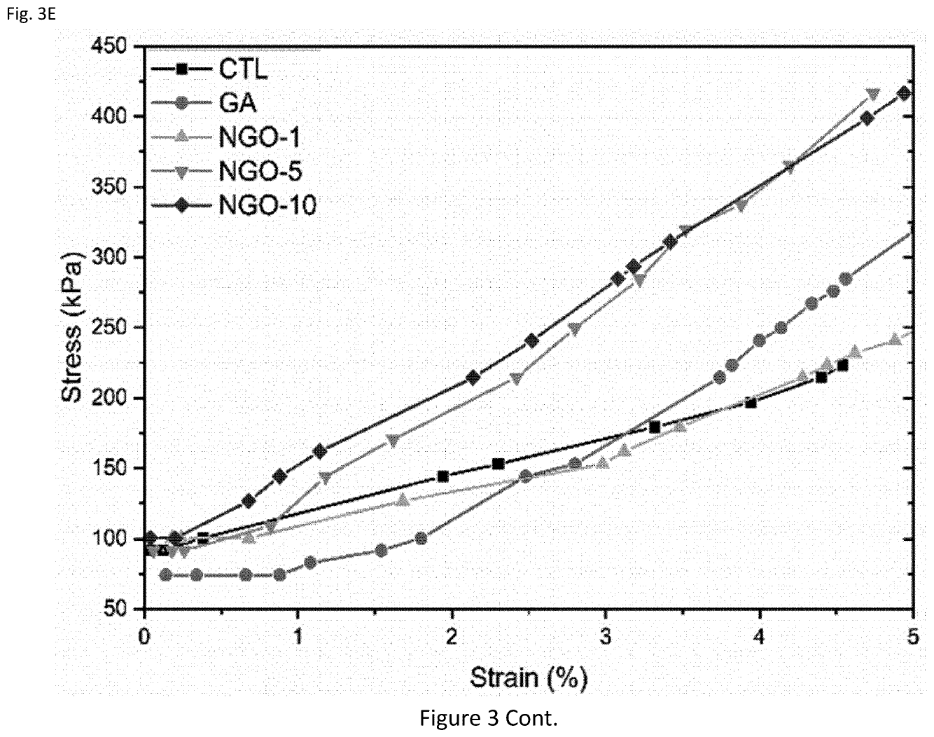

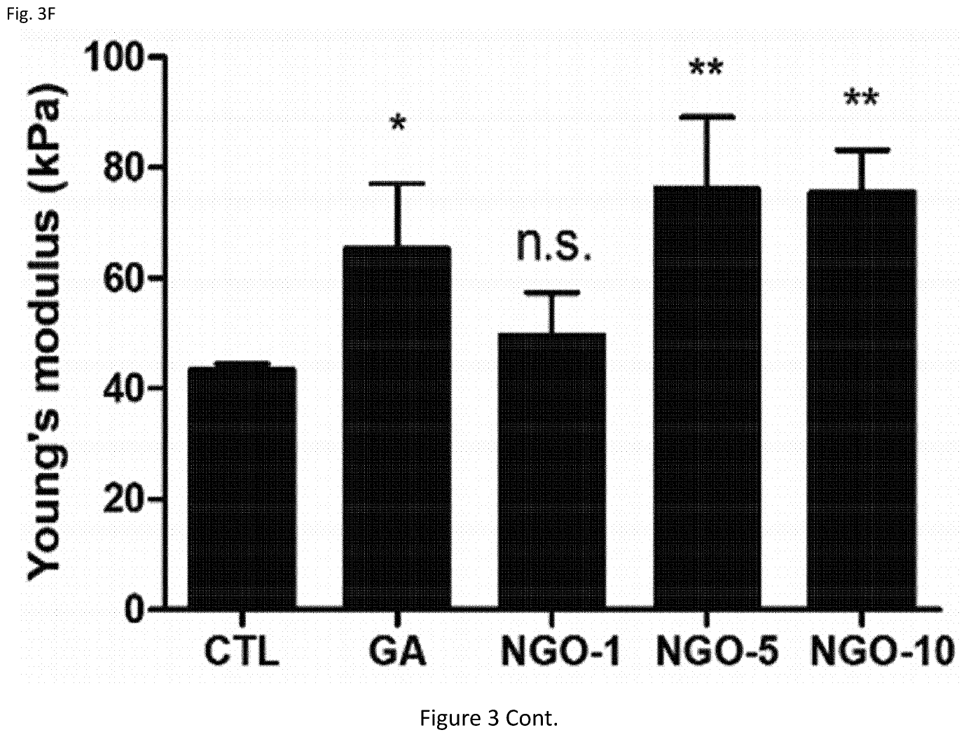

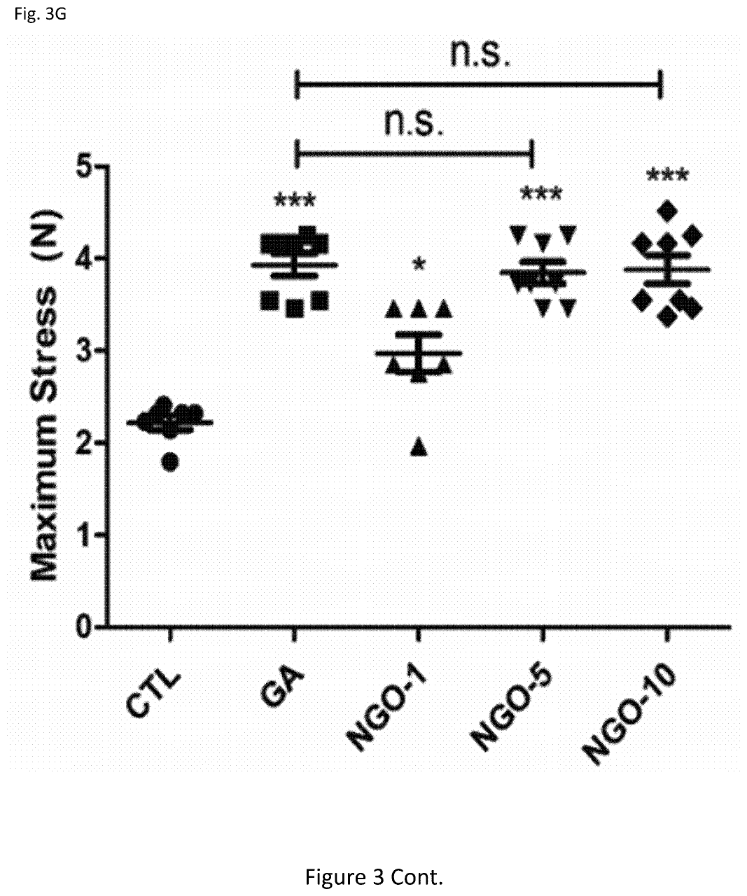

In the present invention, the graphene nanostructure may be crosslinked to the decellularized scaffold, but is not limited thereto. According to an exemplary embodiment of the present invention, the carboxyl group of NGO may be crosslinked to a decellularized scaffold by reacting with an amine group in tissue ECM proteins (collagen, fibronectin, and the like) to form peptide bonds, but is not limited thereto (see FIG. 3D of the present invention).

In the present invention, the graphene nanostructure may induce crosslinking of the tissue in the decellularized scaffold, but is not limited thereto. That is, the composition of the present invention may be a composition for inducing crosslinking of tissue in a decellularized scaffold, but is not limited thereto.

In the present invention, the graphene nanostructure may strengthen or improve the physical properties of a decellularized scaffold, but is not limited thereto. The physical properties are properties that the decellularized scaffold has, such as electrical properties, mechanical properties, thermodynamic properties, magnetic properties, and vibration-related properties.

In the present invention, the physical properties may be one or more selected from the group consisting of the following properties, but are not limited thereto:
i) elasticity of the decellularized scaffold;
ii) weight loss due to degrading enzymes of the decellularized scaffold; and
iii) tensile strength of the decellularized scaffold.

As used herein, the term “elasticity” may be expressed in terms of Young's modulus, and the Young's modulus is a mechanical property that measures the stiffness of a solid material. The elasticity of the present invention may refer to the property of a material deformed by pressure or tension to return to its original shape when the load is removed from the material, but is not limited thereto.

As used herein, the term “tensile strength (TS)” is a force that expresses the strength of a material, and refers to a value obtained by dividing the maximum load that a material can withstand when the material is pulled to be cut by the cross-sectional area of the material. Tensile strength is also called tensile force strength.

In the present invention, the degrading enzyme may refer to an enzyme that catalyzes the breaking of various chemical bonds other than hydrolysis and oxidation in biochemistry, but is not limited thereto. The degrading enzyme may be, for example, lysozyme, an extracellular matrix degrading enzyme (MMP), a collagenase, and the like, but is not limited thereto.

In an exemplary embodiment of the present invention, since it was confirmed that nano-sized graphene oxide, which is a graphene nanostructure, directly suppresses extracellular matrix degrading enzymes (MMP-1, MMP-2, and MMP-9), it was confirmed that the decellularized scaffold is rapidly degraded in vivo (see Example 5 of the present invention).

In the present invention, the graphene nanostructure may ameliorate or suppress the induction of inflammatory response of a decellularized scaffold, but is not limited thereto.

In the present invention, the graphene nanostructure may promote polarization to M2 macrophages, but is not limited thereto.

As used herein, the term “inflammation” is one of the biological responses to harmful stimuli, and refers to a protective response in which immune cells, blood vessels, and inflammatory mediators are involved. The inflammation of the present invention may be a non-specific response mediated by innate immunity, but is not limited thereto.

In the present invention, the innate immune response is a primary defense action that first protects our body from externally invading pathogens, and is also called non-specific immunity. Neutrophils, monocytes, macrophages, and the like are involved in the innate immune response, and the innate immune response occurs rapidly at the time of pathogen invasion and infection, regardless of the type of pathogen or the presence or absence of infection experience. In particular, macrophages are cells which are distributed in all tissues in vivo and are responsible for immunity, and are involved in the elimination of invading pathogens, the elimination of virus-infected autologous cells and cancer cells, and the induction of inflammatory responses. Macrophages may be classified as tissue-resident macrophages differentiated from the yolk sac or fetal liver) during the development process, and monocyte-derived macrophages in which monocytes (differentiated from bone marrow cells) in blood are differentiated by inflammatory responses or invasion of pathogens.

M1 macrophages are induced by IFN-γ, TNF-α and the like, which are cytokines of Th1 cells, and act on the induction of Th1 response, the induction of inflammatory responses, the suppression of cancer growth, and the like. M2 macrophages are induced by IL-4, IL-10 and the like, which are cytokines of Th2 cells, and act on the induction of Th2 response, the suppression of inflammatory responses, the restoration of damaged tissues, and the like. In this manner, bone marrow-derived macrophages and monocyte-derived macrophages may differentiate into M1 or M2 macrophages which act differently according to the type of cytokine. The transition from macrophage M1 type to M2 type is recognized as an essential element in the resolution stage of inflammation. It is known that transitioned M2 macrophages secrete immunosuppressive cytokines (TGF-β and IL-10 and eliminate surrounding dead immune cells by phagocytosis to resolve inflammation and promote tissue repair. It is reported that even in inflammatory diseases, inflammation can be ameliorated and treated by regulating the transition of M1 macrophages to M2 macrophages (M1→M2) to induce the disappearance of inflammation. Therefore, since the graphene nanostructure of the present invention regulates polarization to M2 macrophages, it is expected that a decellularized scaffold and an artificial organ produced therefrom can suppress the in vivo inflammatory response.

In an exemplary embodiment of the present invention, since it was confirmed that nano-sized graphene oxide, which is a graphene nanostructure, promotes polarization to M2c macrophages, which are known to mediate matrix remodeling in addition to immunoregulation among M2 macrophages, it was confirmed through tissue remodeling that the decellularized scaffold can be maintained in vivo for a long period of time (see Example 7 of the present invention).

The artificial organ of the present invention may be characterized in that the decellularized support is treated with the graphene nanostructure to strengthen physical properties and suppress or alleviate inflammatory responses, but is not limited thereto. Specifically, the graphene nanostructure of the present invention may be crosslinked to the tissues and blood vessels, and the like of the decellularized scaffold to strengthen the physical properties of the decellularized scaffold and alleviate or suppress an inflammatory response induced by the decellularized support after transplantation.

Therefore, the artificial organ may be biocompatible. The term “biocompatibility” in the present invention is used interchangeably with “transplant compatibility” and refers to a property of not causing immune rejection upon organ transplantation. A cell, tissue, or organ may be rendered transplant compatible by reducing the expression of genes, proteins or enzymes responsible for immune rejection in an artificial organ, and as a non-limiting example thereof, suppressing the expression of the HLA gene, the activity of alpha-1,3-galactosyltransferase, and the like may render a cell, tissue or organ transplant compatible.

Further, the biocompatibility of the present invention may be obtained by suppressing proteins or enzymes known as immune response inducing epitopes in an organ tissue decellularized scaffold derived from non-primate mammals, and accordingly, it is expected that the graphene nanostructure can be applied to decellularized scaffolds derived from not only wild-type (WT) but also transgenic animals.

In the present invention, the artificial organ is not limited as long as it is an organ including blood vessels, and may be specifically a liver, but is not limited thereto.

In the present invention, the composition may typically be delivered to a tissue or organ matrix by a cell-miscible solution (for example, a physiological composition) under physiological conditions (for example, 37° C.). The composition may include a buffer, a nutrient (for example, a sugar and carbohydrate), an enzyme, a proliferation and/or differentiation medium, a cytokine, an antibody, an inhibitory factor, a growth factor, a salt solution, or a serum-derived protein, and the like, but is not limited thereto.

In the present invention, the composition may further include parenchymal cells, but is not limited thereto. As used herein, the term “parenchymal cells” refers to cells that form the main part that performs functions inherent to cells. In the present invention, the parenchymal cells may specifically be hepatic parenchymal cells, renal parenchymal cells, corneal parenchymal cells, vascular endothelial cells, and the like, preferably hepatic parenchymal cells, that is, hepatocytes, but are not limited thereto. In the present invention, hepatic parenchymal (parenchymal hepatocyte) cells make up a total of 80% of the liver under non-pathological conditions.

In the present invention, the parenchymal cells may be derived from the same species as a recipient, but are not limited thereto.

In the present invention, the composition may further include stem cell-derived parenchymal cells, but is not limited thereto. The stem cell-derived parenchymal cells may be parenchymal cells differentiated from human universal induced pluripotent stem cells from which human leukocyte antigens have been removed and/or which overexpress “Don't eat me” signals, but are not limited thereto.

In the present invention, the composition may further include non-parenchymal cells, but is not limited thereto. The non-parenchymal cells may be one or more selected from the group consisting of hepatic endothelial cells, Kupffer cells, hepatic stellate cells, Ito cells, lipocytes, fat-storing cells, cholangiocytes, pit cells, vascular epithelial cells and fibroblasts, but are not limited thereto.

As used herein, the term “stem cells” refers to cells which have the property capable of continuously producing the same cells as themselves for a certain period of time in an undifferentiated state and the property of differentiating into specific cells under suitable conditions.

In the present invention, the stem cells may be one or more selected from the group consisting of induced pluripotent stem cells (iPSCs), embryonic stem cells, marrow-derived stem cells, adipose tissue-derived stem cells and placenta-derived stem cells, but are not limited thereto.

In addition, the present invention provides a method for producing an artificial organ, the method including treating with a graphene nanostructure.

In the present invention, the method for producing artificial organs may include the following steps, but is not limited thereto:
a) providing an organ from a tissue source;
b) decellularizing the provided organ; and
c) treating the decellularized organ with a graphene nanostructure.

In the present invention, the method may further include one or more steps selected from the group consisting of d-1) recellularizing the decellularized organ into vascular endothelial cells;
d-2) recellularizing the decellularized organ into parenchymal cells; and
d-3) recellularizing the decellularized organ into non-parenchymal cells, but is not limited thereto.

In the present invention, the tissue source may be selected from various mammalian groups such as humans, monkeys, mice, rats, pigs, cows and rabbits.

In the present invention, the decellularization may be performed by methods known in the art, but in an exemplary embodiment of the present invention, the hepatic portal vein obtained from the tissue source is washed with deionized water and PBS, then washed with deoxyribonuclease, and then sterilized with peroxyacetic acid. For example, the following references describe the perfusion-based decellularization of the lungs, liver, kidneys, brain and limbs: Van Putte et al., 2002, Ann. Thorac. Surg., 74(3):893-8; den Butter et al., 1995, Tramp'. Int., 8:466-71; Firth et al., 1989, Clin. Sci. (Lond.), 77(6):657-61; Mazzetti et al., 2004, Brain Res., 999(1):81-90; Wagner et al., 2003, J. Artif. Organs, 6(3):183-91. As an alternative to perfusion-based decellularization, biological tissues and organs may be decellularized by immersion in a decellularization solution that removes cells. See, for example, US Pat. Nos. 6,376,244 and 6,753,181.

In the present invention, physiological buffers suitable for perfusion include nutrient supplies that may be used for storage and/or organ perfusion, including transplantation, and examples thereof include buffers with glucose, EGM-2, EGM-2MV, DMEM, Promocell endothelial cell medium, Medium 200, and DMEMF/12, but include culture medium solution or phosphate buffered saline (PBS) suitable for endothelial cell culture, which is not limited thereto, but are not limited thereto.

In the present invention, alternating the direction of perfusion (for example, anterograde and retrograde) during decellularization may help effectively remove cells from the whole organ or tissue. The decellularization as described in the present specification may cause little damage to the ECM by essentially removing cells from within the organ, but is not limited thereto. Organs or tissues may be decellularized at a suitable temperature between 4 to 40° C. Depending on the size and weight of the organ or tissue, the specific detergent(s) in a cell disintegration medium, and the concentration of the detergent(s), the organ or tissue is generally perfused with the cell disintegration medium for about 2 to about 12 hours per gram of solid organ or tissue. Organs including lavage fluid may be perfused for about 1 to about 12 hours per gram of tissue. Perfusion is generally regulated by physiological conditions including pulsatile blood flow, flow velocity and pressure.

In the present invention, for recellularization to generate an organ or tissue, the number of regenerative cells introduced into and onto a decellularized organ may vary depending on both the type and developmental stage of an organ (for example, what the organ is and the size and weight of the organ) or tissue and regenerative cell. Different types of cells may have different tendencies with respect to the population density that these cells reach. Similarly, different organs or tissues may be recellularized at different densities. For example, a decellularized organ or tissue may be “inoculated” with at least 1,000, 10,000, 100,000, 1,000,000, 10,000,000 or 100,000,000 regenerative cells; or may have about 1,000 cells/tissue mg (wet weight, that is, weight before decellularization) to about 100,000,000 cells/tissue mg (wet weight) after recellularization, but is not limited thereto.

In the present invention, the method may further include culturing an organ treated with a graphene nanostructure in a bioreactor for 1 to 100 days, 1 to 90 days, 1 day to 80 days, 1 day to 70 days, 1 day to 60 days, 1 day to 50 days, 1 day to 40 days, 1 day to 1 month, 1 day to 3 weeks, 1 day to 15 days, 1 day to 2 weeks, 5 days to 15 days, 5 days to 2 weeks, 1 week to 3 weeks or about 2 weeks, but is not limited thereto.

In addition, the present invention provides an artificial organ produced by the method.

The artificial organ may not induce immune rejection in a recipient after transplantation as an organ for transplantation, but is not limited thereto.

The artificial organ may be used for therapeutic agent screening applications, preferably for hepatic disease therapeutic agent screening applications, but is not limited thereto.

Furthermore, the present invention relates to a composition for producing artificial meat, including a graphene nanostructure.

In the present invention, artificial meat includes meat substitute or cultured meat, but is not limited thereto.

In the present invention, the artificial meat may be used to produce fish or meat and processed foods thereof (for example, patties, dumpling fillings, sausages, bulgogi, jerky, and the like), but is not limited thereto.

As used herein, cultured meat refers to edible meat obtained by collecting animal cells and proliferating the animal cells using cell engineering technology. Without manipulating genes, the stem cells of normal livestock are extracted from fetuses by slaughtering pregnant cows and grown in a culture solution to produce red meat. In addition, since muscle cells and muscle fibers are slightly different, a variety of additional processes are required to artificially culture muscles in addition to culture. Cultured meat has the best production efficiency relative to energy among existing meat production methods, and has an advantage of being able to consume meat without killing life.

In the present invention, the process of producing cultured meat may include collecting tissue from a living animal and then isolating stem cells from the tissue, but is not limited thereto. Thereafter, the isolated stem cells may be cultured as muscle cells in a laboratory, grown for several weeks, and then subjected to coloration of muscle fibers, mixing with fat, and the like to produce cultured meat, but are not limited thereto. In this case, in the production process, scaffolds may be used, or a self-assembly method may also be used. Cultured meat can replace saturated fatty acids, which are harmful to the human body, with beneficial fatty acids such as omega-3 during the production process, and may be produced by regulating the medium and culture conditions to select meat that is beneficial to health. In the present invention, the production process of cultured meat may include a known method in the related art or a production process of cultured meat to be developed later, but is not limited thereto.

In the present invention, the cultured meat may be cultured or produced from non-human animal-derived cells, but is not limited thereto. The non-human animal-derived cells are pluripotent stem cells (PSCs) and/or cells differentiated therefrom. The PSCs are induced PSCs (iPSCs) reprogrammed from somatic non-human animal cells and/or cells differentiated therefrom, and the PSCs may be non-embryonic stem cells or embryonic stem cells, but are not limited thereto.

In the present invention, the non-human animal-derived cells may be pluripotent stem cells differentiated into muscle cells, pluripotent stem cells differentiated into fat cells (adipocytes) and/or progenitors thereof, pluripotent stem cells differentiated into interstitial cells (connective tissue) and/or progenitor cells thereof, or pluripotent stem cells differentiated into endothelial cells (blood vessel) and/or progenitor cells thereof, but are not limited thereto.

In the present invention, the non-human animal-derived cells may be satellite cells differentiated into muscle cells and/or progenitor cells thereof, but are not limited thereto.

In the present invention, the non-human animal-derived cells may be one or more selected from the group consisting of muscle cells, adipocytes, interstitial cells, fibroblasts, perivascular cells, endothelial cells and progenitor cells thereof, but are not limited thereto.

In the present invention, the non-human animals may be selected from the group consisting of cattle, sheep, pigs, poultry, crustaceans and fish, but are not limited thereto.

In the present invention, meat substitute is a non-animal food material produced similarly to meat in terms of shape and texture. Most meat substitutes are made from plant-based materials such as soy protein or wheat gluten, and thus are also called plant-based meats, and there are also non-vegetable meat substitutes that utilize my coprotein.

In order to produce high-quality artificial meat, it is important to make the artificial meat in a form similar to real meat not only in taste but also in textural properties.

In an exemplary embodiment of the present invention, a dECM liver scaffold to which NGO was crosslinked was prepared, and it was confirmed that NGO improves the physical properties of the prepared scaffold. Specifically, it was confirmed that mechanical properties such as elastic modulus and maximum tensile strength were strengthened by NGO (see Example 3). In addition, since it was confirmed that the microstructure of the NGO-crosslinked scaffold was not destroyed despite treatment with MMP-1, which is known as a collagenase, and the weight loss was also remarkably reduced compared to the control, it was confirmed that the crosslinking of NGO strengthens resistance of the scaffold to enzymatic degradation (see Example 4).

Therefore, it is expected that by strengthening the physical properties of artificial meat by treatment with NGO at the culturing stage of artificial meat, it is possible to produce artificial meat capable of satisfying sensory preferences such as textural properties and texture.

Throughout the specification of the present application, when one part “includes” one constituent element, unless otherwise specifically described, this does not mean that another constituent element is excluded, but means that another constituent element may be further included. Throughout the specification of the present application, a term of a degree, such as “about” or “substantially”, is used in a corresponding numerical value or used as a meaning close to the numerical value when natural manufacturing and material tolerance errors are presented in a described meaning, and is used to prevent an unconscientious infringer from illegally using disclosed contents including a numerical value illustrated as being accurate or absolute in order to help understanding of the present invention. Throughout the specification of the present application, a term of a degree, such as a “step . . . ” or a “step of . . . ”, does not mean a “step for . . . ”.

Throughout the specification of the present application, the term “combination(s) thereof” included in the Markush type expression means a mixture or combination of at least one selected from the group consisting of constituent elements described in the Markush type expression, and means including at least one selected from the group consisting of the constituent elements.

Throughout the specification of the present application, the description “A and/or B” means “A or B, or A and B”.

Specific steps may be performed out of the order described in cases where certain embodiments can be implemented differently. For example, two steps described in succession may be performed substantially concurrently, or may be performed in reverse order to that described.

Hereinafter, preferred examples for helping with understanding of the present invention will be suggested. However, the following examples are provided only so that the present invention may be more easily understood, and the content of the present invention is not limited by the following examples.

### EXAMPLES

### Experimental Methods and Materials

### Preparation and Analysis of Nano Graphene Oxide (NGO)

NGO was easily synthesized from graphite through Taylor-Couette flow. The morphology and size distribution of the synthesized NGO particles were analyzed with a Cs-corrected transmission electron microscope (JEM-ARM200F, Cold FEG, JEOL Ltd, Japan). The NGO was loaded onto 400-mesh carbon-coated copper grids and completely dried before analysis. Next, the NGO prepared on a sapphire wafer was examined with an atomic force microscope (XE-100, Park Systems, Korea) in non-contact mode. An area of 25 μm² was scanned. Raman spectra of the NGO were obtained using a 532 nm excitation laser (LabRAM HR Evolution, HORIBA, Japan) of a Raman spectrometer. NGO powder was confirmed by an X-ray photoelectron spectrometer (AXIS-His, Kratos, USA). An FTIR spectrophotometer (Nicolet 6700, Thermo Fisher Scientific, USA) was performed with the existing KBr pellet method to obtain the FTIR spectra of the NGO. The zeta potential of the NGO was analyzed using a Zetasizer Nano ZSP (Malvern Instruments, England).

### Fabrication and Characterization of Decellularized Liver Scaffold

All rat experiments were performed in accordance with the approved guidelines of the Seoul National University Animal Care Committee (SNU-191208-1). Livers were collected from female Sprague-Dawley rats (200 to 250 g) by catheterization into the portal vein and bile duct in order to fabricate a decellularized rat liver scaffold. Subsequently, livers were decellularized with 0.1% sodium dodecyl sulfate (SDS) through perfusion of the portal vein, washed with PBS and sterilized with 0.1% peracetic acid (Sigma). Samples were subjected to histological examination and scanning electron microscopy. DNA was extracted from the samples according to the manufacturer's instructions using an AccuPrep Genomic DNA extraction kit (Bioneer, Republic of Korea).

DNA concentrations were measured with a spectrophotometer (Nanodrop 2000c, Thermo Fisher Scientific) and normalized to the dry weight of each sample.

Protein body analysis was performed using a liquid chromatography hybrid-FT orbitrap mass spectrometer (Korea Basic Science Institute, Ochang, Republic of Korea) to determine the protein properties of the liver scaffold. The proteomic profiles of native liver and dECM liver were analyzed using MaxQuant. The top 100 proteins were classified, and KEGG and Reactome pathway analysis was performed using STRING (string-db.org). Among the top 100 proteins which appeared in the dECM liver, proteins classified as ECM components were subjected to interactome analysis using STRING.

### Histological Examination

Tissue samples were fixed in 4% formaldehyde at 4° C. overnight and subjected to tissue processing. The dehydrated samples were embedded in paraffin and cut into 10 μm sections. The tissue sections were deparaffinized and hydrated with a series of ethanol washes while decreasing the concentration. Tissue slides were stained with picrosirius red (0.1% Direct red 80, Sigma, 0.1% Fast green FCF, Sigma) and hematoxylin (Sigma) & eosin (Muto Pure Chemicals, Japan) according to the manufacturer's instructions. A PAS staining kit (ab150680, Abcam) was used for periodic acid-Shiff staining (PAS). After staining, the slides were washed with tap water, dehydrated through an ascending series of ethanol ranging from 70% to 100%, and then washed with xylene. The samples were visualized with a Nikon optical microscope and NIS-Elements software (Nikon, Japan).

### Scanning Electron Microscope (SEM)

Sample specimens were fixed with 4% glutaraldehyde and 1% paraformaldehyde in a buffer solution of 0.1 M cacodylate buffer. Next, the samples were rinsed with cacodylated buffer and dehydrated using a series of graded ethanol. After critical point drying, the sputter-coated samples were visualized using a variable pressure FE-SEM (SUPRA55VP, Carl Zeiss, Germany).

### Crosslinking to NGO

After the decellularized liver scaffold was sterilized, 50 mL of each crosslinking agent (PBS; negative control, 0.625% glutaraldehyde; positive control, 1 μg/mL, 5 μg/mL and 10 μg/mL NGO) was perfused through the bile duct and portal vein of the entire liver scaffold for 24 hours. After the crosslinked scaffold was washed with deionized water, a scaffold disc (r=6 mm) was fabricated using a skin biopsy punch (P1250, Acuderm Inc., USA).

The crosslinked scaffold disc was used while being stored in PBS containing antibiotics at 4° C. The lyophilized powder of each crosslinked scaffold was subjected to FTIR analysis (Nicolet 6700) for characterization. Raman spectra for each group were performed on dry scaffold discs and recorded using a 532 nm laser. The crosslinked scaffold disc was dried overnight to measure the swelling ratio, and the weight of the scaffold disc was measured before and after drying. The swelling ratio of each liver scaffold was calculated according to the following Equation 1:

Swelling ratio (%)=(Ws−Wd)/Wd×100(Ws:weight before drying,Wd:weight after drying)

### Young's Modulus

The elastic modulus of each crosslinked scaffold was obtained using Univert (CellScale, Canada). The crosslinked scaffold was dried for 24 hours and Young's modulus was measured at a loading speed of 1 mm/min. The maximum load was measured by increasing the sample to final breakage. A displacement (mm)—force (N) curve was automatically recorded during the test and then converted to a strain (%)—stress (kPa) curve in consideration of the initial length of the sample.

### In Vitro Collagenase Resistance Test

Collagenase type 1 (MMP-1; Sigma) was used to mimic in vitro collagenase-mediated biodegradation of the scaffold. Each crosslinked scaffold disc was incubated with 100 μg of MMP-1 in a 0.1 M Tris-HCl buffer (pH 7.6) at 37° C. for 2 hours. Thereafter, the reaction was stopped by adding a 10 mM EDTA solution (Gibco, USA). To calculate the weight loss during biodegradation, the weight of each scaffold disc was measured pre- and post-treatment with MMP-1, respectively. The samples were then applied for additional analysis (for example: ninhydrin analysis and quantification of insoluble collagen).

### Ninhydrin Analysis

Free amino acid groups in the crosslinked scaffold were quantified using the ninhydrin reaction after exposure to MMP-1 to estimate the degree of crosslinking. Each scaffold was immersed in a mixture of 1 mL of ninhydrin reagent (N7285, Sigma) and 2 mL of distilled water. The sample was boiled at 100° C. for 10 minutes and then cooled to room temperature. After 3 mL of 95% ethanol was added to each tube, absorbance at a wavelength of 570 nm was measured using a microplate reader (Infinite M200 pro, Tecan, Switzerland).

### Quantification of Insoluble Collagen

After exposure to MMP-1, the insoluble collagen of the crosslinked scaffold was extracted using the Sircol insoluble collagen assay (Biocolor, UK) according to the manufacturer's instructions. The amount of insoluble collagen extracted was measured by reading the absorbance at 550 nm. The obtained results were normalized to the dry weight of each sample. For this purpose, the swelling ratio of each group was measured for normalization as described in Fabrication and characterization of decellularized liver scaffold.

### MMP Activity Assay

A collagenase activity assay kit (ab196999, Abcam) was used to measure the activity of MMP-1. A mixture of different concentrations of NGO (1 μg/mL, 5 μg/mL and 10 μg/mL) with a collagenase cultured at 37° C. for 10 minutes was allowed to react with a collagen matrix. 1,10-phenanthroline was used as a control for collagenase inhibitors. Absorbance at a wavelength of 345 nm was measured in kinetic mode using a microplate reader according to the manufacturer's instructions. The activity of the collagenase was calculated using the absorbance measured minutes and 15 minutes after the enzymatic reaction. To measure the activity of MMP-2 and MMP-9, recombinant MMP-2 (#420-02, Protech, USA) and recombinant MMP-9 (ab155704, Abcam) were activated at 37° C. for 1 hour and 2 hours before being experimented with aminophenylmercuric acetate (APMA), respectively. Activated MMP subtypes were cultured with various concentrations of NGO (1 μg/mL, 5 μg/mL and 10 μg/mL) at 37° C. for 15 minutes. In this case, doxycycline was used as a control for MMP inhibitors. Subsequently, the MMP matrix of the MMP activity assay kit (ab112146, Abcam) was added to the mixture of each group. After the matrix was added using a multiple plate reader (Victor 3, Perkin Elmer, USA), fluorescence intensity was measured at 490/525 nm (Ex/Em) 15 minutes later.

### ¹H NMR and STD-NMR Spectroscopy

The interaction between the catalytic domains of NGO and MMP was investigated at 37° C. using an 850 Hz NMR spectrometer (AVANCEIII HD, Bruker, Germany). All ¹H and STD NMR spectra were measured at 850.22 Hz using a 5 mm TCI cryogenic probe. 100 μg/mL MMP-1 (LS004217, Worthington, USA), MMP-2 (#420-02, Peprotech) and MMP-9 (17104019, Gibco) were cultured with 100 μg/mL NGO at 37° C. NMR analyzes of NGO only, MMP only and MMP mixed with NGO blended in H₂O supplemented with 10% D₂O were then performed. Spectra were collected for off-resonance and on-resonance for STD-NMR analysis. The STD spectrum was obtained by subtracting the on-resonance from the off-resonance spectrum.

### Cell Culture and Aliquot Into Scaffold

HepG2 cells and HUVEC were purchased from ATCC (USA) and stored at 37° C. in a 5% CO₂ incubator. HepG2 cells were cultured in Dulbecco's Modified Eagle Medium (DMEM)-high glucose (Hyclone, USA). HUVECs were cultured in endothelial growth medium-2 (EGM-2, Lonza, Switzerland). All culture media contain antibiotics (100 μg/mL penicillin and 100 μg/mL streptomycin, Gibco) and 10% fetal bovine serum (FBS, Gibco).

To evaluate the biocompatibility of the crosslinked scaffold, HepG2 cells and 2×10⁵ HUVEC were aliquoted into each crosslinked scaffold disc. After 2 hours of static culture, the scaffolds into which the cells of each group were aliquoted were replenished with an appropriate culture medium and maintained for 15 days.

Additional analyses of each scaffold were performed at indicated time points (for example: histological examination, MTT assay, resazurin reduction assay, ELISA, and the like).

### MTT Assay

The metabolic activity of cells in the crosslinked scaffold of each group was measured using MTT assay (tetrazolium-based colorimetric assay). On day 15, the scaffold in which cells were aliquoted on a 12-well plate was washed with PBS and incubated with 10% MTT reagent (Sigma Aldrich) diluted in each culture solution at 37° C. for 4 hours. After 4 hours, the MTT reagent was removed and dimethyl sulfoxide was added. The plate was then placed in a microplate shaker to mix until the cells were sufficiently lysed. The absorbance of MTT formazan at a wavelength of 570 nm was measured using a microplate reader.

### ELISA

To evaluate the biocompatibility of the crosslinked liver scaffold in each group, conditioned media were each harvested from HepG2 cell-aliquoted scaffolds and HUVEC-aliquoted scaffolds. The secretion of albumin and urea in the HepG2 cell-aliquoted scaffold was quantified using Albumin ELISA kit (E80-129, Bethyl Laboratories) and QuantiChrome Urea Assay kit (DIUR-100, BioAssay Systems, USA).

### Ex Vivo Macrophage Polarization in Peripheral Blood Mononuclear Cells (PBMCs)

Human PBMCs were thawed and maintained according to manufacturer's instructions (CC-2702, Lonza). hPBMCs were cultured in RPMI1640 (Gibco) supplemented with 10% FBS for 24 hours for stabilization, and then magnetic-bead activity cell sorting (MACS) was performed using CD14 microbeads (130-050-201, Miltenyi Biotec, Germany). CD14 that was classified as positive was resuspended in the medium. 2×10⁵ CD14+ cells were aliquoted into sterile crosslinked discs (PBS; negative control, GA; positive control, NGO-1, NGO-5 and NGO-10).

After the cells were aliquoted, the scaffolds were incubated with a basal medium supplemented with 20 ng/mL GM-CSF for M1 and 20 ng/mL M-CSF for M2 for 2 days. For M1 polarization, 20 ng/mL IFN-γ (Protech) and 1μg/mL LPS (InvivoGen, USA) were added to the medium and the scaffold was maintained for an additional 5 days. For M2c polarization, the scaffold was further supplemented with 20 ng/mL IL-10 (Protech) and 20 ng/mL TGF-01 (Protech) and maintained for 5 days. On day 7, the scaffold was fixed and immunofluorescence staining and flow cytometry were performed.

### Immunofluorescence Staining

Formalin-fixed, paraffin-embedded tissue was cut into 10 μm sections. Sections of each tissue were deparaffinized with xylene and hydrated with decreasing concentrations of ethanol. The sections were then fixed with 4% formaldehyde in PBS for 10 minutes, and then antigen retrieval was performed with a sodium citrate buffer (pH 6.0, Sigma). After being blocked with 5% normal goat serum (Vector Laboratories, Switzerland) in 0.1% Triton X-100 (Sigma Aldrich) at room temperature for 1 hour, tissue sections were incubated with the following primary antibodies at 4° C. overnight: anti-CD68 (ab31630, Abcam), anti-F4/80 (ab6640, Abcam), anti-CCR7 (ab221209, Abcam), anti-CD206 (ab64693, Abcam), anti-MMP-2 (sc-13594, Santa Cruz Biotechnology, USA), anti-MMP-9(sc-13520, Santa Cruz Biotechnology), anti-TIMP-1(sc-21734, Santa Cruz Biotechnology), and anti-TIMP-2(sc-5539, Santa Cruz Biotechnology). After being washed with PBS containing Triton X-100, sections were incubated with fluorescent dye-conjugated secondary antibodies (Alexa Flour 488- and 594-labeled, Invitrogen) at room temperature for 1 hour. Sections were stained with DAPI (sc-3598, Santa Cruz Biotechnology) for nuclear detection and then mounted with a fluorescent mounting medium (S302380, DAKO, Denmark). Images were visualized with an Eclipse TE 2000 confocal laser scanning microscope (Nikon Japan). CD68⁺ CCR7⁺ cells were considered as M1 polarized macrophages, and CD68⁺ CD206⁺ cells were considered as M2 polarized macrophages. Images were visualized in randomized fields and the ratios of M1-like cells (CD68⁺ CCR7⁺)/CD68⁺ cells and M2-like cells (CD68⁺ CD206⁺)/CD68⁺ cells were each quantified using Image J software.

### Flow Cytometry

To analyze whether the aliquoted CD14+cells were polarized to M1- or M2-like macrophages, cells were extracted from the scaffold using existing protocols for the digestion of mouse liver. First, the scaffold was minced and incubated with 2500 U/mL Collagenase IV (17104019, Gibco) and 1 mg/mL DNase 1 (10104159001, Roche, Switzerland) at 37° C. in a CO₂ incubator for 20 minutes.

After being filtered with a 100 pin nylon cell strainer (352360, Falcon, USA), cells were washed three times with an isolation buffer (4.8% bovine serum albumin and 2 mM EDTA in HBSS) by centrifugation from 4° C. Thereafter, cell pellets were suspended in PBS containing 2% FBS and then incubated with the following antibodies at 4° C. for 15 minutes: CD14 (555397) and CD86 (555659) for M1; CD14, CD163 (563887) and CD206 (555954) for M2. All the antibodies were purchased from BD Bioscience (USA). The cells were washed with PBS and subjected to flow cytometry (FACS Calibur, BD Bioscience). Data was analyzed using FlowJo software (USA).

### In Vivo Transplantation of Crosslinked Scaffold

Sterilized cross-linked scaffold discs (PBS; negative control, GA (glutaraldehyde); positive control, NGO-1, NGO-5, NGO-10) were transplanted into the dorsal subcutaneous pockets of 6-week-old male Balb/c mice (n =20 for each 20 group). Five mice in each group were sacrificed at the indicated time points (days 7, 21, 35 and 60) and transplanted scaffolds for additional analysis were obtained. To examine the induced immune response after transplantation, infiltrated neutrophils and lymphocytes in the transplanted scaffolds were quantified by histological examination.

Immunofluorescence staining was performed to evaluate the polarization of infiltrated macrophages.

The numbers of F4/80 +CCR7⁺ M1-like macrophages and F4/80⁺ CD206⁺ M2-like macrophages were quantified in randomized fields using ImageJ software and the ratio of M1/M2 macrophages was calculated. For serum samples collected from mice, cytometry bead arrays were used to analyze systemic immune responses.

All mouse experiments approved and performed by the Seoul National University Animal Care Committee (SNU-191208-1).

### Statistical Analysis

Statistical analysis was performed and graphs were generated using GraphPad Prism version 5.0. All values used mean±standard deviation. Statistical significance was determined using a two-way ANOVA with a Bonferroni correction, and then an unpaired two-tailed Student's t-test. Values of p<0.05 were considered significant (*p<0.05; **p<0.01; ***p<0.001). All experiments were performed independently at least three times.

### Example 1. Characterization of NGO

To understand the characteristics of the synthesized NGO, a transmission electron microscopy analysis was first performed.

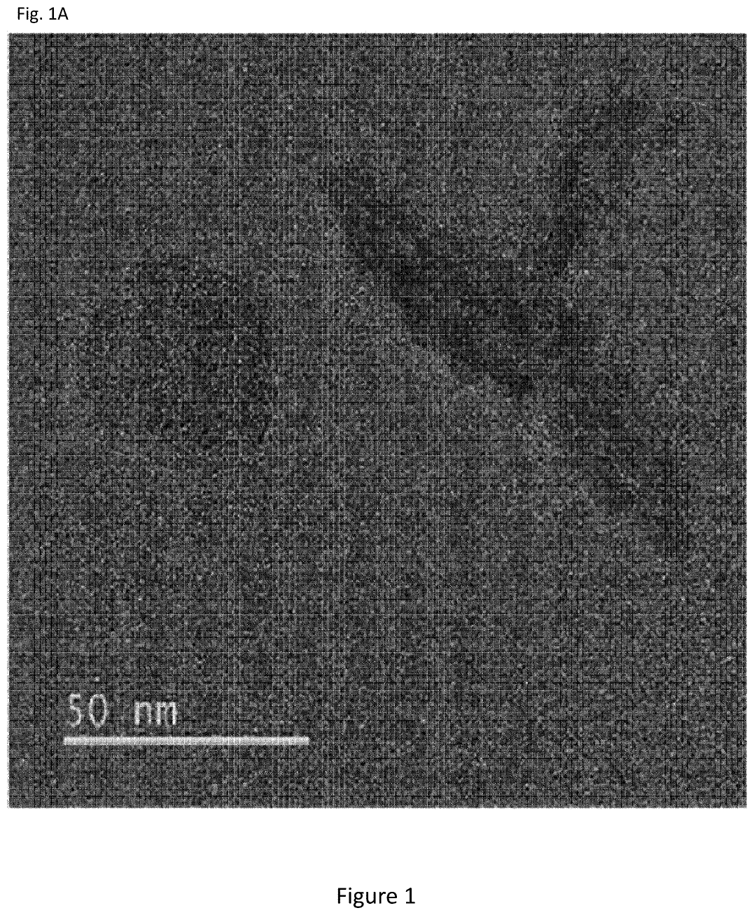

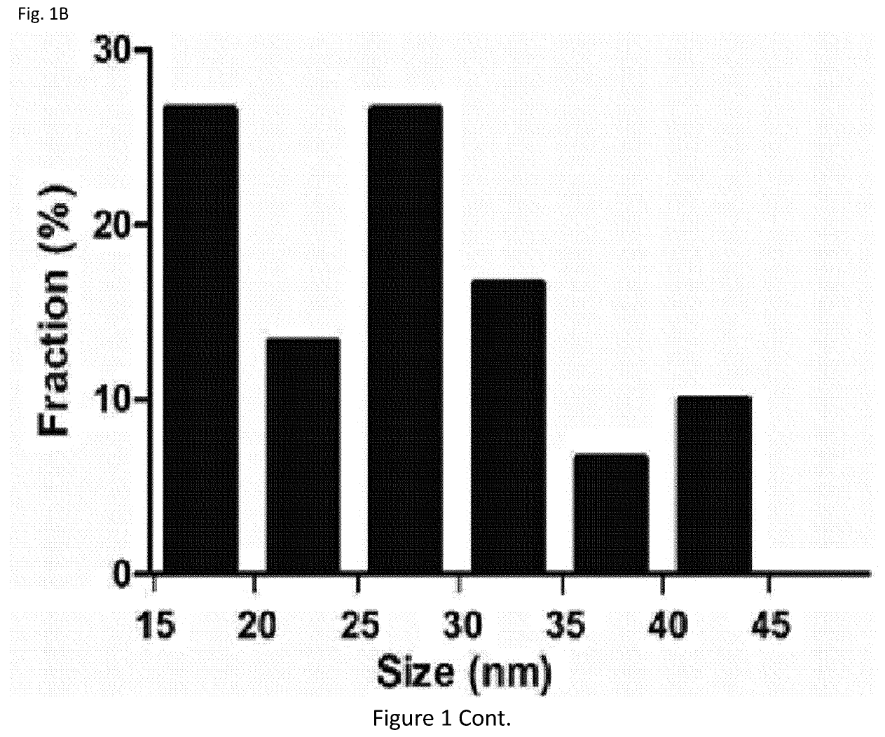

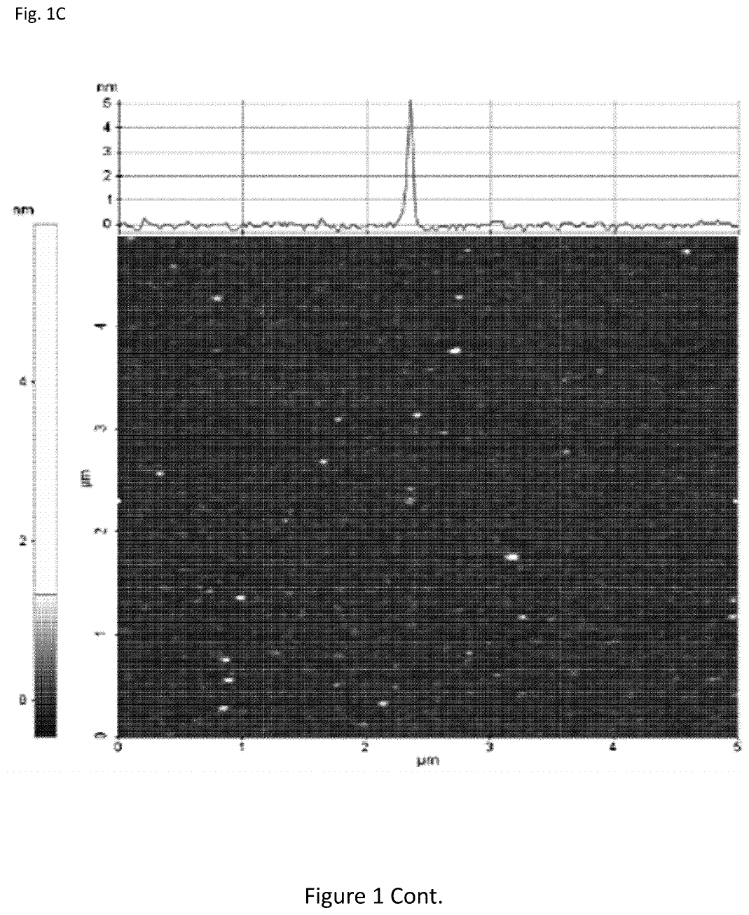

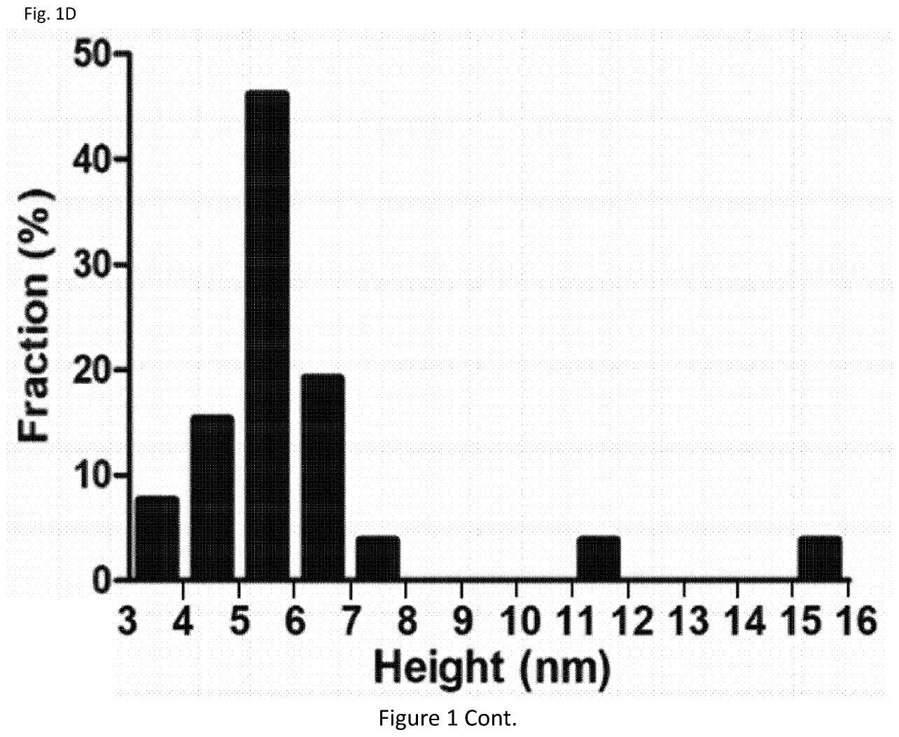

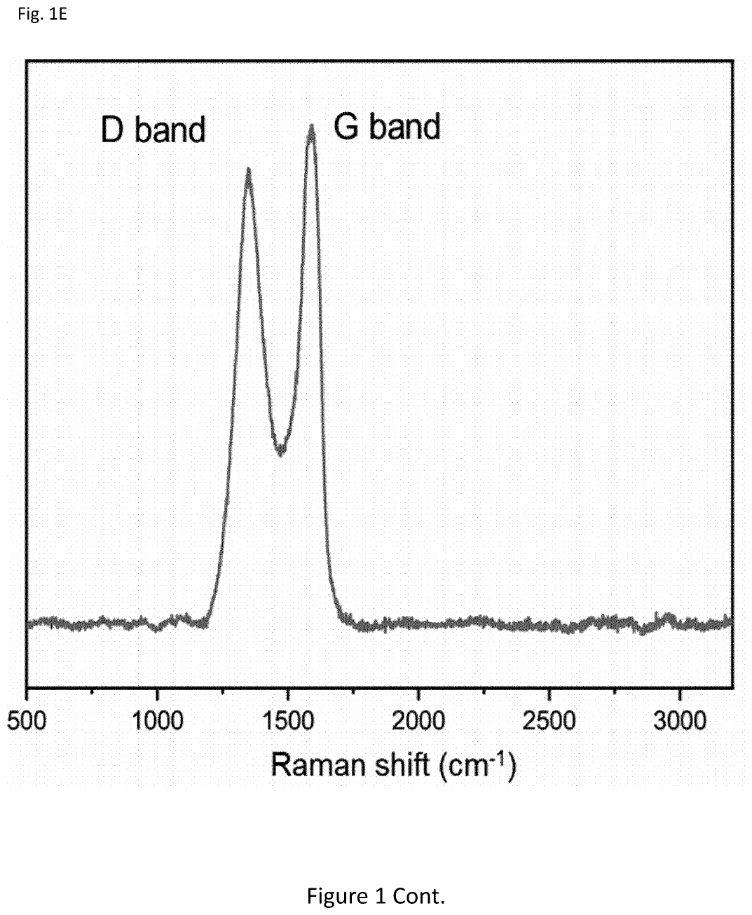

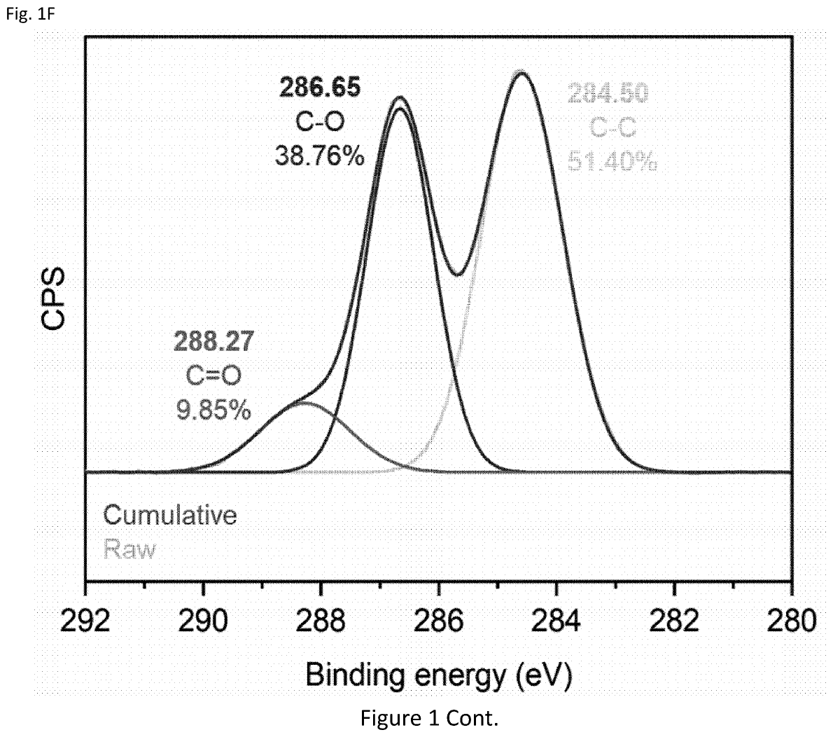

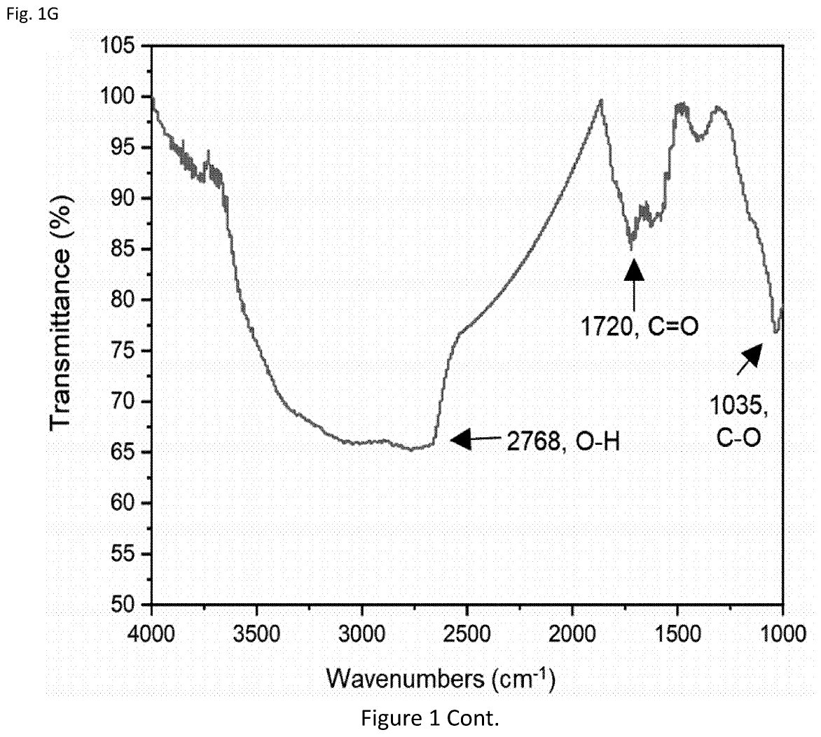

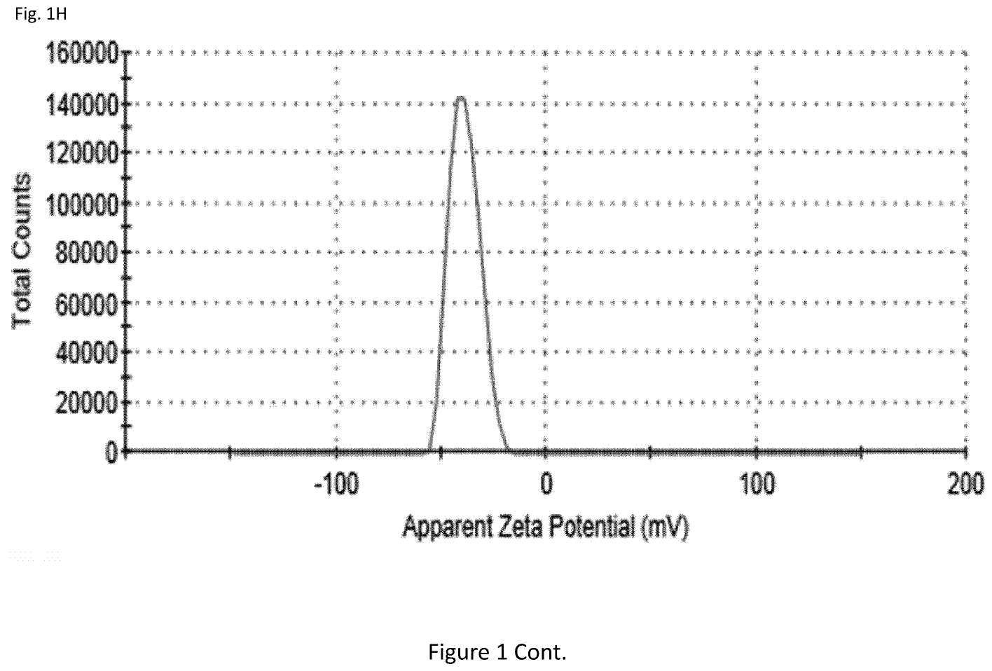

The hexagonal carbon structure of an NGO sheet was observed, and the size of the NGO was shown to be about 30 nm (see FIGS. 1A and 1B). An atomic force microscopy analysis revealed that the atomic thickness of NGO ranged from 3 to 8 nm, suggesting that the NGOs are stacked in less than 10 layers (see FIGS. 1C and 1D). Raman spectroscopy shows peaks at about 1350 cm⁻¹ (D band) and 1580 cm⁻¹ (G band), which are the same results as the characteristic peaks of graphene oxide (see FIG. 1E).

The surface composition of NGO was investigated using X-ray photoelectron spectroscopy.

The C1s spectrum showed three peaks related to C—C (51.4%), C—O (38.76%) and C═O (9.85%) bonds (see FIG. 1F). Fourier transform infrared spectroscopy (FTIR) spectra of the NGO showed peaks at 3428 cm⁻¹, 1720 cm⁻¹ and 1049 cm⁻¹ corresponding to stretching vibrations of OH, C═O and CO bonds, respectively (see FIG. 1G). Such results suggest that hydroxyl, carboxyl and epoxy moieties are present in NGO. Finally, the zeta potential value of −38.6±6.86 mV is the result exhibiting the intermediate dispersion stability of NGO (see FIG. 1H).

### Example 2. Characterization of dECM Liver Scaffold

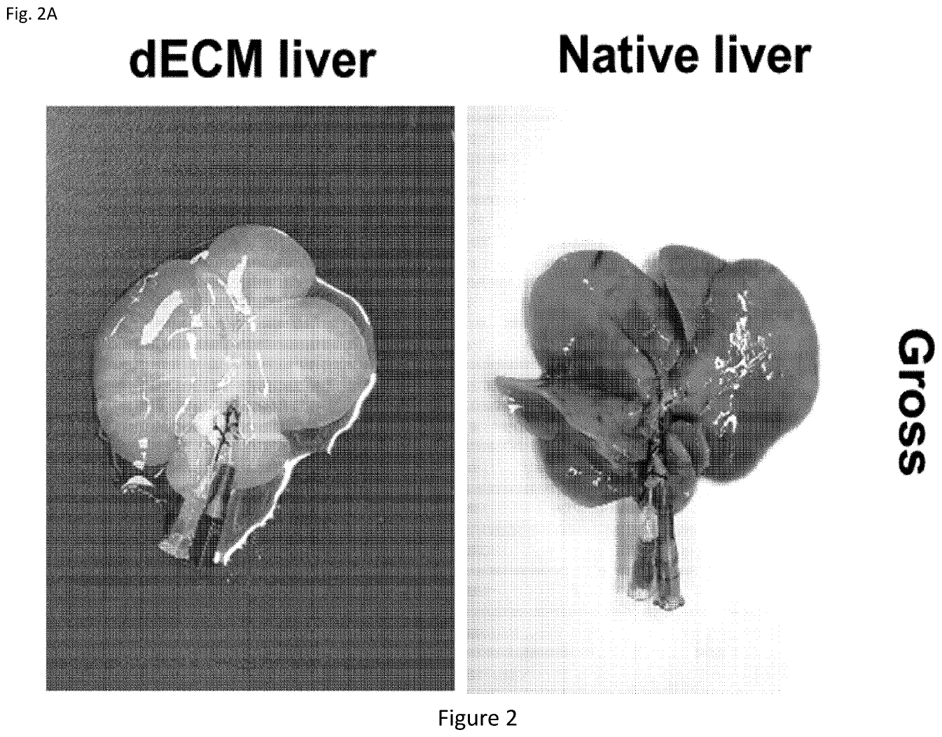

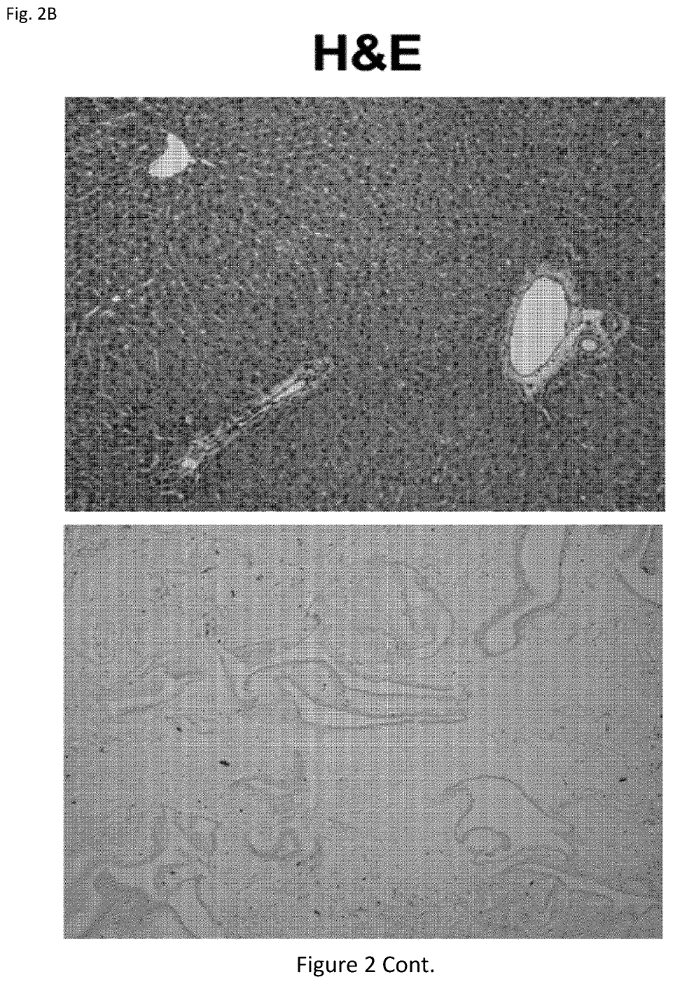

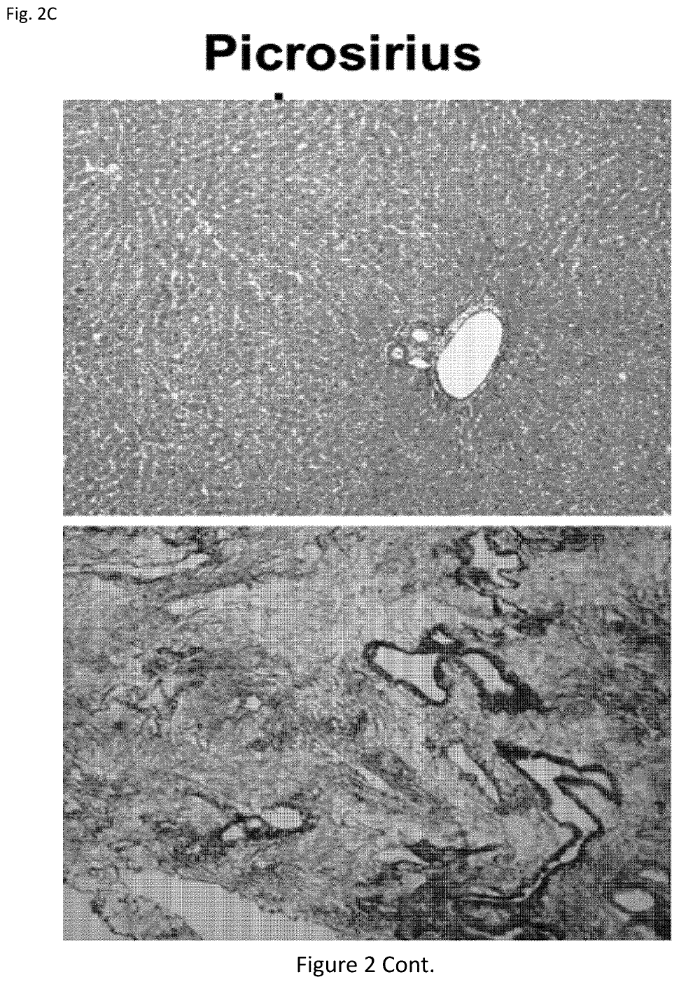

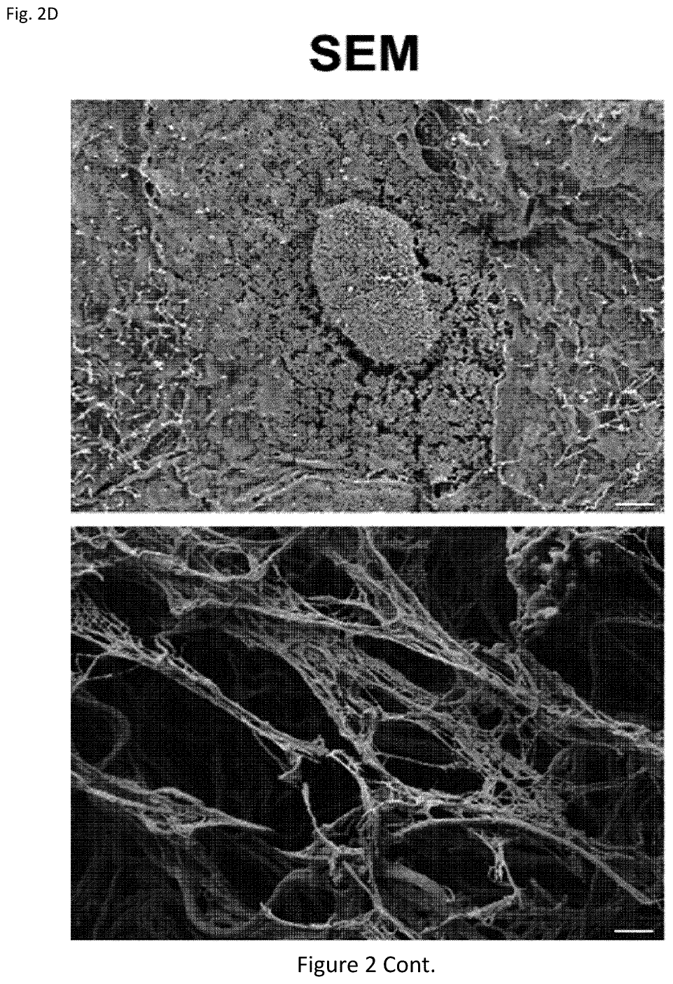

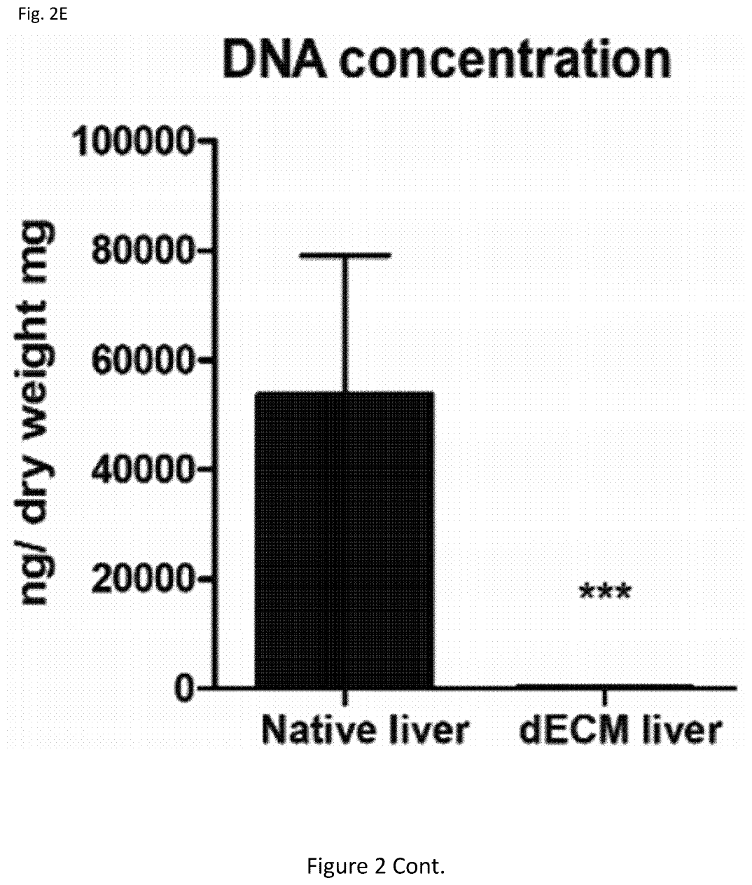

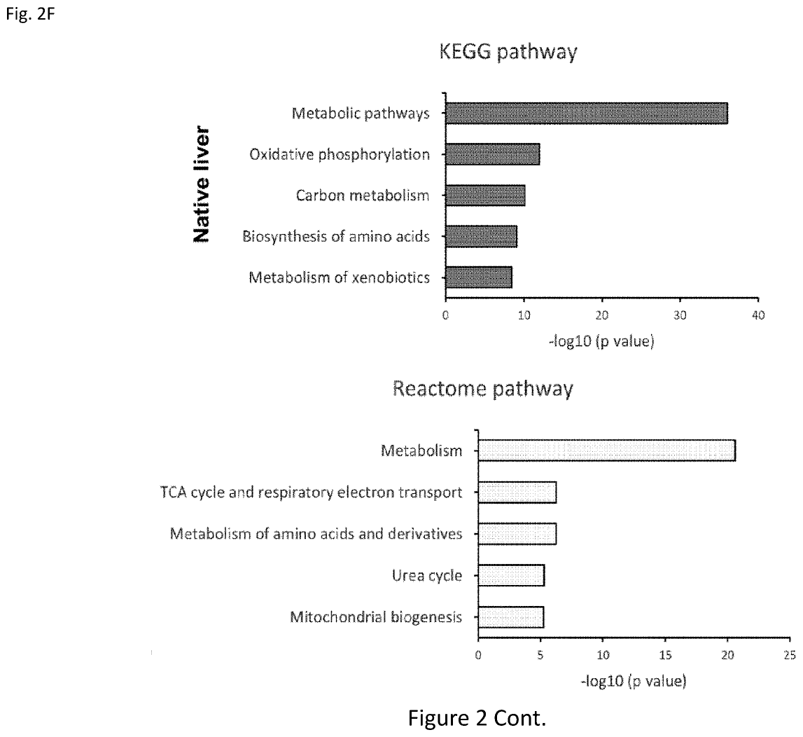

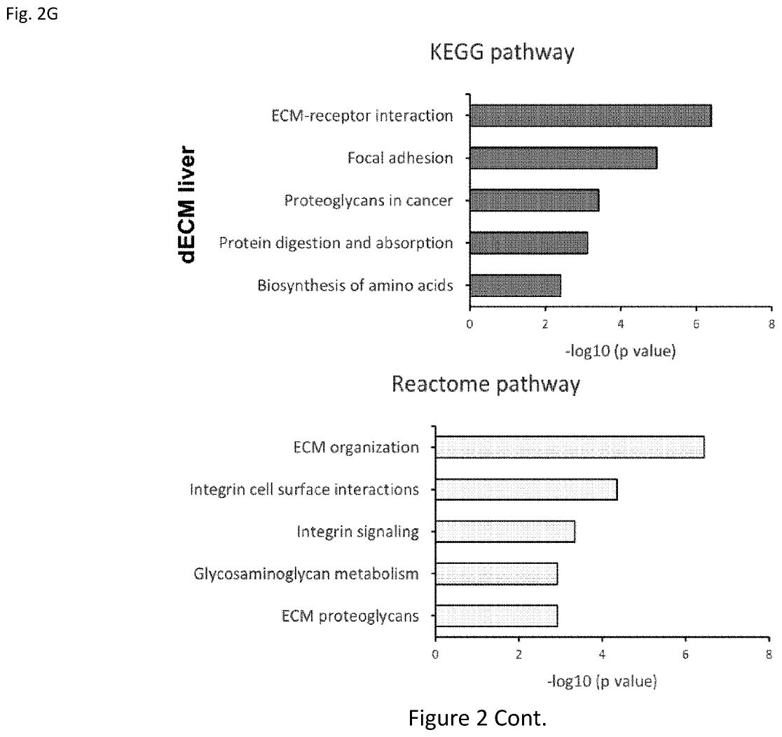

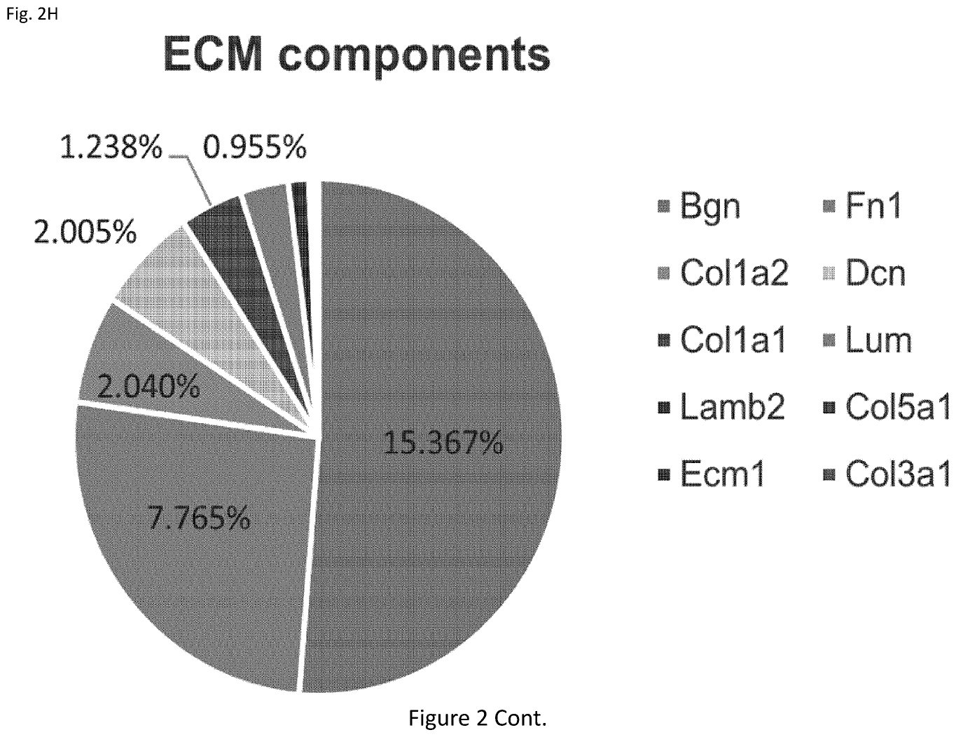

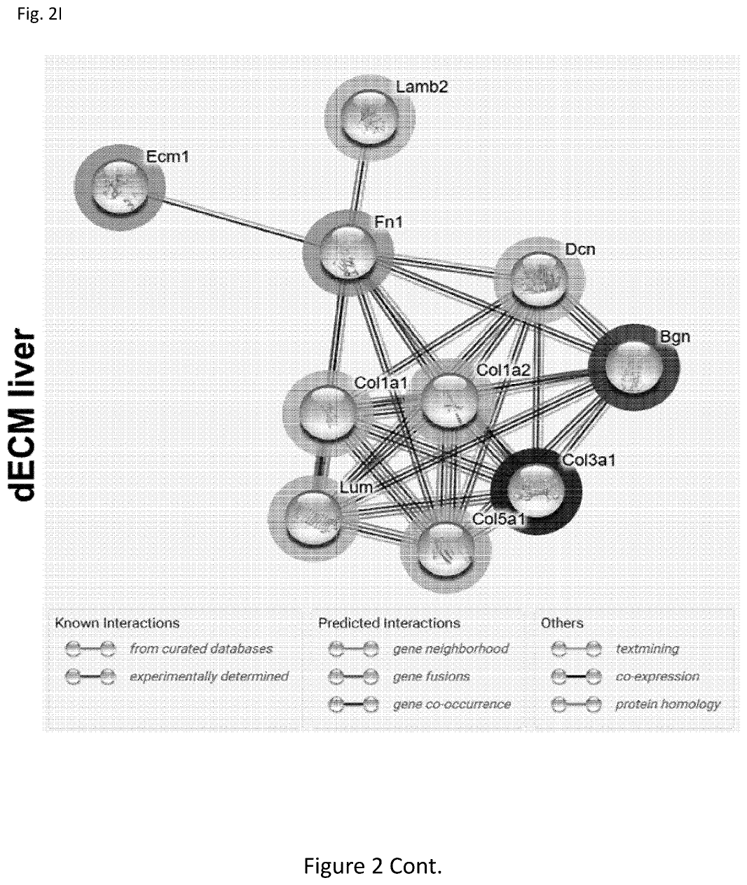

To fabricate a dECM liver scaffold, rat livers were decellularized through perfusion of 0.1% SDS (see FIG. 2A). As a result of structural analysis of the dECM liver scaffold, the preservation of a fibrous structure was shown without existing host cells completely eliminated after decellularization (see FIGS. 2B to 2D).

Liquid chromatography/mass spectrometry (LC/MS) was performed to investigate the proteomic profile of liver tissue. Consequently, as a result of KEGG pathway and Reactome pathway analysis of the top 100 proteins, it was shown that native liver was usually associated with metabolism, whereas in dECM liver, most of the proteins associated with ECM tissue are at the top (see FIGS. 2E and 2F). In particular, biglycan, fibronectin, and type 1 collagen were identified as major ECM components of dECM liver scaffold (see FIGS. 2G and 2H).

### Example 3. Fabrication of NGO Crosslinked dECM Liver Scaffold

For crosslinkage, the dECM liver scaffold was perfused with PBS (CTL, negative control), 0.625% glutaraldehyde (GA, positive control) and various concentrations of NGO (1 μg/mL; NGO-1,5 μg/mL; NGO-5, and 10 μg/mL; NGO-10).

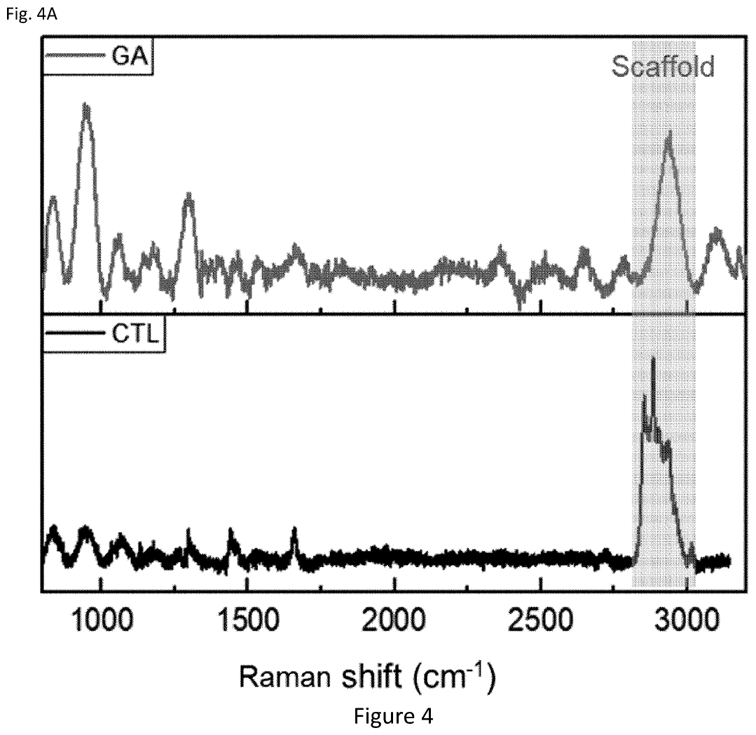

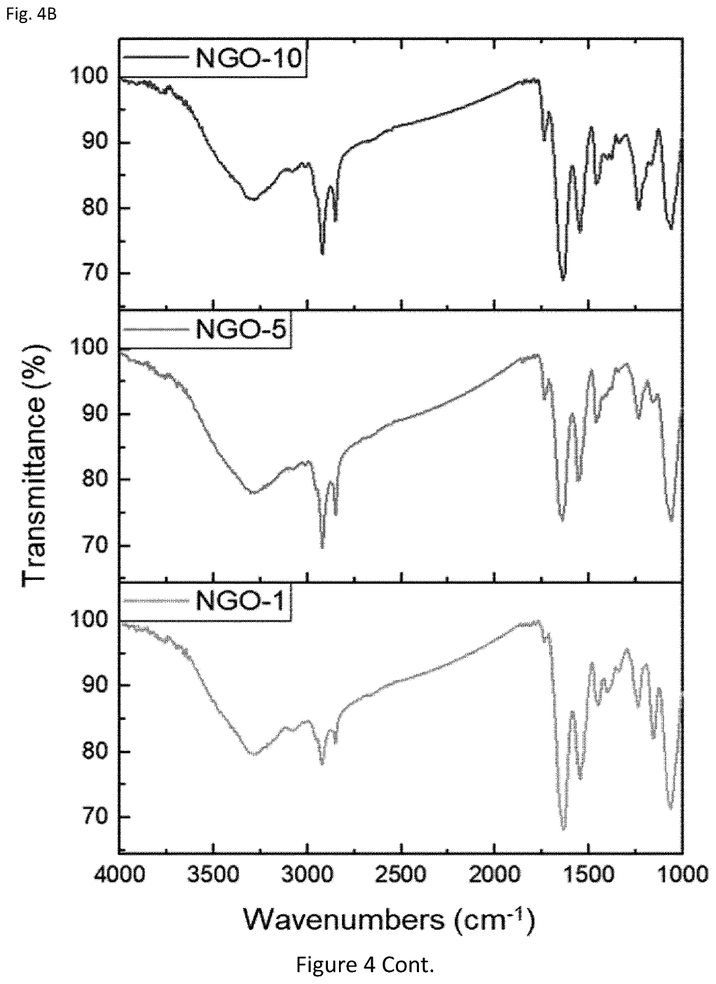

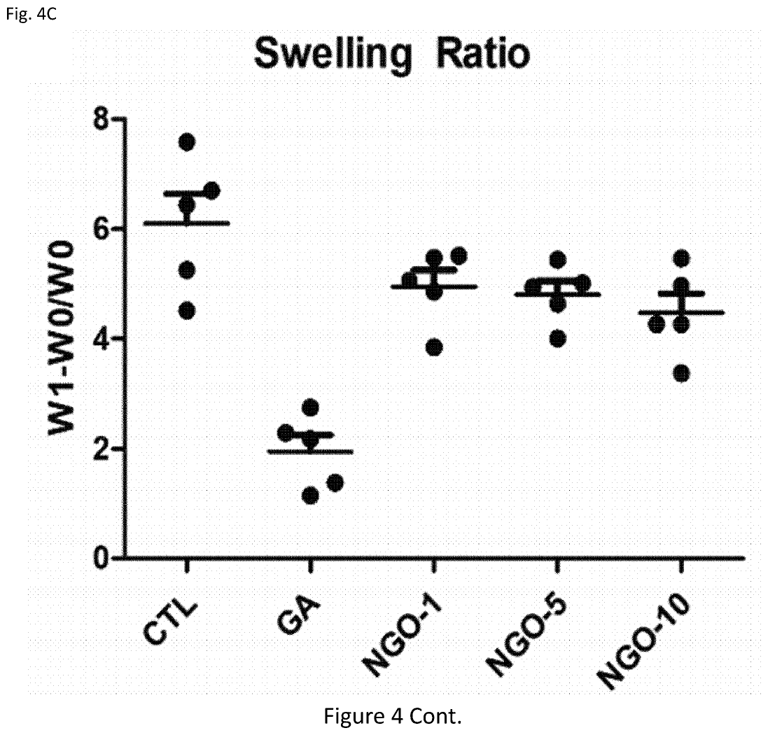

As a result of scanning electron microscopy (SEM) analysis, it was shown that NGO was bound to the fibrous network of the ECM in the crosslinked scaffold (see FIG. 3A). As a result of Raman spectroscopy analysis, the peaks of the NGO-crosslinked scaffold appeared not only at 2930 cm⁻¹, which is the dECM scaffold peak, but also at 1350 cm⁻¹ (D-band) and 1580 cm⁻¹ (G band), which are the characteristic peaks of NGO regardless of NGO concentration. These results indicate the presence of NGO in the DECM scaffold after crosslinkage (see FIG. 3B). As expected, no NGO peaks were detected in the GA-crosslinked scaffold (see FIG. 4A).

Next, FTIR analysis was performed to verify the chemical composition characteristics of NGO and scaffold. In the NGO-5 group, peaks were exhibited at 1720 cm⁻¹, 1157 cm⁻¹ and 1060 cm⁻¹ indicating the hydrocarbon moieties of NGO (see FIG. 3C).

Meanwhile, the transmittance of the 3085 cm -1 peak assigned to a primary amine group decreased in the NGO-5 scaffold compared to the CTL scaffold, suggesting that the carboxyl group of NGO binds to free amine groups present in the macromolecules of the dECM scaffold to form a peptide bond during crosslinkage. Further, scaffolds crosslinked to different concentrations of NGO showed exactly the same pattern of chemical bonding (see FIG. 4B). Based on the above results, a dECM liver scaffold to which NGO was bound was successfully developed.

Thereafter, it was hypothesized that NGO crosslinkage could improve the physical stiffness of the dECM scaffold.

Thus, it was confirmed that the elastic modulus of the NGO-crosslinked scaffold was similar to that of the GA-crosslinked scaffold and much higher than that of the CTL scaffold (see FIGS. 3D and 3E).

In addition, the maximum tensile strength identified as the maximum pressure that the scaffold can withstand was found to be higher in the NGO-crosslinked scaffold than in the CTL scaffold (see FIG. 3F).

From the above results, it was confirmed that the mechanical properties of the dECM scaffold were strengthened through NGO crosslinkage.

### Example 4. dECM Scaffold In Vitro Degradation Protection Effect of NGO Crosslinkage

Various types of cells including macrophages, neutrophils and fibroblasts were harvested after transplantation of the dECM scaffold through chemotaxis, and consequently, such cells secreting proteases may contribute to scaffold degradation. Therefore, whether NGO-crosslinked scaffold can protect the ECM structure from enzymatic degradation is an important subject. In consideration of the proteomics data, it was shown that type 1 collagen, and biglycan, which is known to coexist with type 1 collagen and involved in collagen matrix assembly, constitute the main structural elements of the dECM liver scaffold (see FIG. 2I). Thus, metalloproteinase (MMP)-1, also called collagen degrading enzyme-1, was used to reproduce the degradation of the in vitro scaffold.

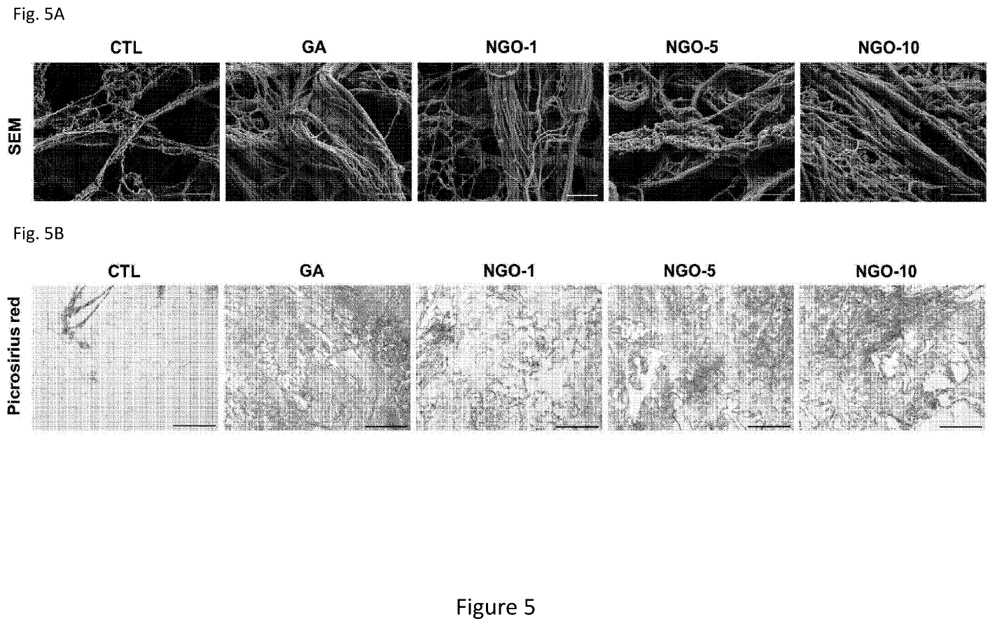

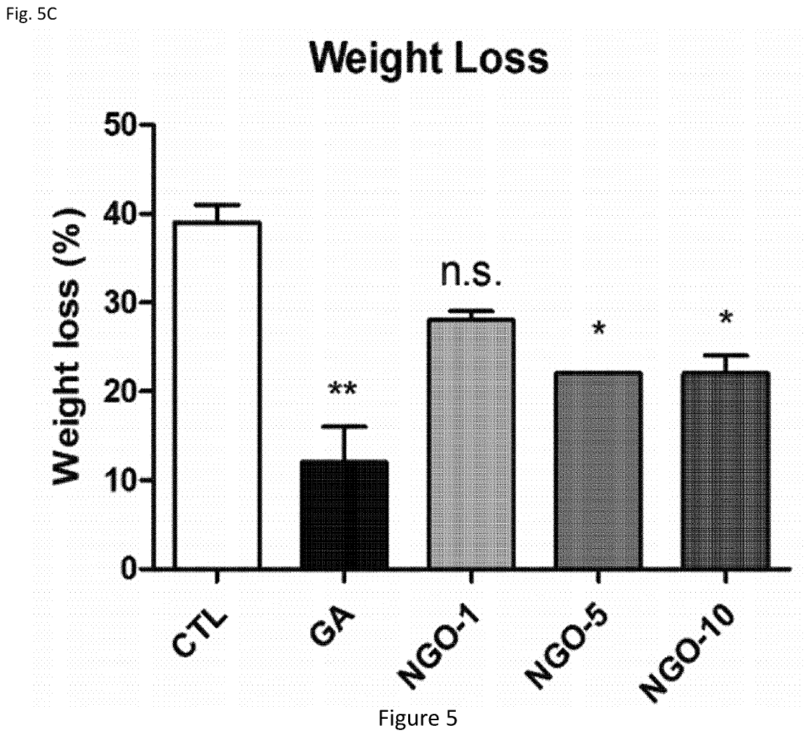

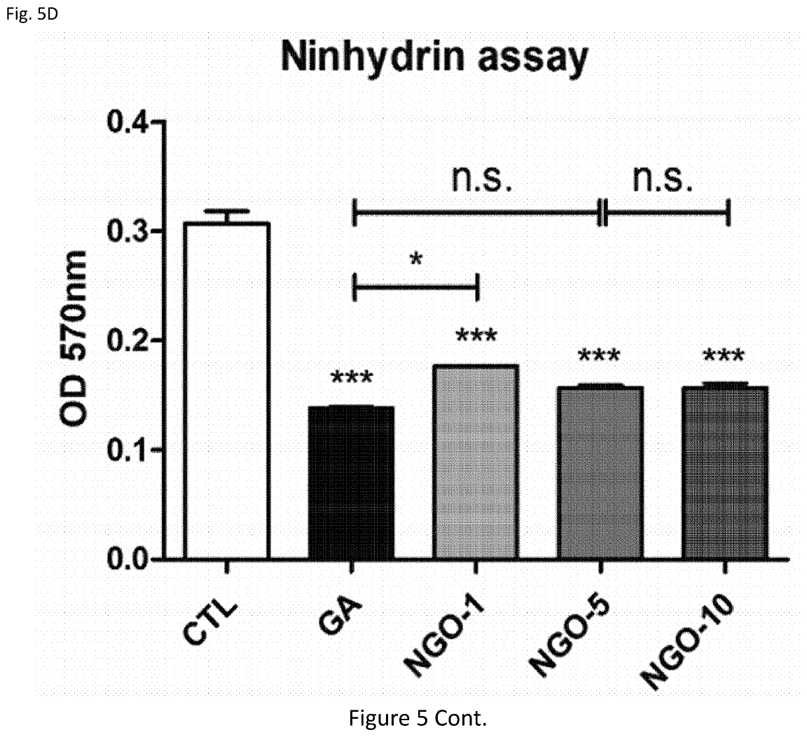

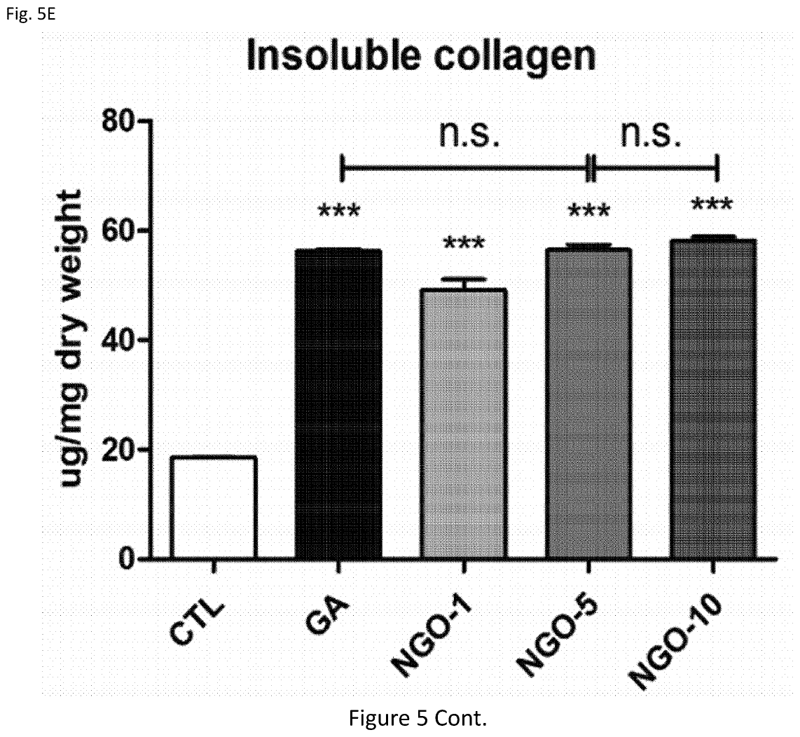

As a result of SEM analysis, it was shown that the microstructure of the control (CTL) scaffold exposed to MMP-1 was disrupted, leaving only thin ECM fibers, whereas the microstructure of the NGO- or GA-crosslinked scaffold is not reduced by treatment with a collagen degrading enzyme (see FIG. 5A). These results were found to be consistent with Picrosirius red staining showing strong collagen dissociation in CTL scaffolds (see FIG. 5B).

Furthermore, after MMP-1 treatment, the weight loss of each scaffold was significantly reduced in the presence of a crosslinking agent compared to the PBS control (see FIG. 5C).

Further, the degree of crosslinking of each scaffold was determined by performing ninhydrin analysis.

As a result, it was confirmed that the concentration of free amino acids not involved in covalent bond formation during crosslinkage was remarkably reduced in the NGO-crosslinked scaffold to a level as low as in the GA-crosslinked scaffold (see FIG. 5D). Such results could also be confirmed in the NGO-crosslinked scaffold showing reduced free amine groups compared to the CTL scaffold by FTIR analysis. Next, the residual amount of insoluble collagen, which is a crosslinked form of collagen fibers, was measured in the crosslinked scaffolds exposed to collagen degrading enzymes and normalized to the dry weight of each scaffold (see FIG. 4C). It was found that the amount of insoluble collagen retained in the NGO group was similar to the amount of GA-crosslinked scaffold (see FIG. 5E).

That is, it was demonstrated that NGO-crosslinked scaffolds effectively suppressed collagen degrading enzyme-mediated degradation and alleviated the loss of ECM components compared to CTL scaffolds.

In summary, the above results suggest that the crosslinkage of NGO strengthened the resistance of dECM scaffolds to enzymatic degradation.

### Example 5. Suppressive Effect of NGO on MMP Activities Through Direct Interaction

It was additionally investigated whether NGO has a mechanism that it could protect against damage to dECM scaffolds. Since it was previously known that graphene could affect the activity of enzymes through enzyme immobilization, the interaction between NGO and activated MMPs (MMP-1, MMP-2 and MMP-9) involved in cleaving the major extracellular matrix components of the dECM scaffold was investigated using nuclear magnetic resonance (NMR).

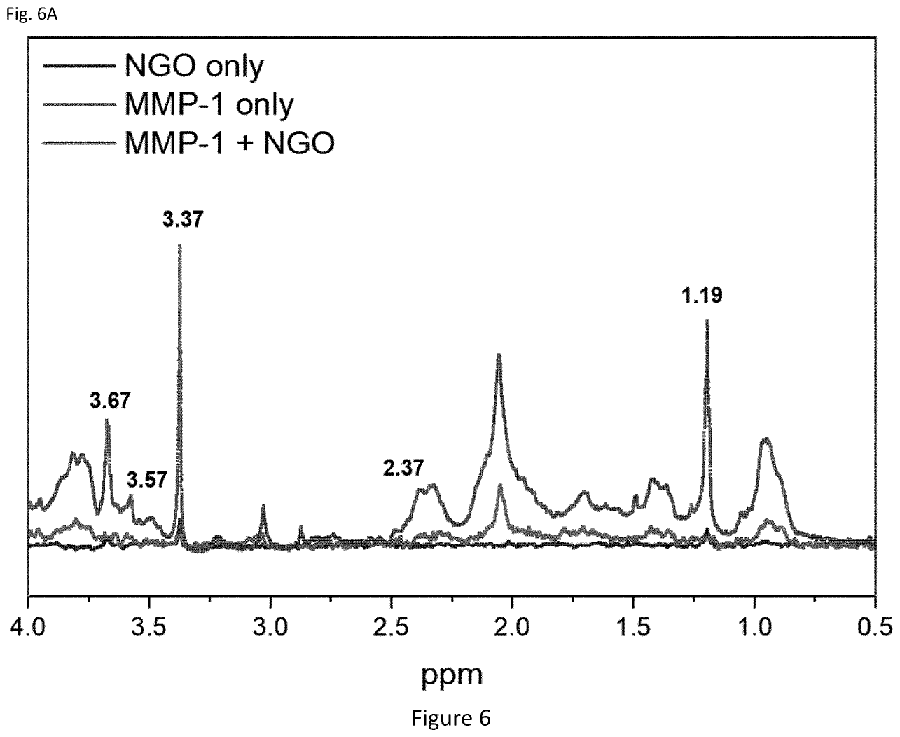

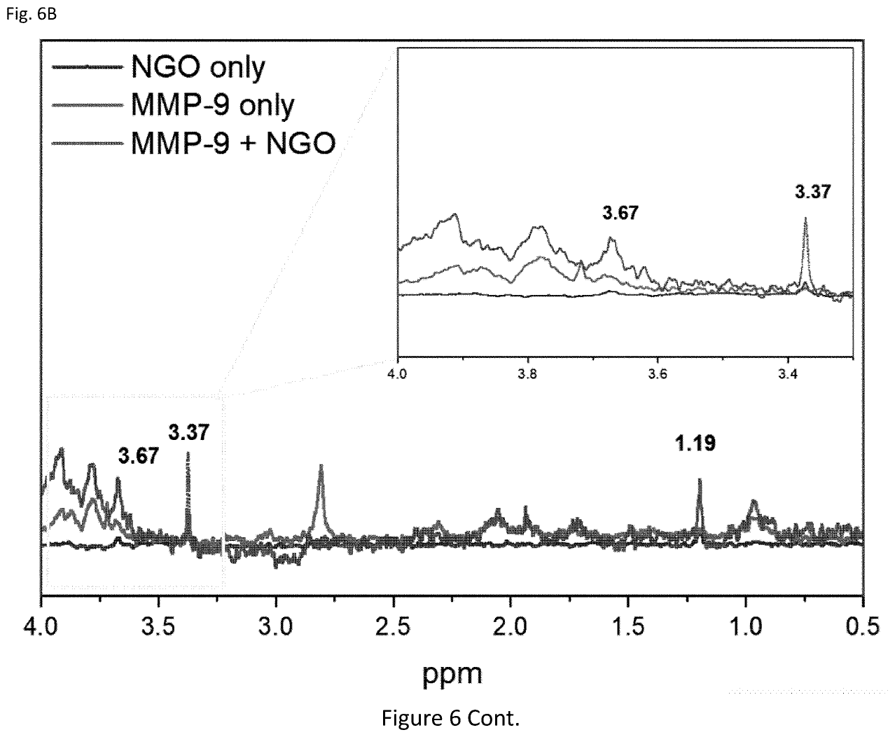

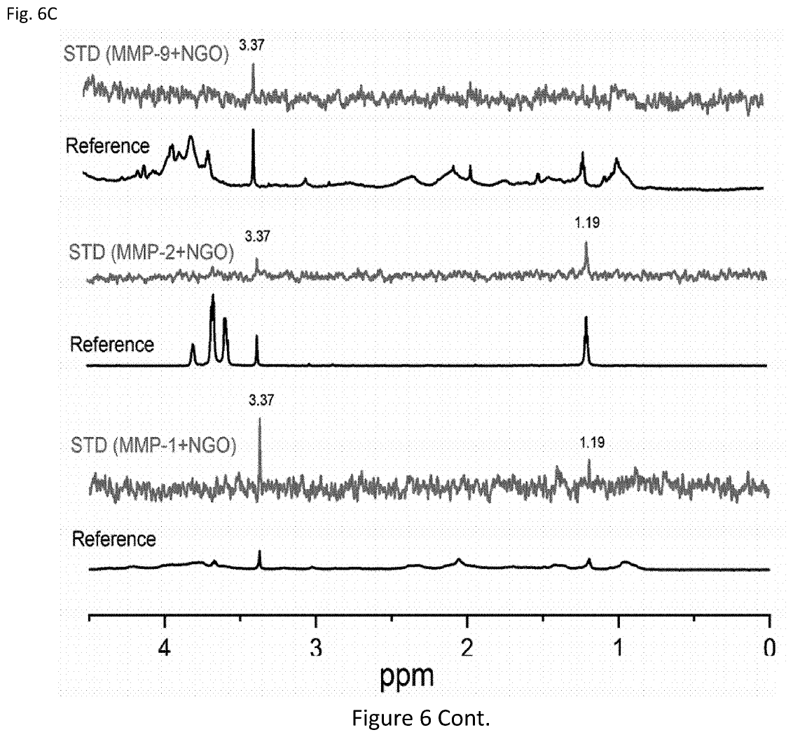

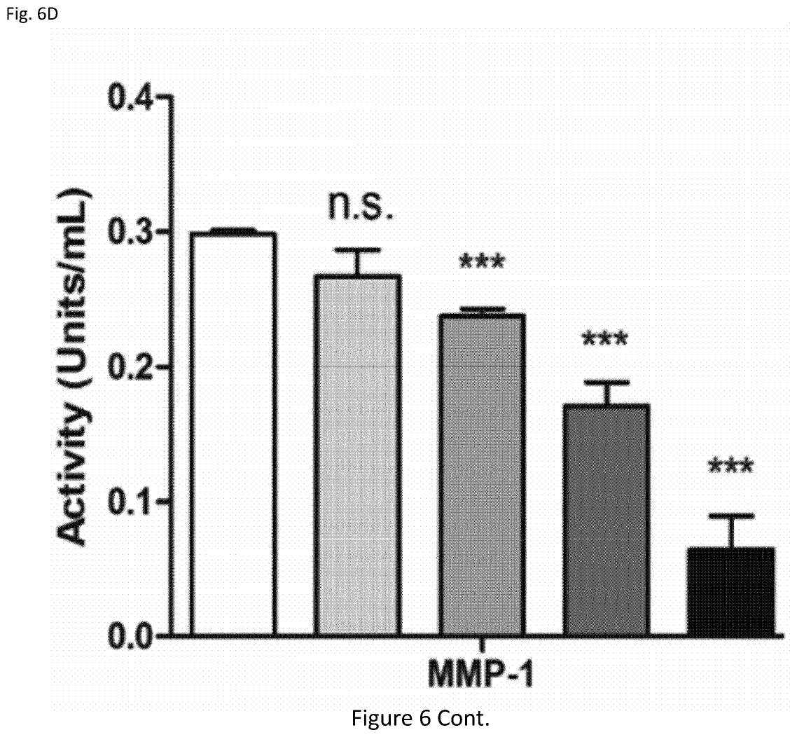

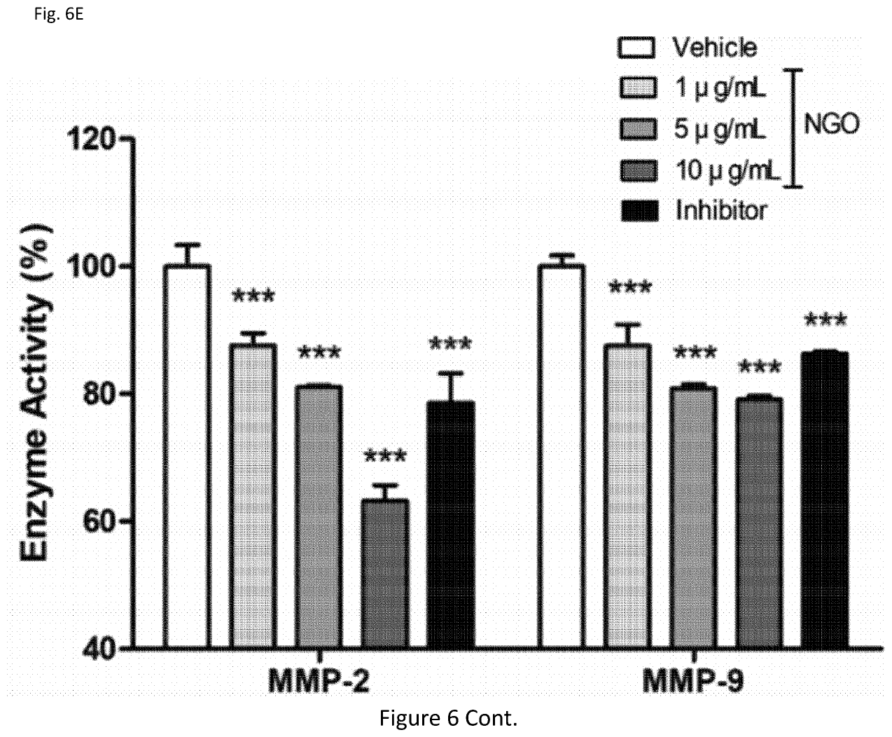

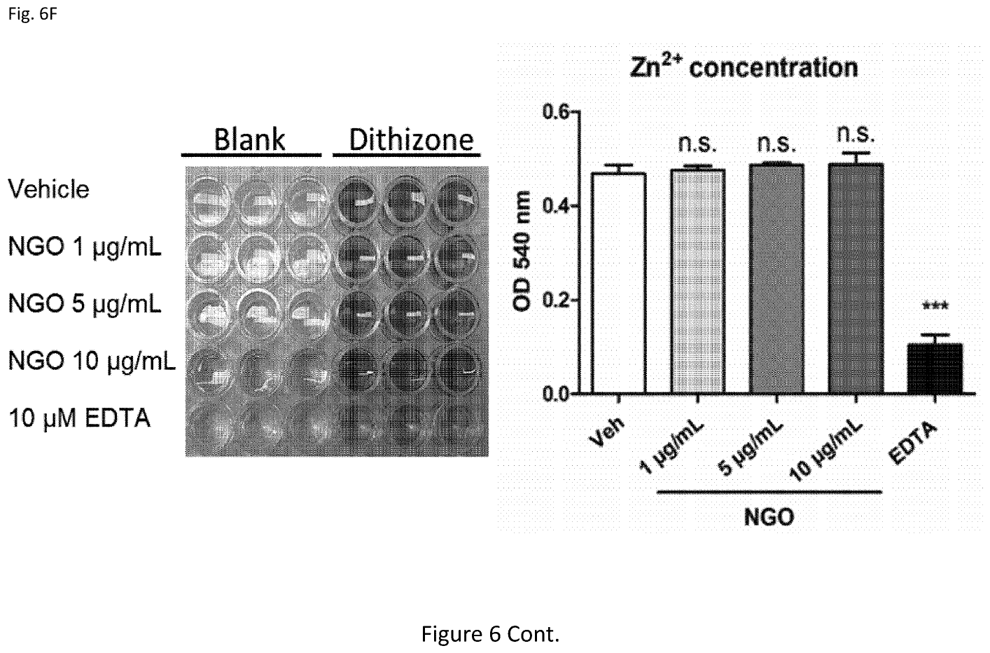

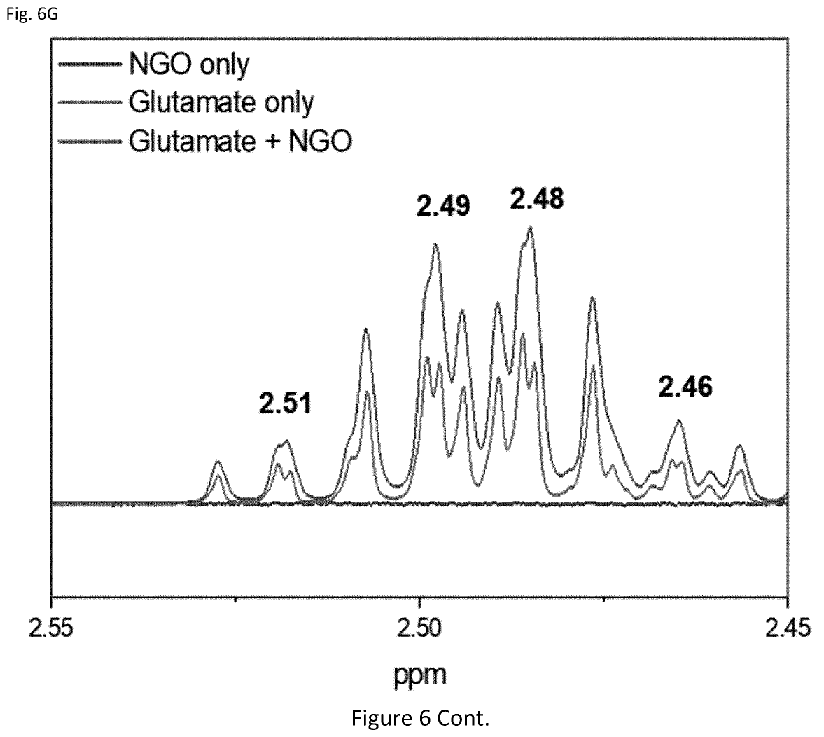

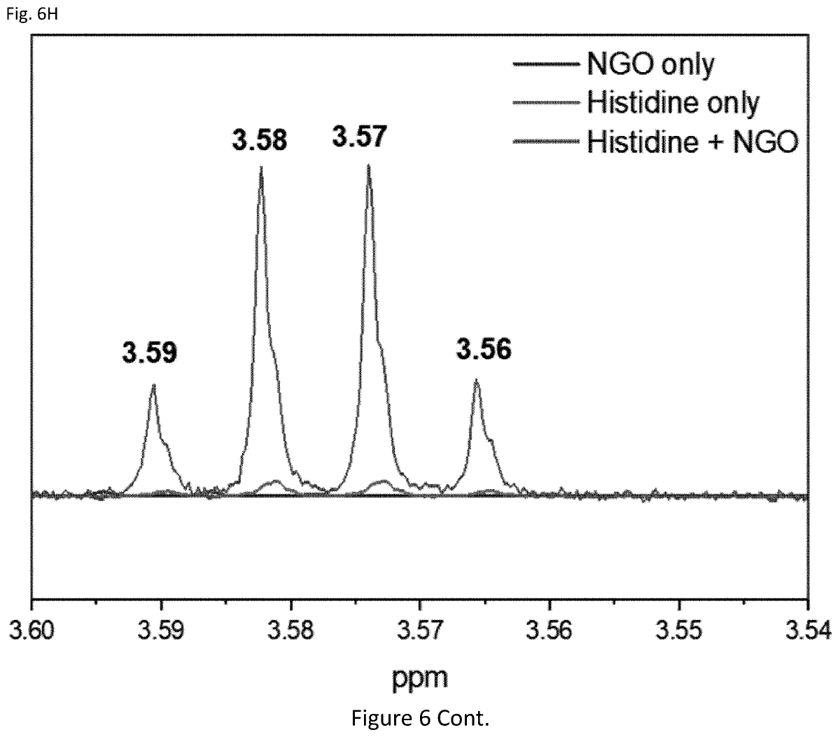

As illustrated in FIGS. 6A and 6B, as a result of ¹H NMR analysis, the chemical shift of MMP could be confirmed in the presence of NGO.

As illustrated in FIG. 6C, NGO and MMP incubated with NGO could confirm peaks at 1.19 and 3.37 ppm on the ¹H NMR spectrum, which is a result suggesting that NGO interacts directly with MMP. In particular, the interaction between MMP and NGO tended to occur in the same region (1.19, 3.37 ppm) regardless of MMP subtypes, suggesting that NGO can bind to the catalytic domain, which is a highly conserved region among diverse types of MMP.

As illustrated in FIGS. 6D and 6E, NGO significantly decreased the catalytic activity of MMP-1, MMP-2 and MMP-9 in a dose-dependent manner. That is, it was confirmed that NGO can suppress the enzyme activity of MMP by directly binding to the catalytic domain of MMP.

Meanwhile, the proteolytic mechanism requires a nucleophilic water molecule in which a conserved catalytic zinc ion and a conserved zinc-binding motif (HExxHxxGxxH) are planked. Three histidine (H) residues participate in the structural coordination of catalytic zinc, whereas the glutamate (G) residue forms hydrogen bonds with water and plays a general acid/base role in the catalytic process.

Thus, the zinc chelation effect of NGO was investigated using dithizone.

As illustrated in FIG. 6F, it was confirmed that NGO could not directly chelate zinc compared to EDTA, which is a zinc chelator.

Thus, it was confirmed whether NGO affected the zinc-binding motif.

As a result, as illustrated in FIGS. 6G and 6H, it was confirmed that NGO interacts with glutamate and histidine, which are components in the zinc-binding motif.

Glutamate residues of MMP are involved in nucleophilic attack on the carbonyl carbon of a matrix for peptide cleavage, but NGO bound to glutamate residues is likely to cause the catalytic dysfunction of MMP by modifying a proton transfer process after nucleophilic attack through proton dissociation of oxygen-containing functional groups.

Furthermore, histidine residues also assist catalytic zinc for structural stability, but NGO bound to histidine residues causes 3D conformational changes and disrupt the spatial regulation of MMP, leading to the catalytic dysfunction of zinc. Accordingly, NGO is expected to be able to suppress the enzyme activity of MMP through direct binding to zinc-harboring motifs.

Therefore, it is shown that NGO suppresses proteolysis by MMP enzymes and may affect MMP-mediated pathophysiological responses.

### Example 6. High Biocompatibility of NGO Crosslinked Scaffold

Human hepatocytes and endothelial cells were used to evaluate biocompatibility, which is an essential element for practical liver tissue engineering applications.

HepG2 cells and human umbilical vein endothelial cells (HUVECs) were aliquoted into crosslinked scaffolds and maintained for 14 days.

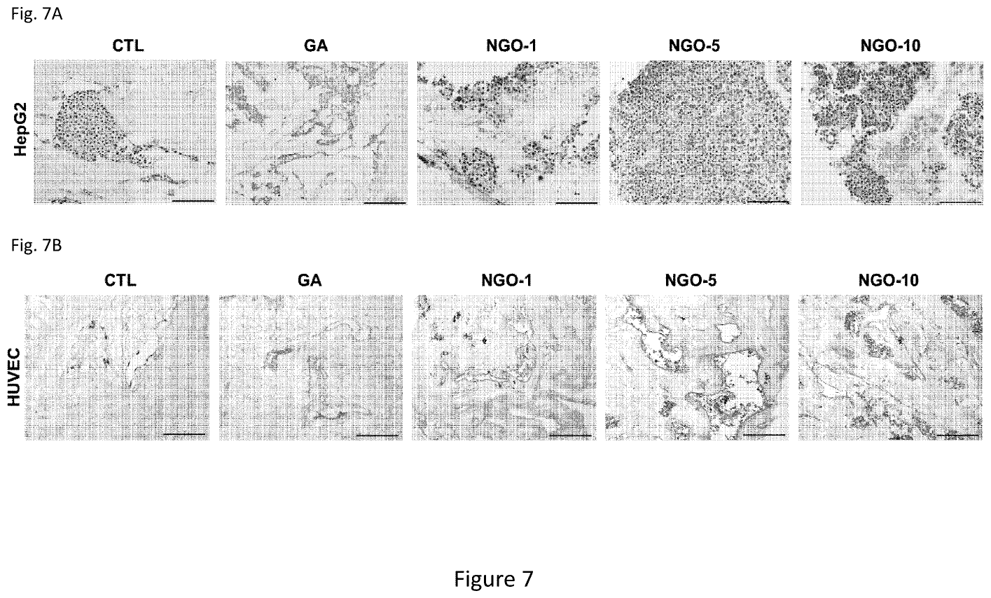

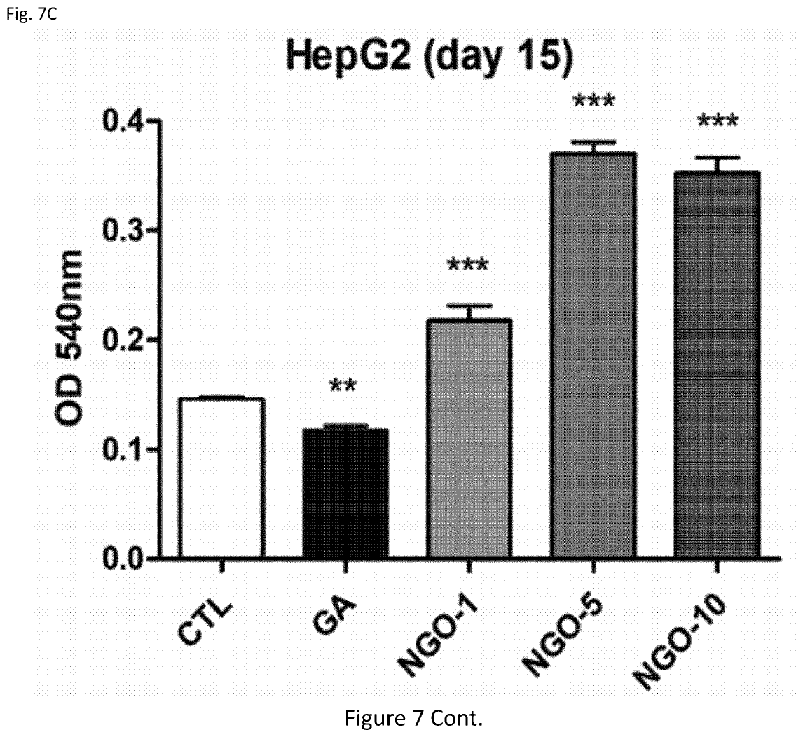

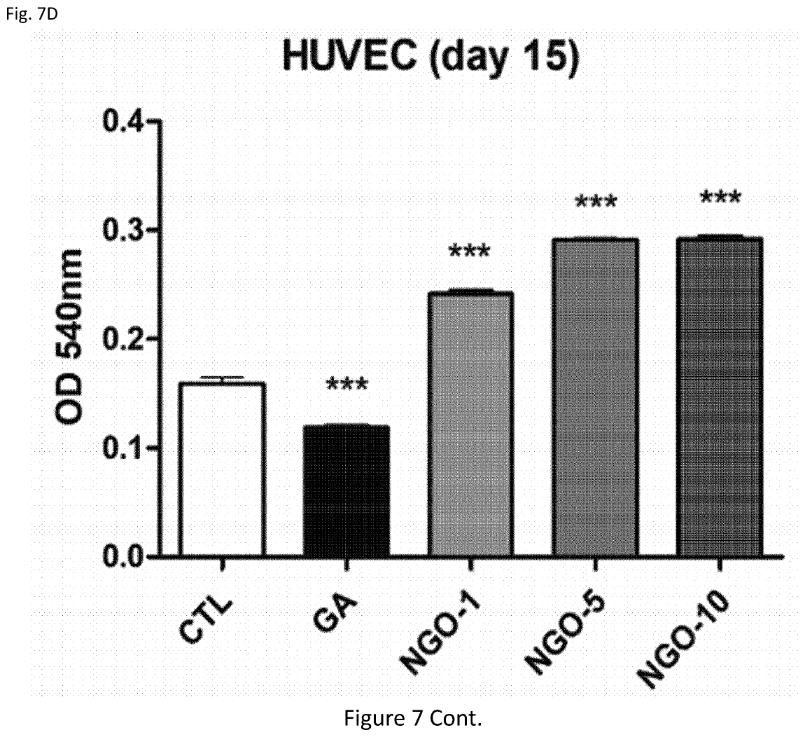

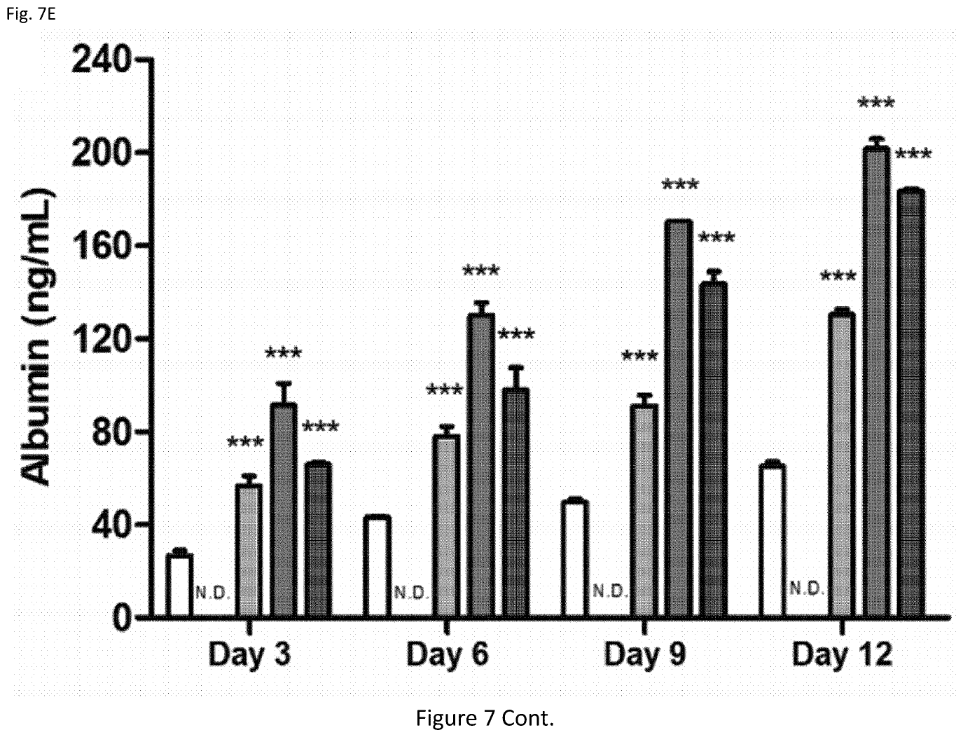

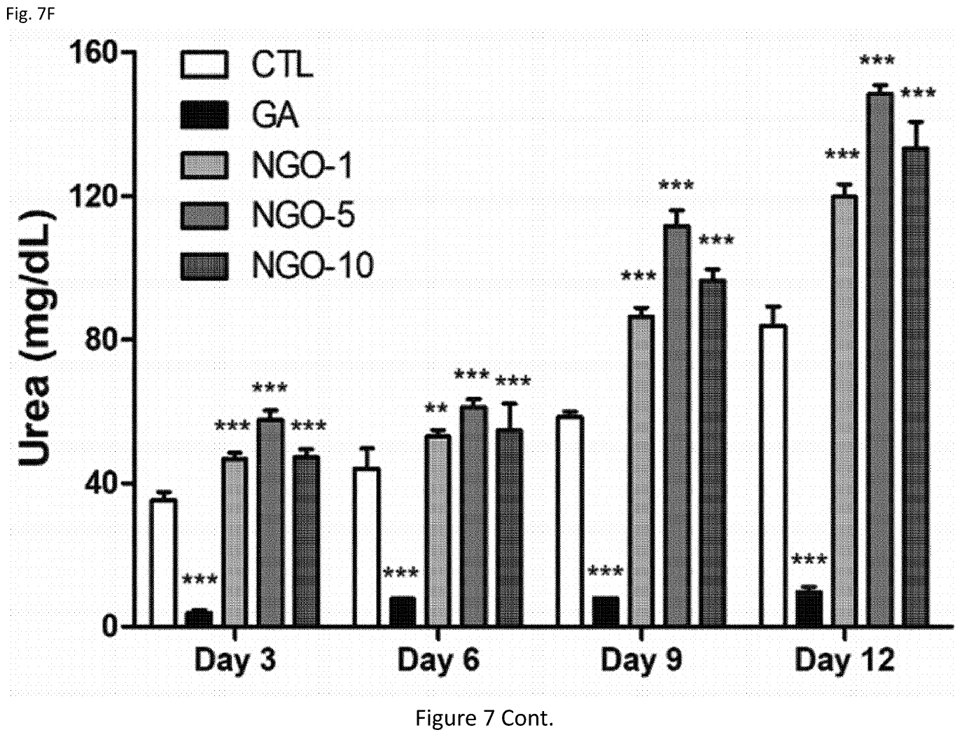

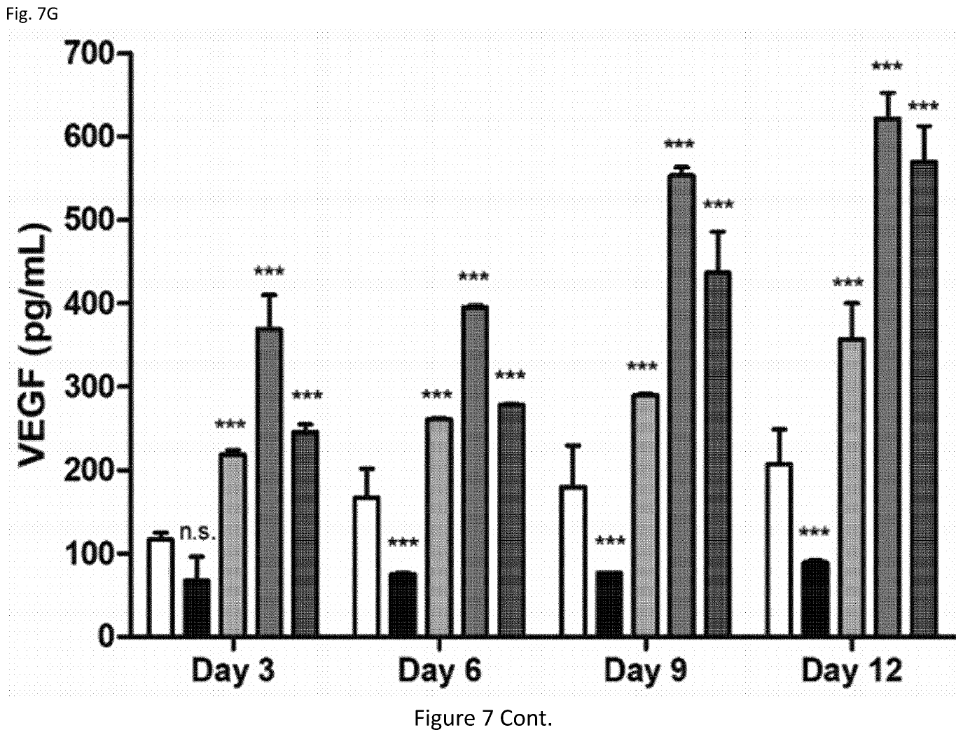

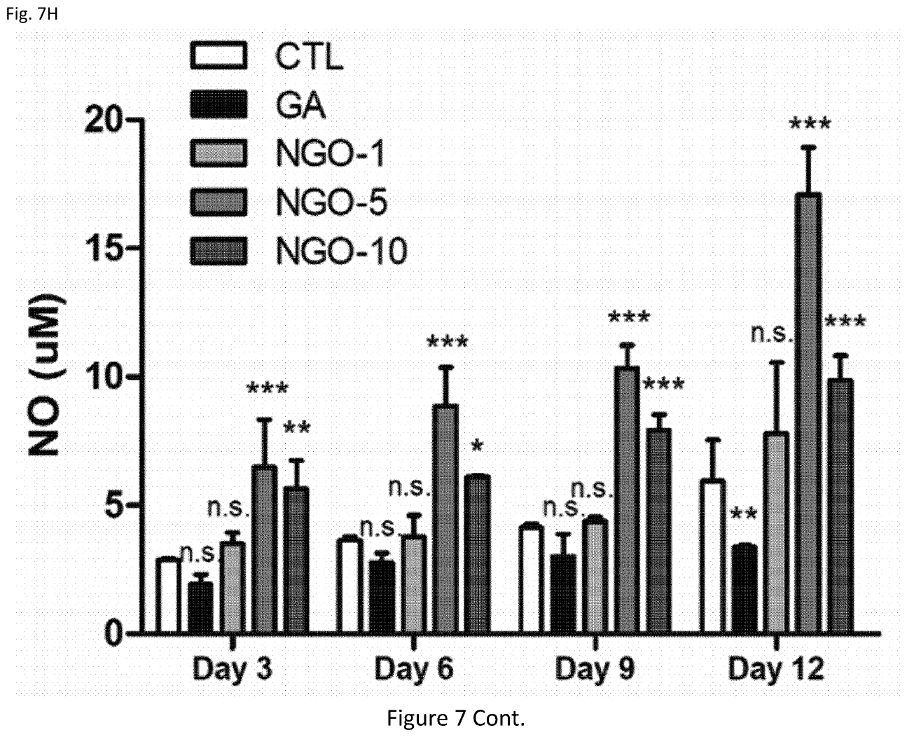

From the results obtained from H&E staining and MTT analysis on day 15, it was confirmed that scaffolds crosslinked to NGO possessed higher viability and proliferating cell populations compared to the other groups (see FIGS. 7A to 7D).

Therefore, liver-specific functions including albumin and urea secretion and glycogen synthesis were remarkably increased in the NGO-crosslinked group into which HepG2 cells were aliquoted compared to the other groups (see FIGS. 7E and 7F).

Further, it was found that VEGF and NO secretion, which are the characteristic of the vascular system, was increased sharply in the HUVEC-aliquoted NGO-5 group (see FIGS. 7G and 7H). In contrast, since GA-crosslinked scaffolds showed many nonviable HepG2 cells and HUVECs, it was confirmed that their functionality is low. That is, it was confirmed that the NGO-crosslinked scaffold had better biocompatibility than the CTL and GA-crosslinked scaffold, and in particular, NGO-5 is determined to be an optimized condition that satisfies both cell viability and functionality.

### Example 7. Confirmation of Human M2c Macrophage Polarization in NGO Crosslinked Scaffold

Graphene was confirmed to exhibit anti-inflammatory effects by modulating macrophage polarization in models of hepatitis, myocardial infarction and colitis.

Thus, the present inventors performed ex vivo macrophage differentiation to confirm whether NGO in the scaffold could promote the conversion of proinflammatory M1 to anti-inflammatory M2 macrophages. In particular, M2c macrophages are known to mediate matrix remodeling in addition to immune regulation.

Therefore, human peripheral blood-derived mononuclear cell (hPBMC)-derived CD14+ cells were seeded onto NGO-crosslinked scaffolds and differentiated into M1 or M2c macrophages in response to each cytokine stimulus.

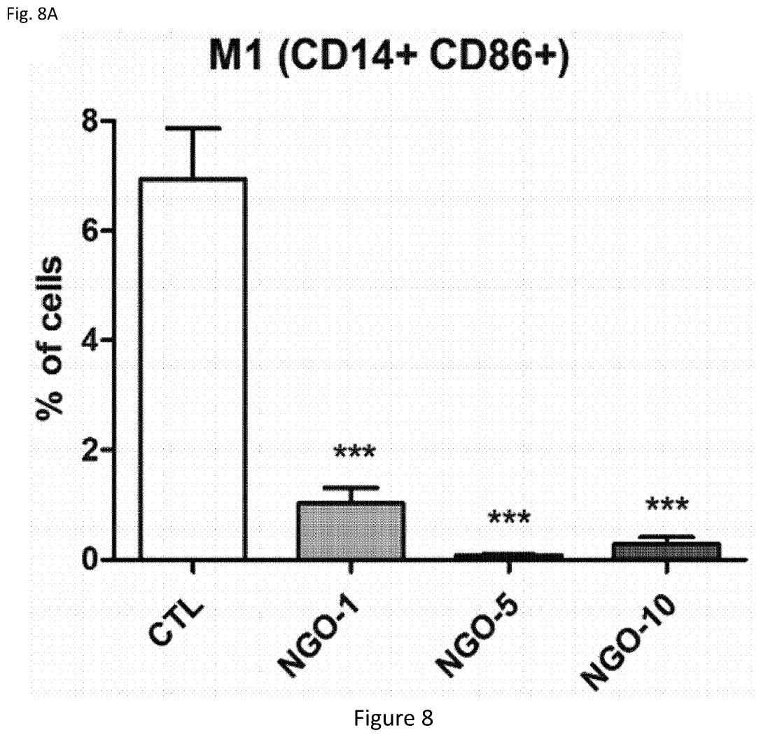

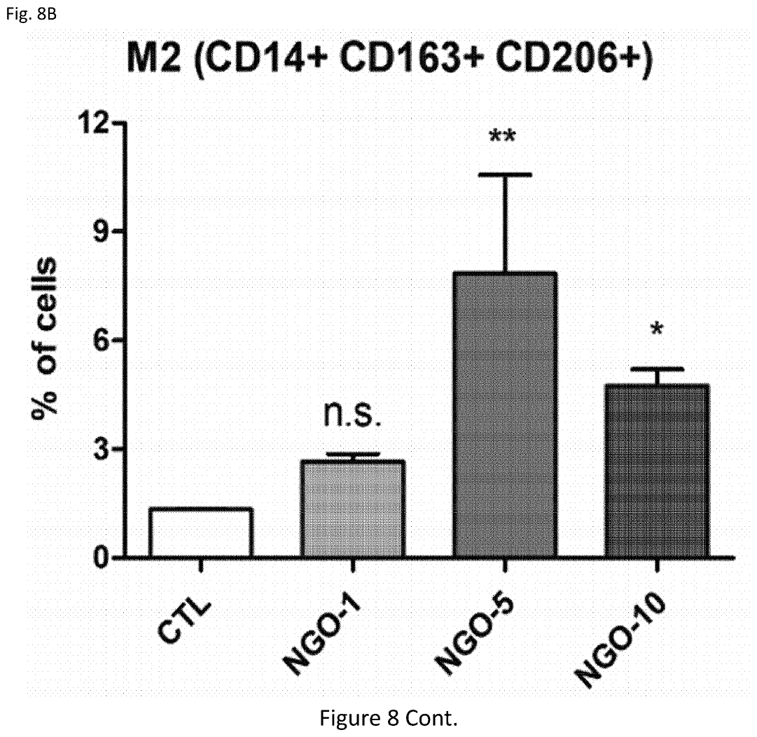

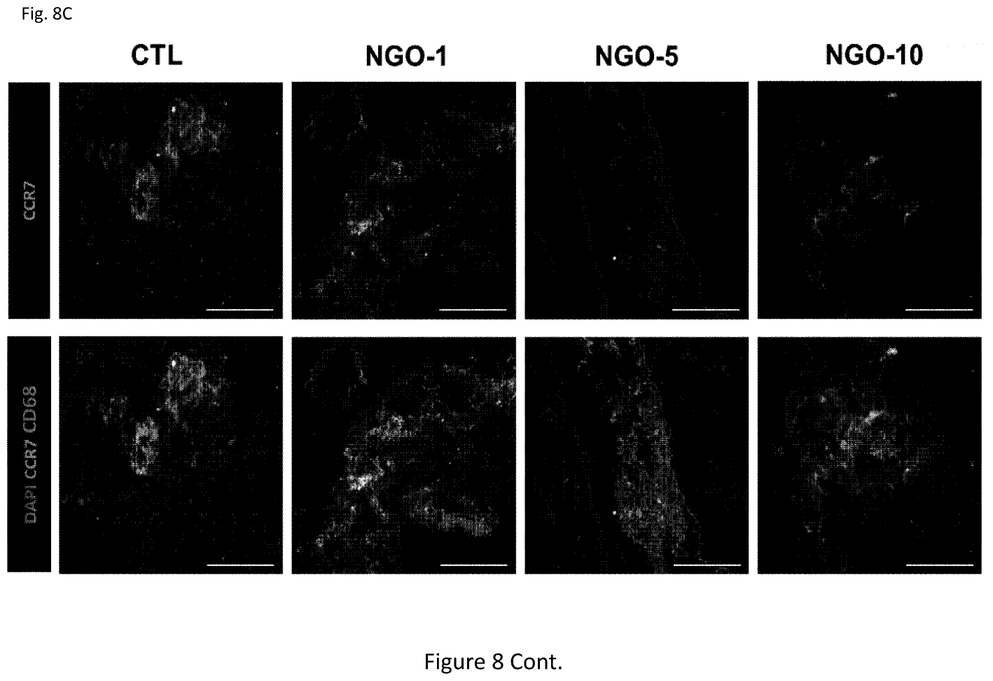

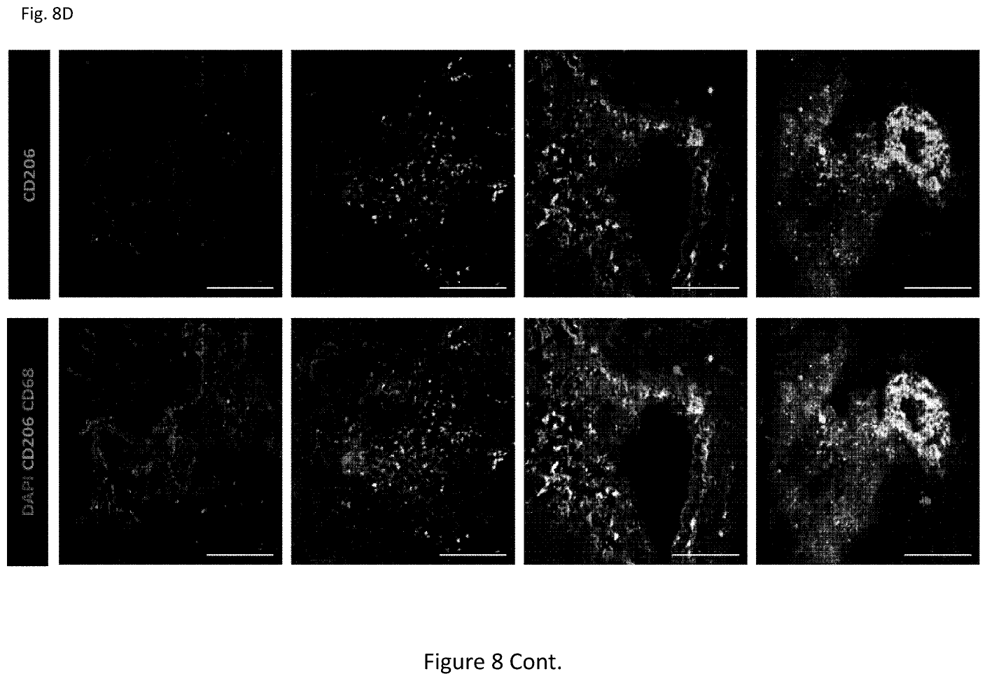

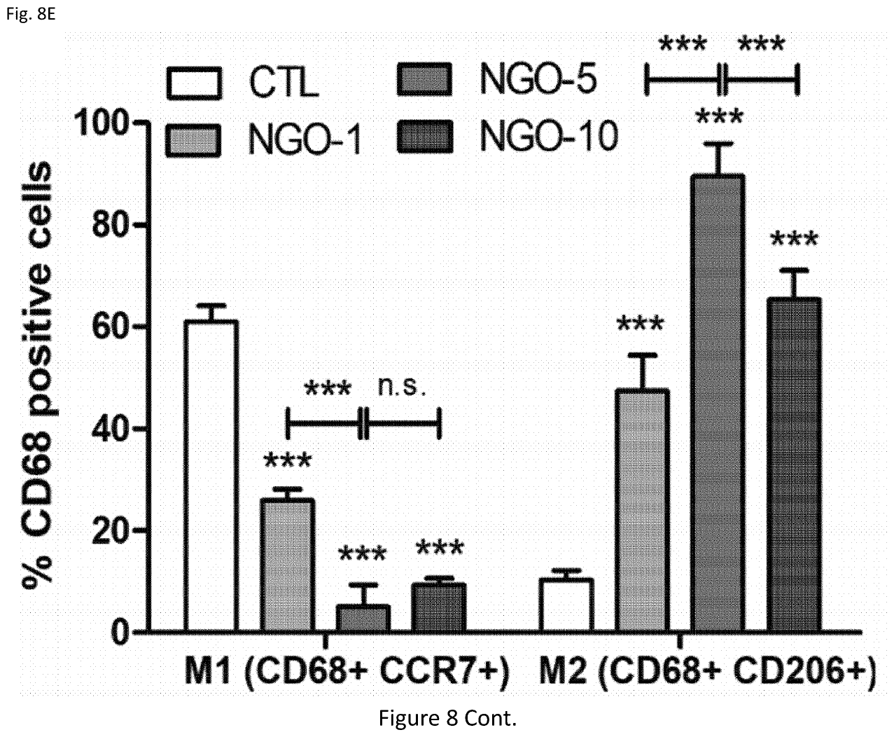

As a result of flow cytometric profiles of human macrophages differentiated in each scaffold showed that NGO crosslinkage suppressed migration to an M1-like phenotype expressing CD86 (see FIG. 8A). In contrast, macrophage polarization to an M2c-like phenotype expressing both CD163 and CD206 was significantly promoted, particularly in the NGO-5 group (see FIG. 8B).

In addition, after immunostaining, the ratio of CCR7⁺ (C—C chemokine receptor 7, M1-specific marker) or CD206⁺ to CD68⁺ (pan-macrophage marker) cells was quantified in each group (see FIGS. 8C to 8E). CD14⁺ cells in the CTL scaffold were predominantly polarized to M1 macrophages, whereas few M1 macrophages were detected in the NGO-5 and NGO-10 groups (see FIG. 8C). Furthermore, crosslinkage by NGO-5 and NGO-10 induced M2 polarization in the scaffold (see FIG. 8D). Although 25.9% of CD68⁺ cells in the NGO-1 group exhibited a phenotype similar to M1, 47.4% of CD68⁺ cells still differentiated into M2 macrophages (see FIG. 8E).

Overall, it is shown that NGO-5 and NGO-10 crosslinked scaffolds may provide an optimized environment for human M2c polarization, showing that the inflammatory response induced by the degradation of the transplanted scaffold and human transplantation may be ameliorated through M2c macrophage infiltration in the presence of NGO.

### Example 8. In Vivo Transplantation of NGO Crosslinked Scaffold

dECM scaffolds are widely known to be degraded gradually upon transplantation. Since various mechanical and chemical stimuli such as enzymes, cytokines and chemokines act in vivo, each crosslinked scaffold was subcutaneously transplanted in order to thoroughly evaluate its susceptibility to the in vivo degradation of the crosslinked scaffold.

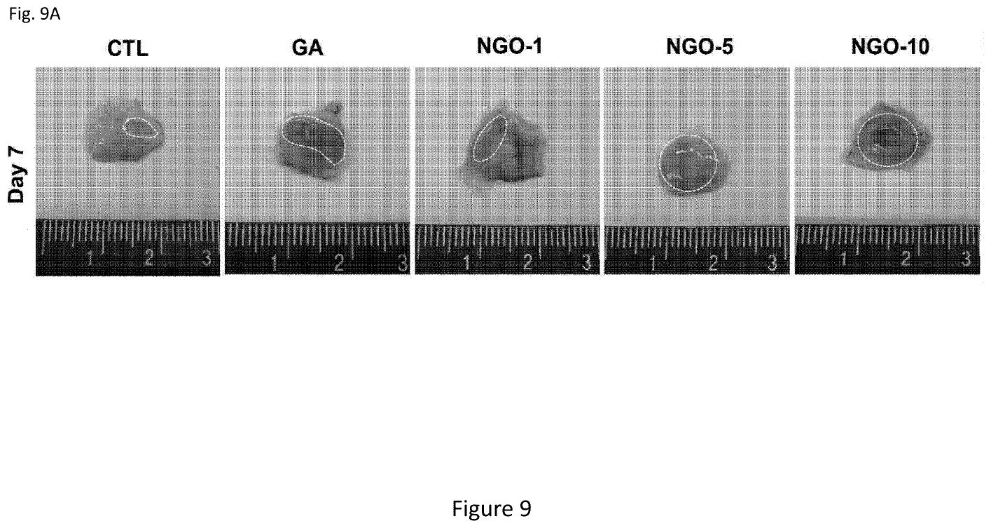

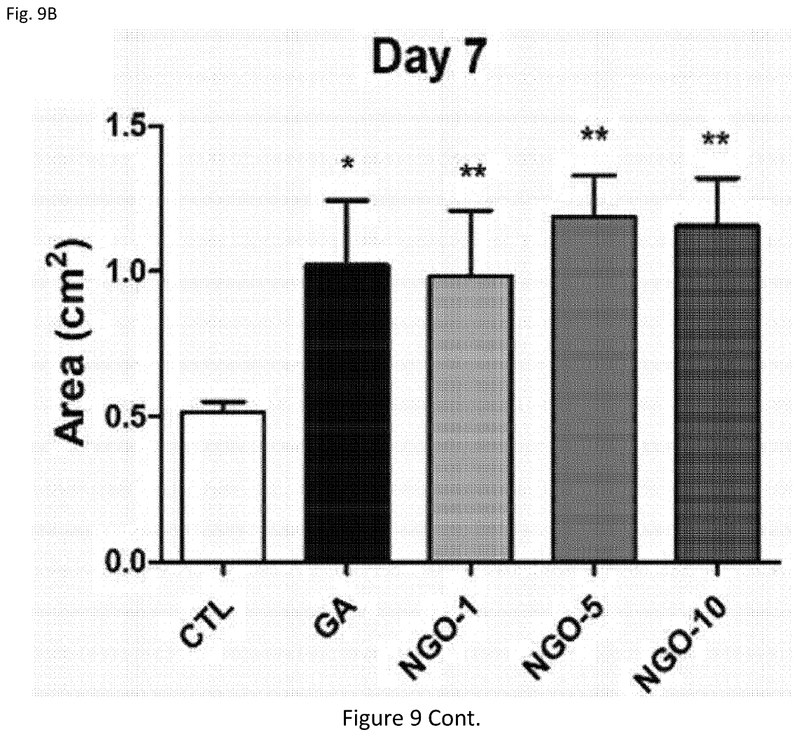

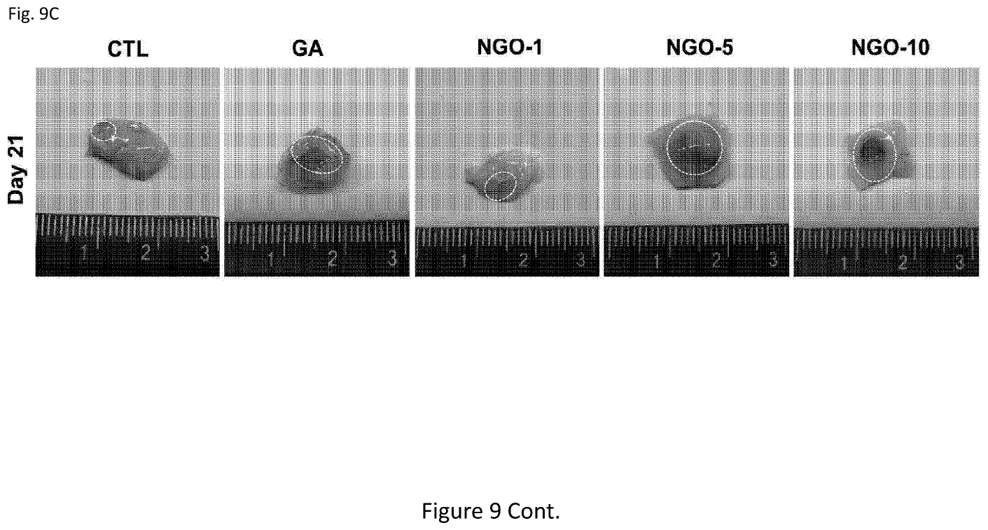

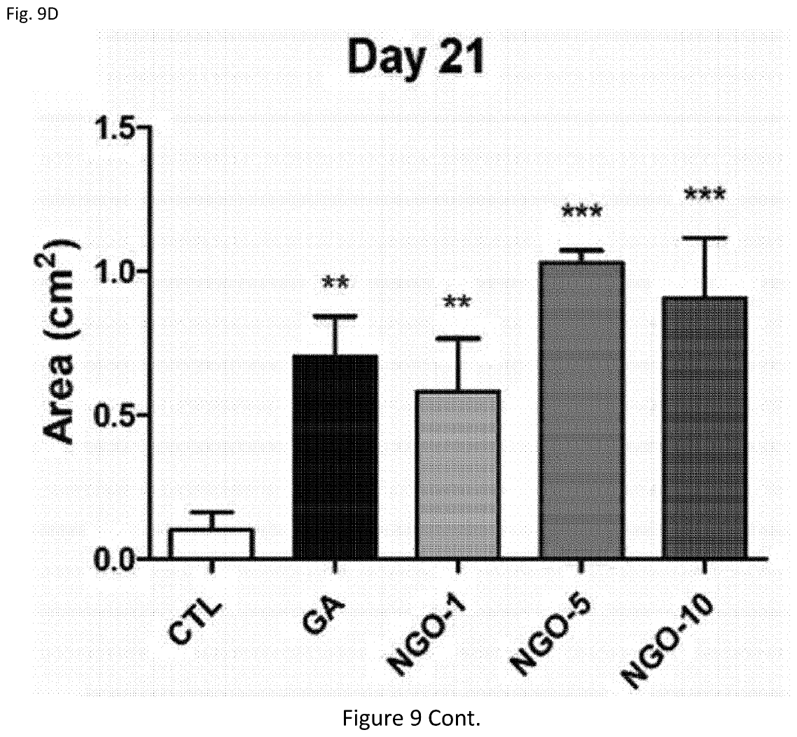

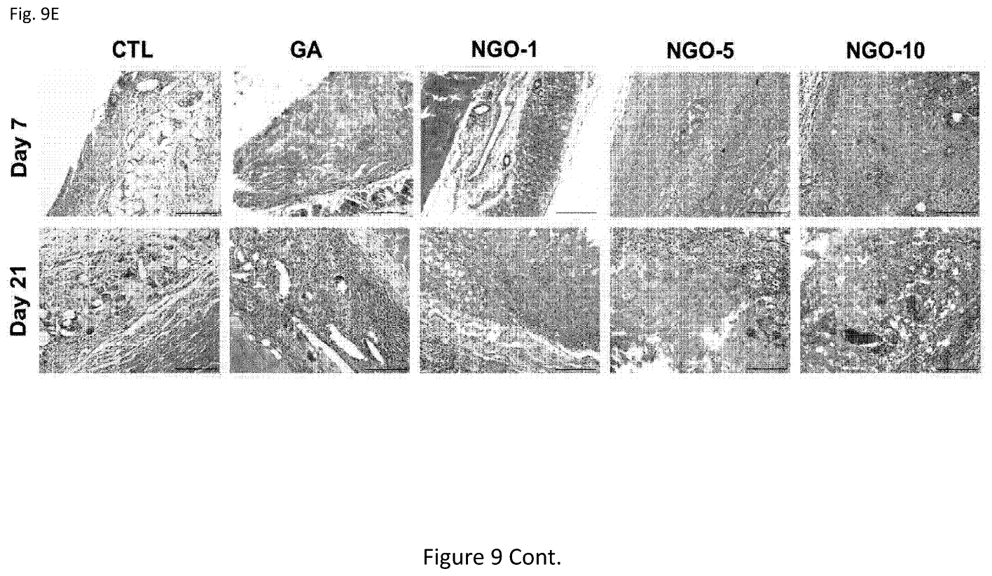

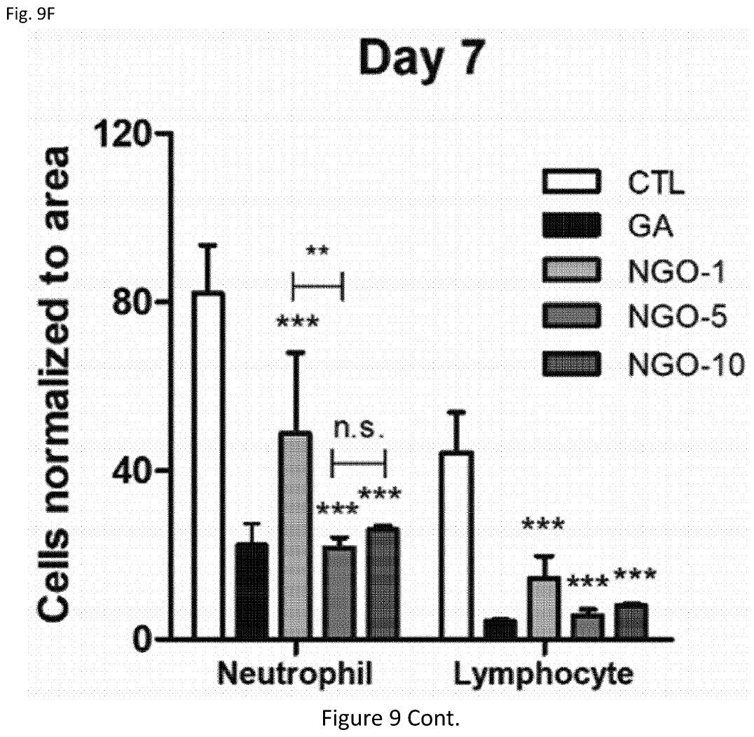

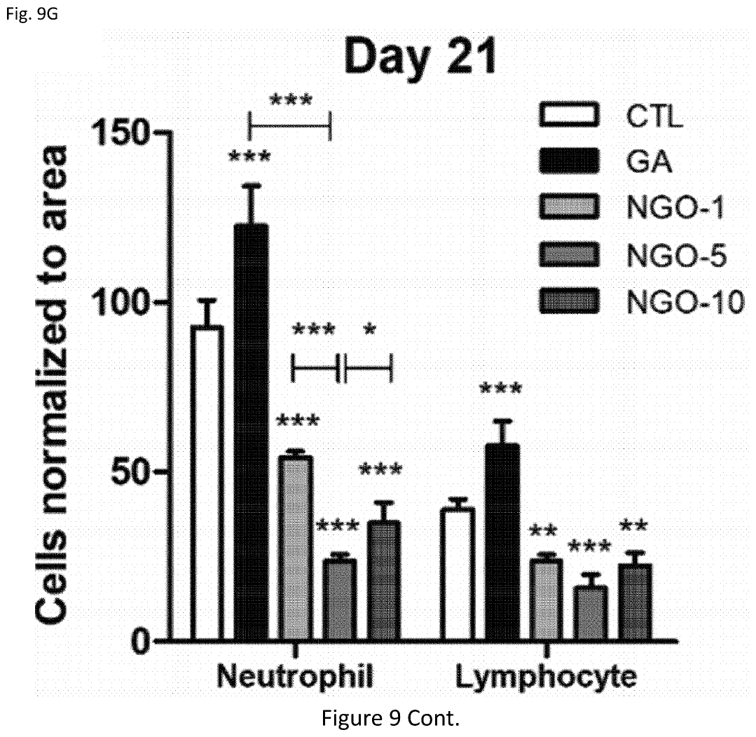

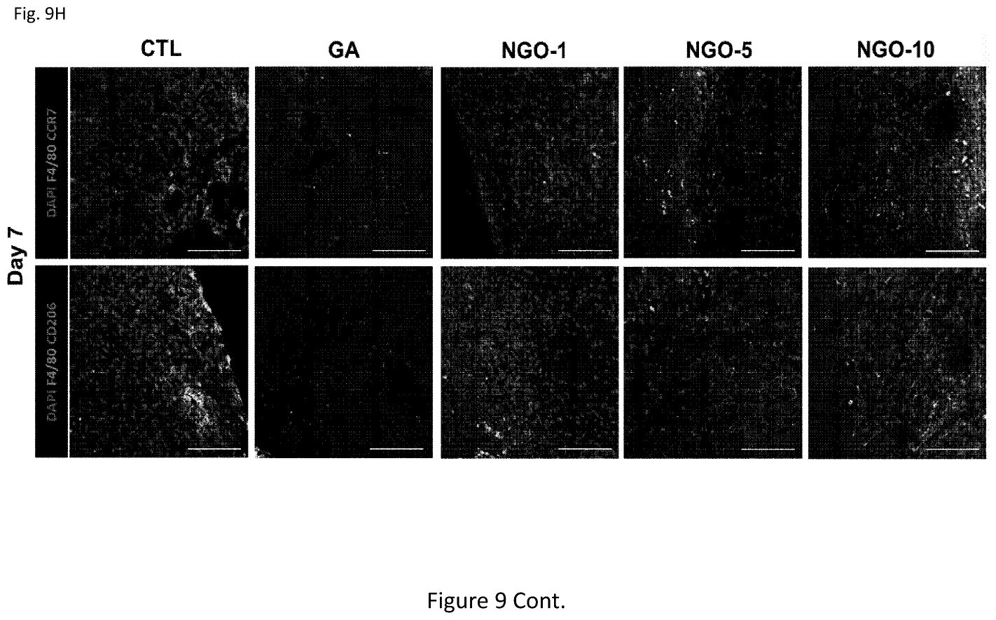

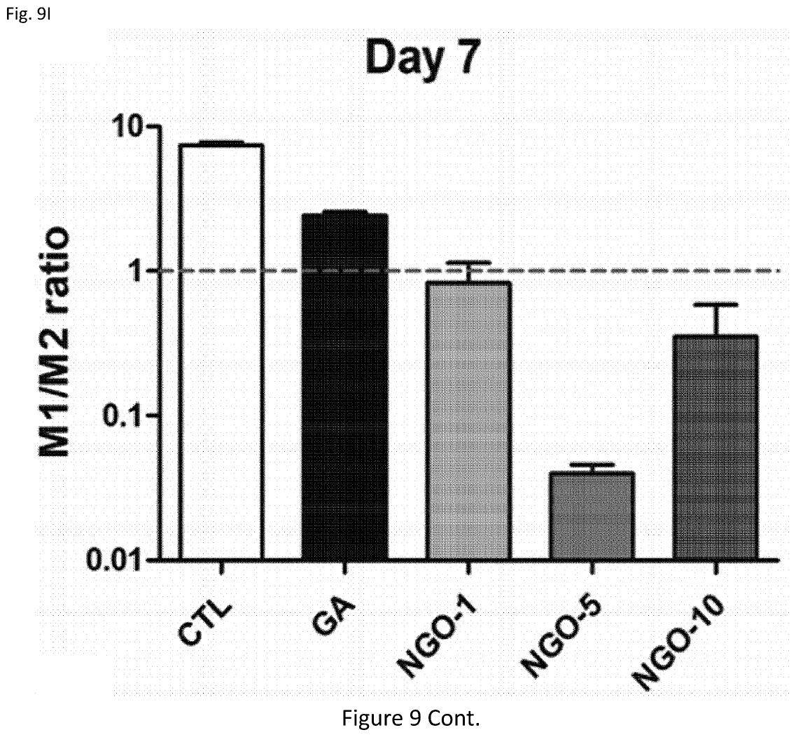

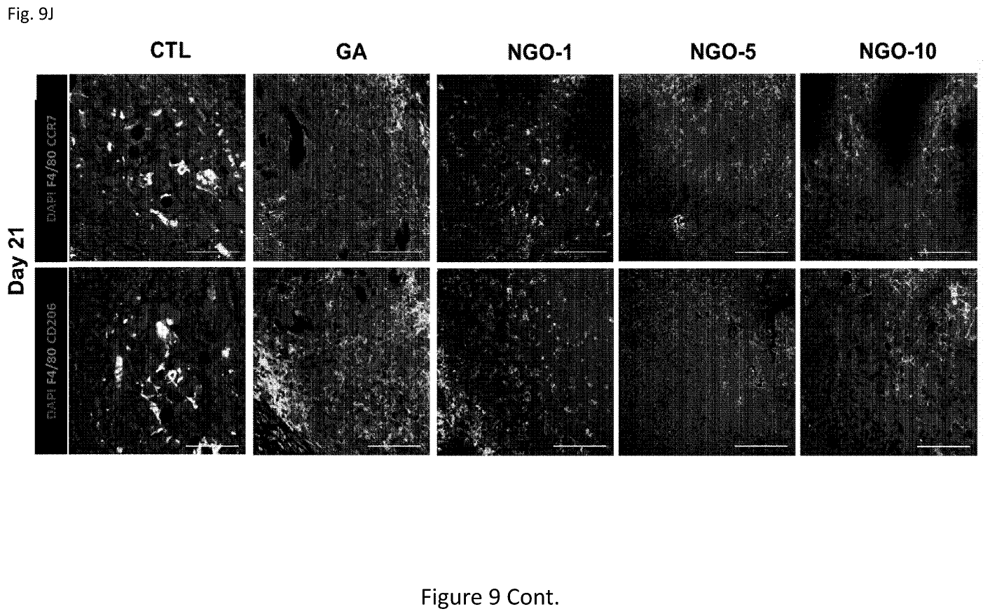

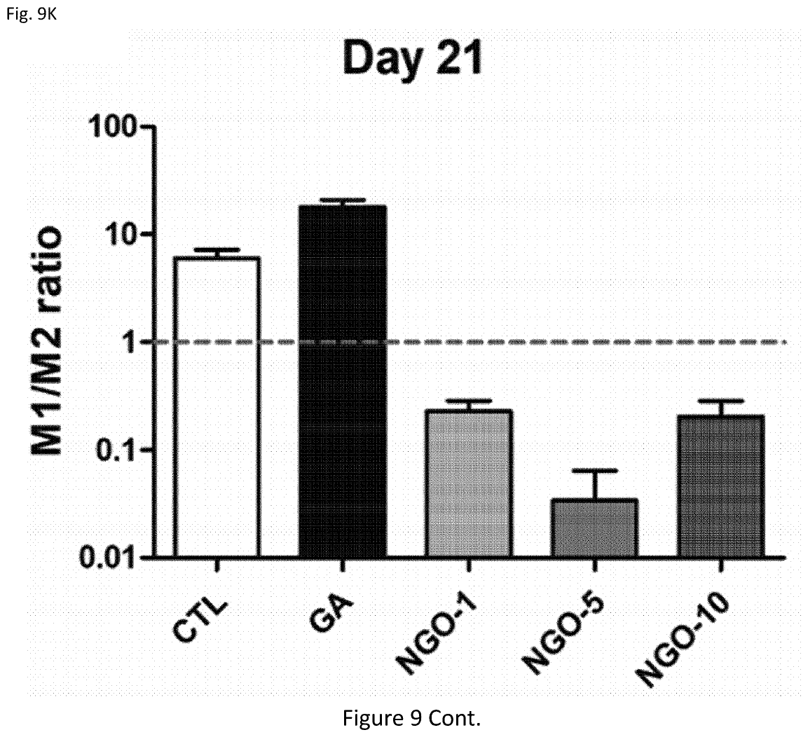

The cross-sectional area of NGO or GA crosslinked scaffolds was at least 2-fold greater than that of the CTL scaffold, as quantified on day 7 after transplantation (see FIGS. 9A and 9B).

In particular, GA, NGO-5 and NGO-10 crosslinked scaffolds remained in considerable amounts up to 21 days after transplantation, whereas the CTL scaffold exhibited a pattern to be rapidly degraded (see FIGS. 9C and 9D).

Further, since dECM scaffolds are known to collect various cell types such as fibroblasts, immune cells and blood cells, invading neutrophils and lymphocytes throughout the scaffold were observed using H&E staining. As a result, it was found that in the NGO crosslinked scaffold, collected inflammatory cells were significantly reduced compared to the CTL scaffold on days 7 and 21 (see FIGS. 9F and 9G).

Next, immunostaining for F4/80 (mouse pan macrophage marker), CCR7 and CD206 was performed to investigate the polarization profile of macrophages that infiltrated the transplanted scaffold. The predominant macrophage type in the scaffold of each group was determined by calculating the ratio of M1 (F4/80 +CCR7⁺ cells) to M2 (F4/80⁺ CD206⁺ cells). Seven days after transplantation, the NGO crosslinked scaffold showed mostly M2 macrophages in spite of partial M1 expression in the NGO-1 group (see FIGS. 9H and 9I). M1 expression was remarkably increased in the CTL and GA groups on day 21, whereas M2 expression still remained predominant in the NGO crosslinked scaffold (see FIGS. 9J and 9K). That is, according to the ex vivo macrophage polarization results, it was proved that the NGO-5 group exhibited the strongest effect on in vivo M2 polarization which mediates anti-inflammatory responses.

### Example 9. Remodeling of Transplanted Scaffold Mediated by M2c Macrophages

An underlying mechanism in which NGO protects a scaffold after transplantation from degradation was explored. M2 macrophages are known to regulate structural tissue remodeling by releasing both MMP and TIMP (tissue inhibitors of metalloproteinase). In particular, TIMP-1 is expressed primarily by M2c macrophages, although it is also partially expressed by M1 or M2a macrophages. Therefore, the expression levels of MMP-9 and TIMP-1 were investigated at the transplantation site.

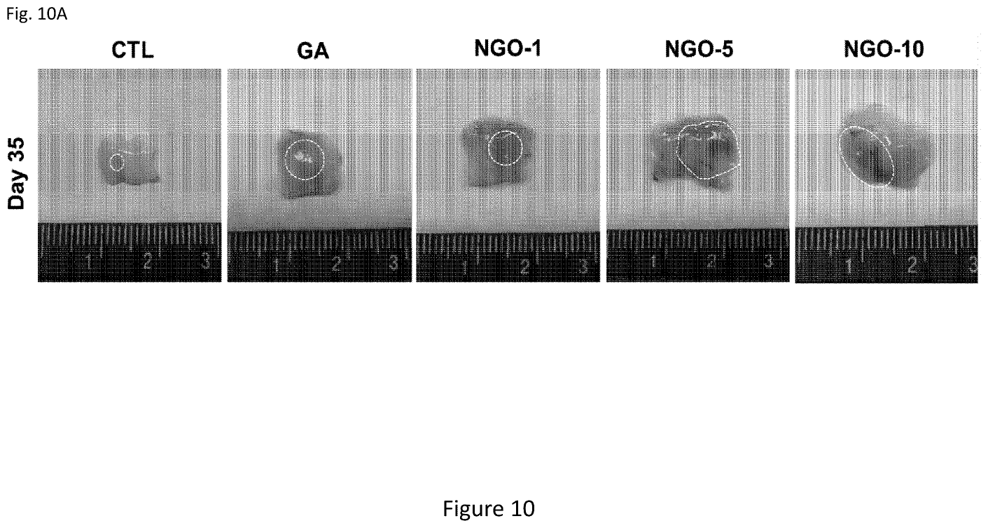

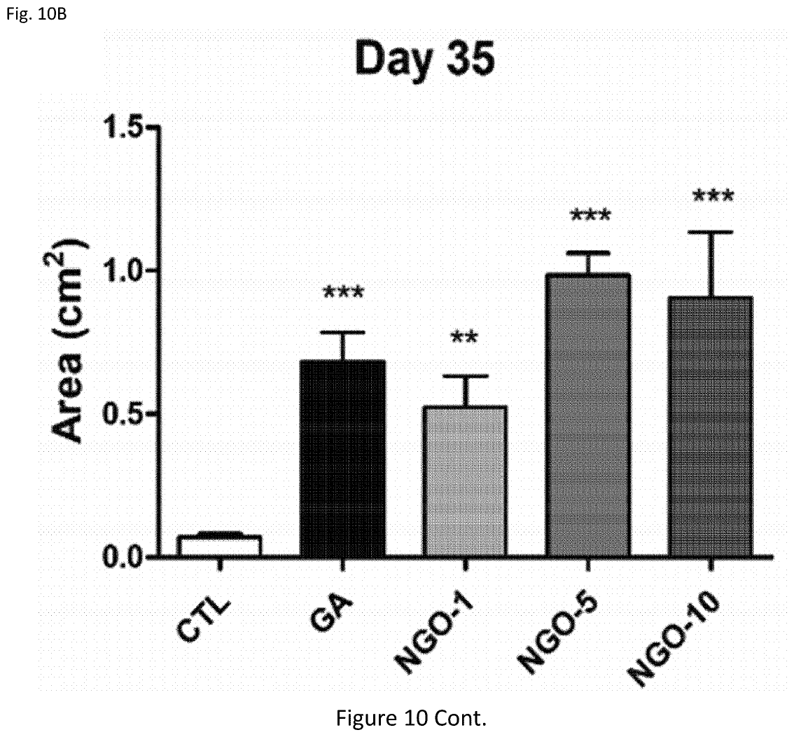

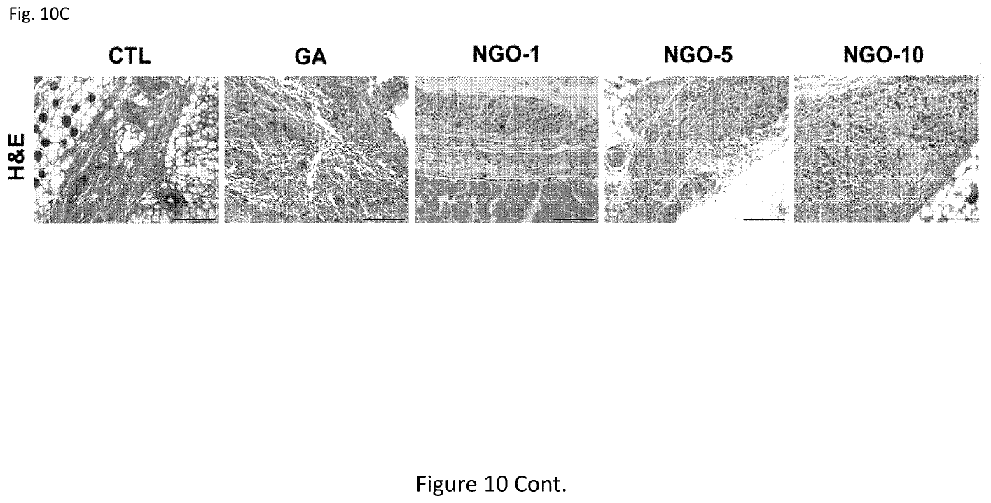

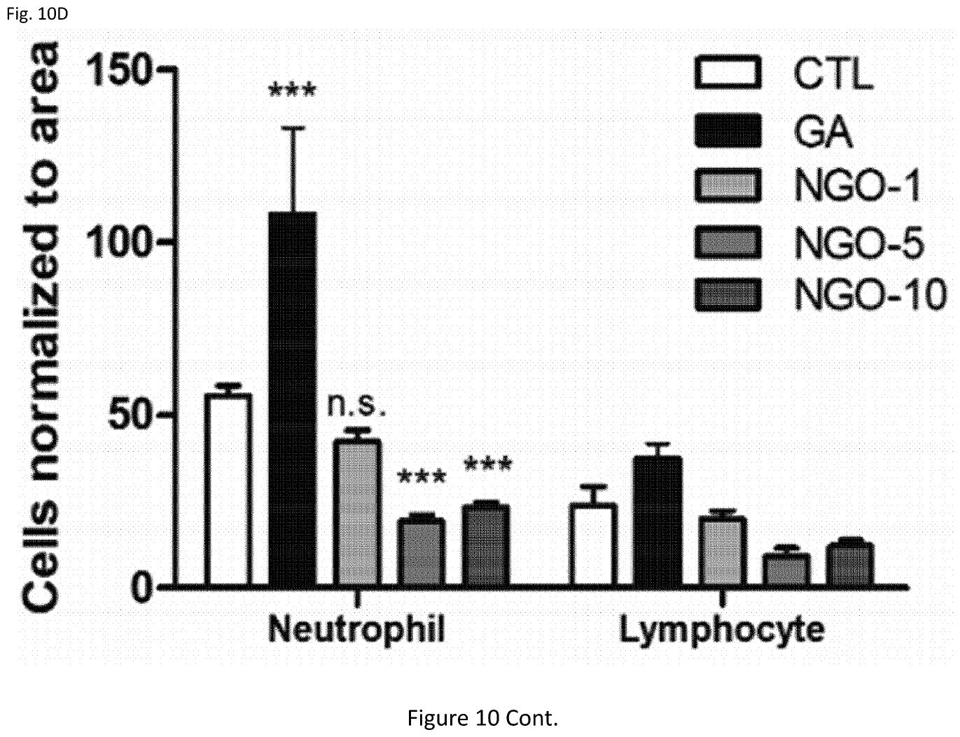

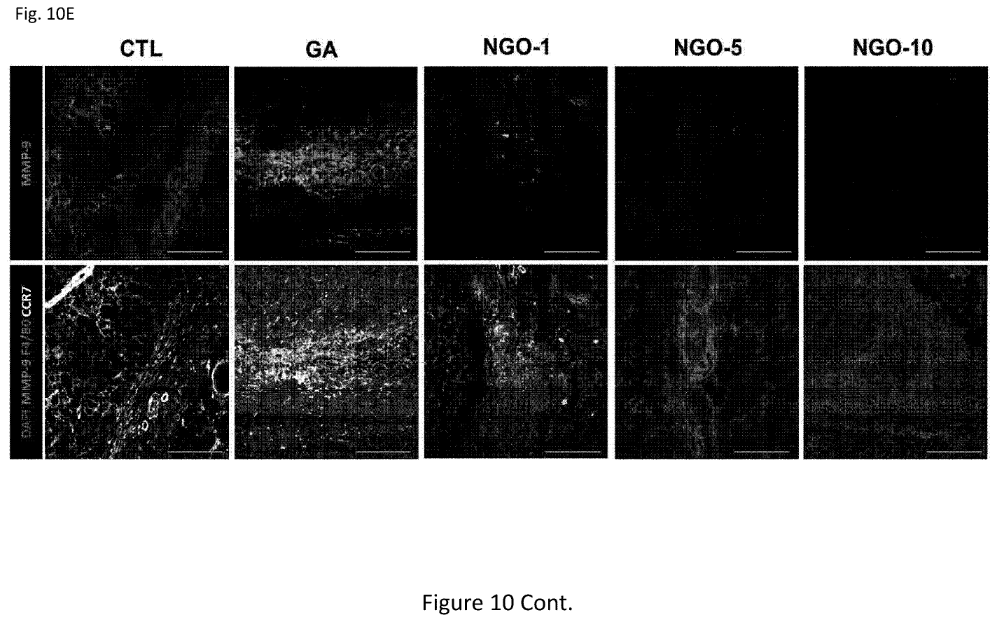

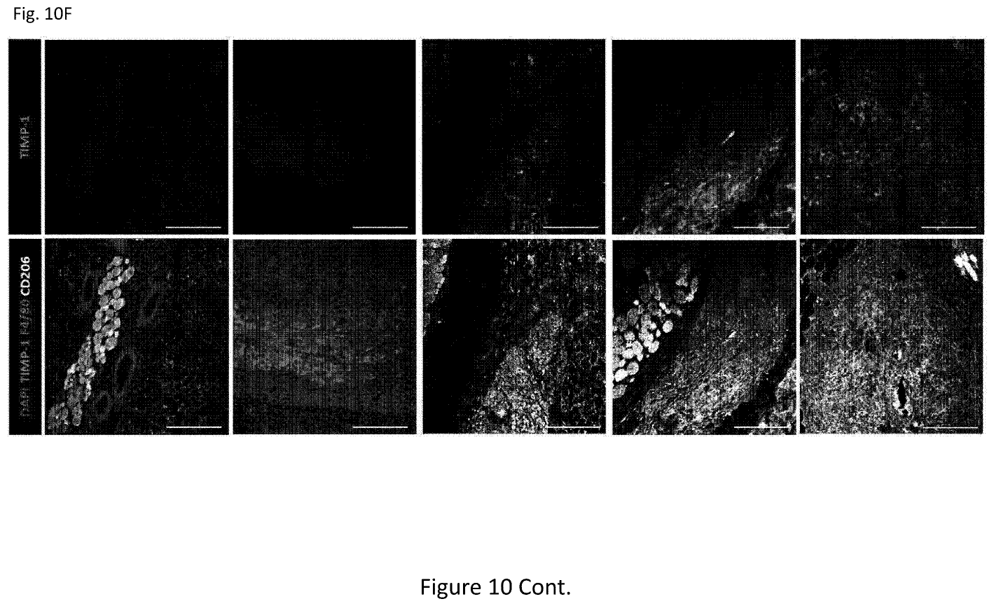

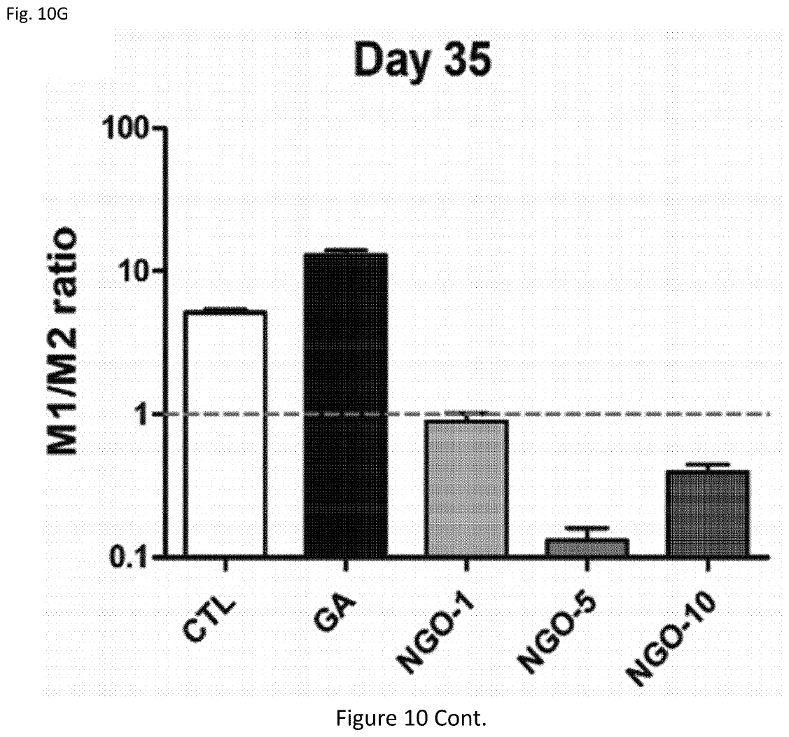

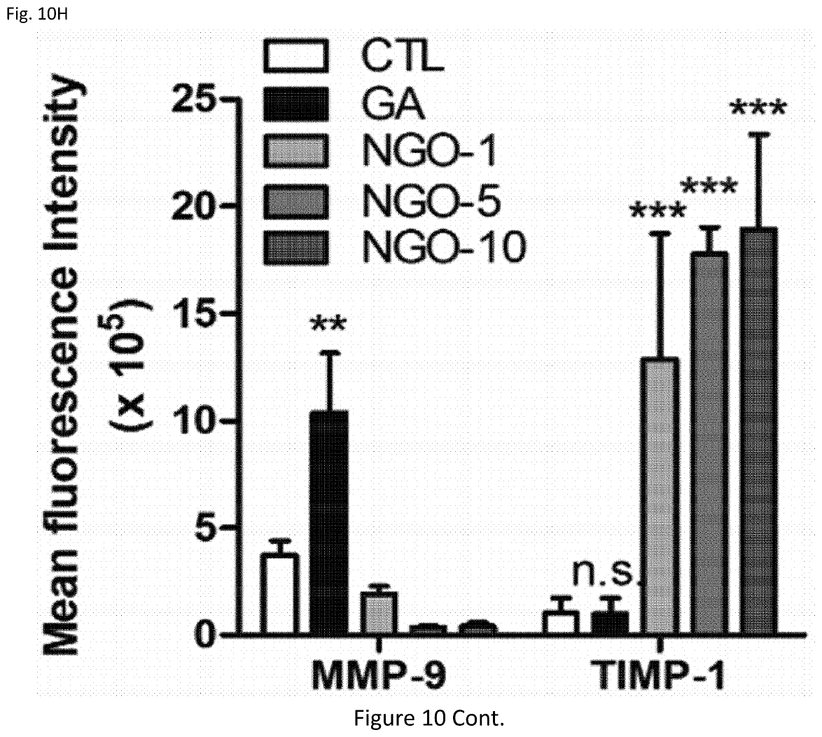

As a result, it was found that in the NGO-5 and NGO-10 crosslinked scaffolds, the infiltration of CD206⁺ M2c macrophages expressing TIMP-1 was remarkable, whereas MMP-9 expression was relatively deficient (see FIGS. 10A to 10D). In addition, TIMP-2 was highly expressed in the NGO crosslinked scaffold, whereas MMP-2 levels were very low (see FIG. 10H). Considering that some M2 macrophages express MMP, the above results imply that NGO interacted with the released MMP and suppressed their activity.

Overall, it was confirmed that NGO suppresses in vivo degradation by promoting macrophage polarization to M2, which is a source of TIMP not only directly suppressing MMP, but also suppressing MMP activation.

### Example 10. Long-Term Maintenance of In Vivo NGO Crosslinked Scaffold

To confirm the effect of NGO on scaffold degradation resistance, transplants were maintained for a long period of time.

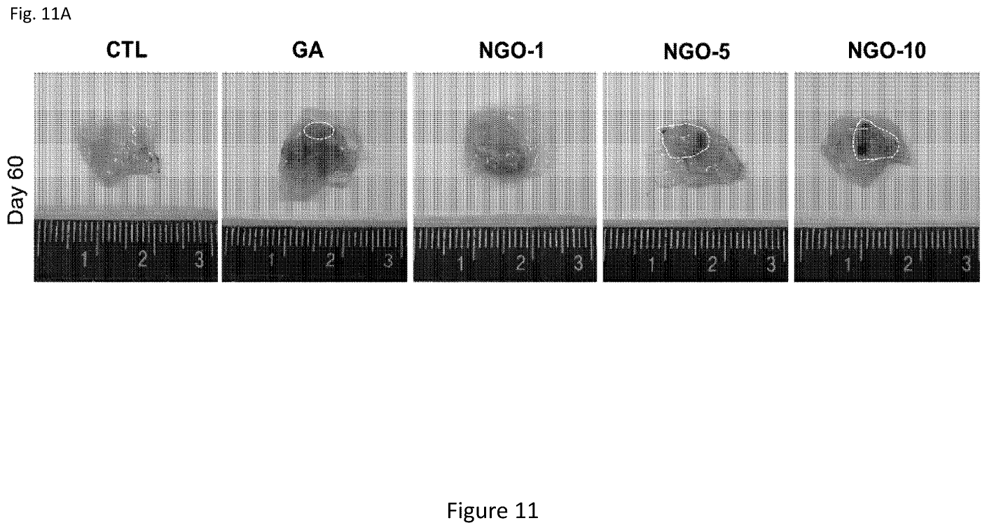

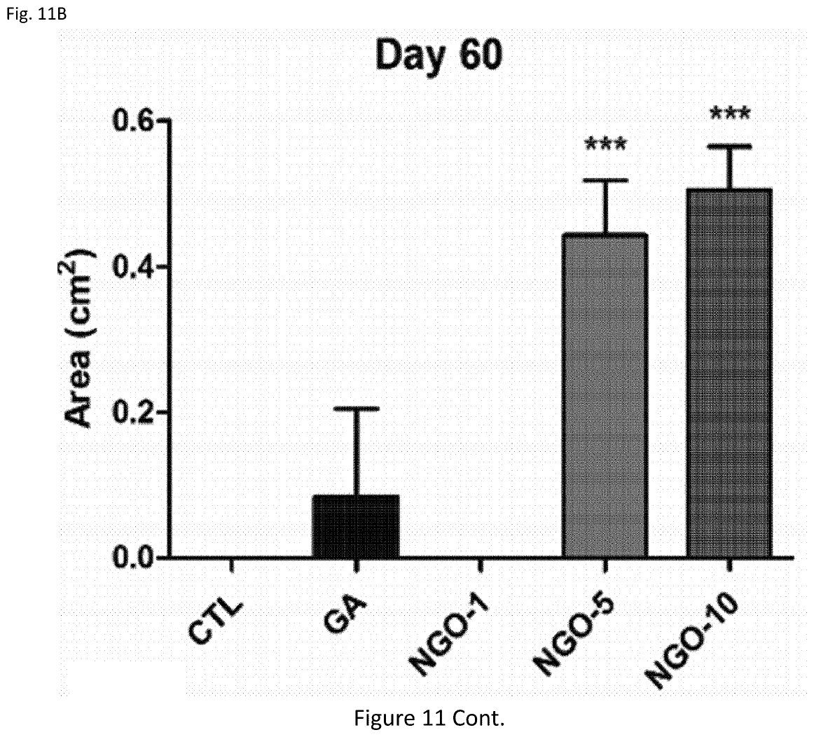

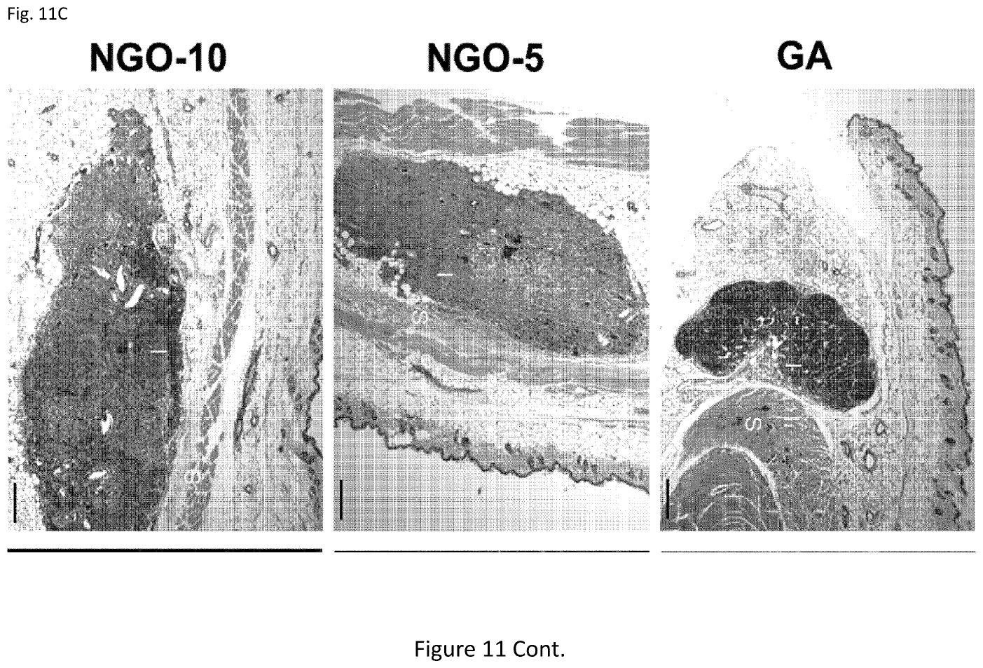

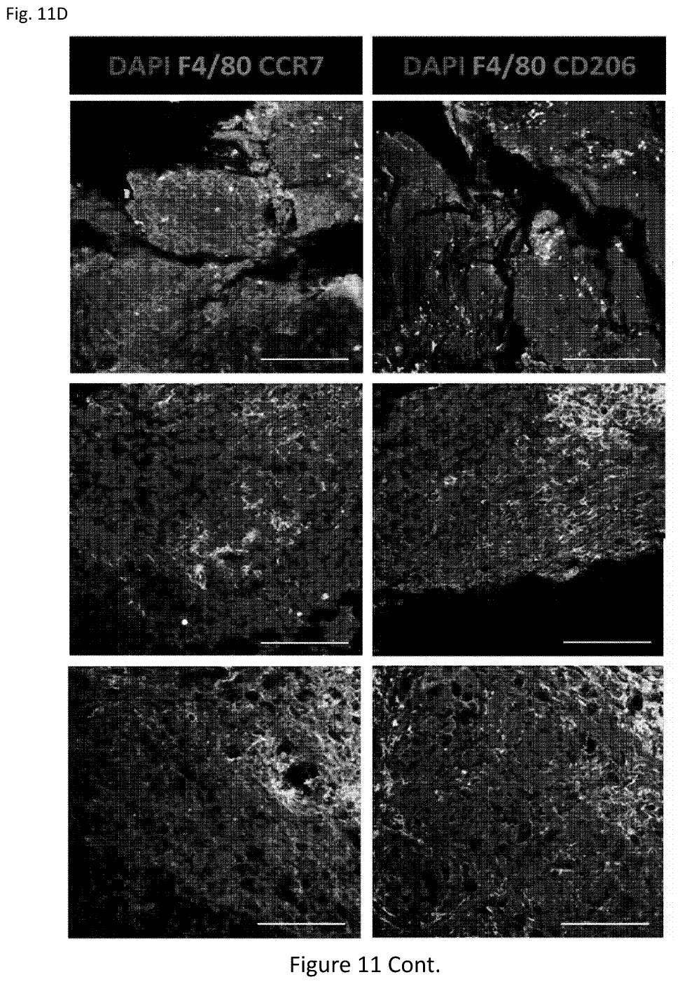

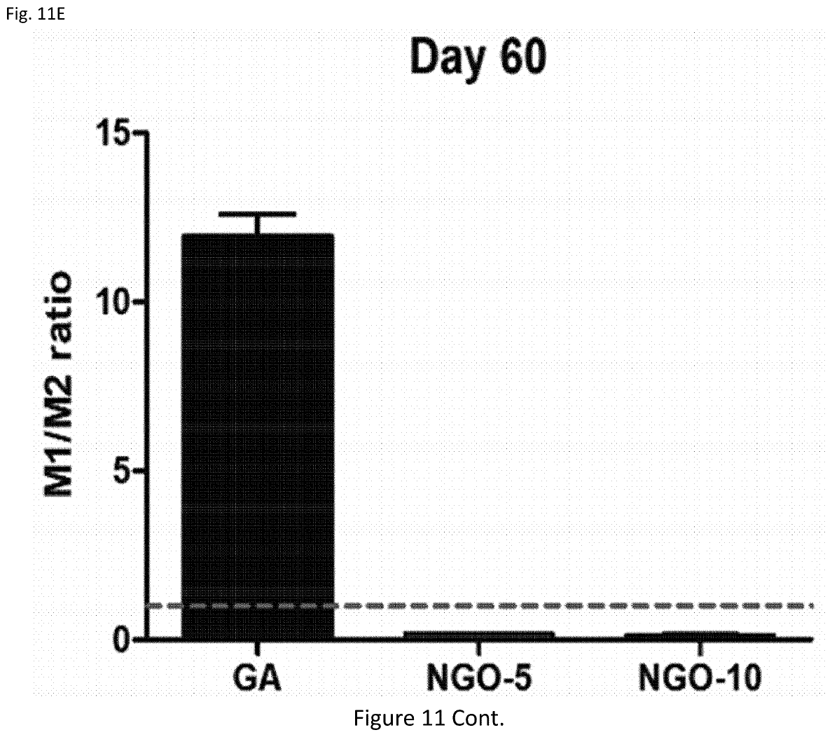

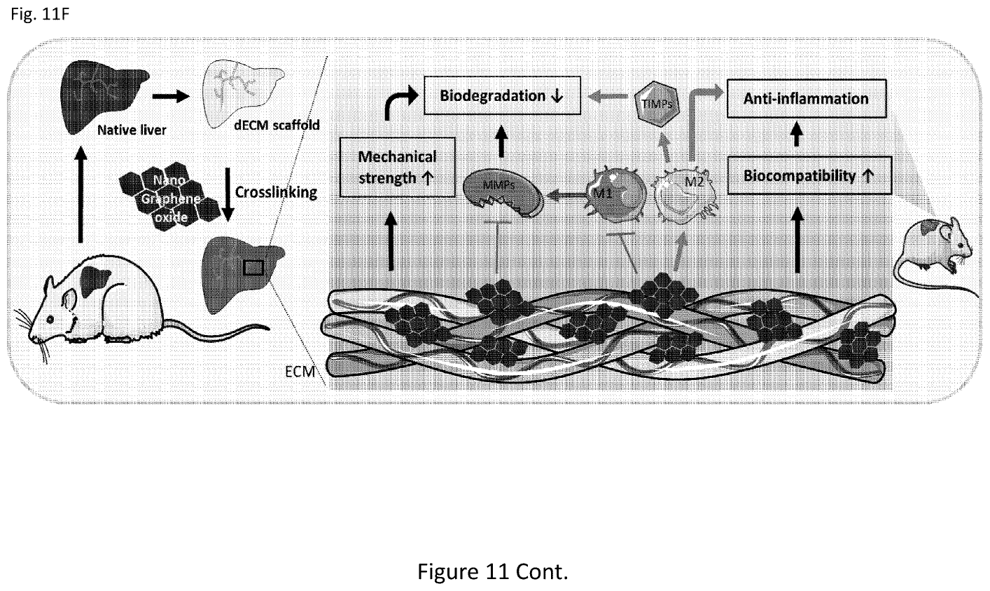

As a result, NGO-5 and NGO-10 crosslinked scaffolds were very stable even 60 days after transplantation, whereas CTL and NGO-1 crosslinked scaffolds were completely degraded (see FIGS. 11A and 11B). Furthermore, the NGO-5 group showed a relatively low level of inflammatory response up to 60 days compared to the other groups (see FIG. 11C). Further, the NGO crosslinked scaffold showed an effect of suppressing scaffold degradation mainly through a favorable mononuclear cell response that migrated to M2 macrophages (see FIGS. 11D and 11E). The GA-crosslinked scaffold remained partially up to 60 days, but were found to exacerbate inflammation systemically compared to the NGO crosslinked scaffold (see FIGS. 11A to 11C). In addition, the GA crosslinked scaffold showed that M1 macrophages were strongly infiltrated, and thus severe inflammatory response and MMP-mediated degradation continuously proceeds (see FIGS. 11D and 11E).

Overall, it was confirmed that NGO crosslinkage has a significant effect on maintaining the structural integrity of the scaffold in vivo and attenuating inflammation after transplantation. Furthermore, since NGO-1 was insufficiently effective in suppressing degradation, it was confirmed that NGO-5 was the optimized condition for both crosslinkage and immunomodulation.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

The present inventors have revealed that when nano graphene oxide is used as a crosslinking agent for decellularized scaffolds, not only the physical properties of decellularized scaffolds are strengthened, but also the inflammatory response induced within the scaffold or systemically after transplantation is alleviated. In addition, a new mechanism that nano graphene oxide not only promotes polarization to M2c macrophages to promote anti-inflammatory responses, but also suppresses MMP secretion to suppress the biodegradation of the scaffold was confirmed. This is useful in that nano graphene oxide not only increases the engraftment rate of transplants during the production of an artificial organ, but also allows the decellularized scaffold to be maintained in vivo for a long period of time.

Therefore, it is possible to produce an artificial organ in which various cells are applied to a transplantable scaffold prepared using nano graphene oxide, which is expected to contribute to the improvement in the quality of life and the extension of the lifespan of patients at home and abroad as an alternative treatment for organ transplantation. Further, nano graphene oxide is expected to be widely used in terms of tissue engineering, such as application to not only a decellularized organ scaffold, but also a synthetic biomaterial, and thus has industrial applicability.

1-25. (canceled)

26. A method for producing an artificial organ, the method comprising performing treatment with a composition comprising a graphene nanostructure as an active ingredient.

27. The method of claim 26, further comprising the following steps:
a) providing an organ from a tissue source;
b) decellularizing the provided organ; and
c) treating the decellularized organ with a graphene nanostructure.

28. The method of claim 27, further comprising one or more steps selected from the group consisting of the following steps:
d-1) recellularizing a decellularized organ into vascular endothelial cells;
d-2) recellularizing a decellularized organ into parenchymal cells; and
d-3) recellularizing a decellularized organ into non-parenchymal cells.

29. The method of claim 26, wherein the graphene nanostructure is a nano-sized graphene oxide or graphene quantum dot.

30. The method of claim 29, wherein the nano-sized graphene oxide has a thickness of 20 nm or less; or an average diameter of 15 to 50 nm.

31. The method of claim 26, wherein the graphene nanostructure is crosslinked to a decellularized scaffold.

32. The method of claim 26, wherein the graphene nanostructure strengthens or improves the physical properties of a decellularized scaffold.

33. The method of claim 32, wherein the physical properties are one or more selected from the group consisting of the following properties:
i) elasticity of the decellularized scaffold;
ii) weight loss due to degrading enzymes of the decellularized scaffold; and
iii) tensile strength of the decellularized scaffold.

34. The method of claim 26, wherein the graphene nanostructure suppresses the in vivo degradation of a decellularized scaffold by directly suppressing a matrix metalloproteinase (MMP).

35. The method of claim 26, wherein the graphene nanostructure ameliorates or suppresses the induction of inflammatory response of a decellularized scaffold.

36. The method of claim 26, wherein the graphene nanostructure alleviates post-transplantation inflammatory responses by promoting polarization to M2 macrophages.

37. The method of claim 26, wherein the composition further comprises parenchymal cells.

38. The method of claim 37, wherein the parenchymal cells are stem cell-derived parenchymal cells.

39. The method of claim 38, wherein the stem cells are one or more selected from the group consisting of induced pluripotent stem cells (iPSCs), embryonic stem cells, marrow-derived stem cells, adipose tissue-derived stem cells and placenta-derived stem cells.

40. The method of claim 26, wherein the organ is the liver.
